(19) United States
(12) Patent Application Publication
(10) Pub. No.: US 2017/0360861 A1
(43) Pub. Date: Dec. 21, 2017

(54) METHODS FOR EXTRACTING TARGET COMPOUNDS FROM CANNABIS

(71) Applicant: GREEN SKY LABS, INC., Victoria (CA)

(72) Inventors: JAMES DOUGLAS HUMPHREYS, Victoria (CA); Jay Paul VAN DER VLUGT, Victoria (CA)

(73) Assignee: GREEN SKY LABS, INC., Victoria, BC (CA)

(21) Appl. No.: 15/535,385

(22) PCT Filed: Dec. 14, 2015
(86) PCT No.: PCT/IB2015/002491

§ 371 Date: Jun. 12, 2017

Related U.S. Application Data
(60) Provisional application No. 62/091,452, filed on Dec. 12, 2014.

(51) Int. Cl. A61K 36/185; A61K 31/352; B01D 11/02

(52) U.S. Cl. CPC A61K 36/185; B01D 11/0288; A61K 31/352; A61K 2236/333; A61K 2236/53

(57) ABSTRACT

Botanical materials are treated by processes utilizing a solvent system that includes the use of acetone solvent with or without a CO₂ co-solvent wherein the solvent system is allowed to process the botanical material under certain conditions to obtain extracts (including Whole Plant Extracts) of the botanical materials that are substantially free of pigments, waxes, fats, lipids, and the like.

### CROSS-REFERENCE TO RELATED APPLICATIONS

This patent application claims the benefit of US Provisional Patent Application No. 62/091,452, filed Dec. 12, 2014, the entire contents of which is incorporated herein by reference.

### BACKGROUND

Methods and/or apparatus/instrumentation are disclosed herein for extracting and isolating target compounds, such as terpenes and terpenoids (e.g., cannabinoids such as tetrahydrocannabinol (THC), cannabidiol (CBD), plant essential oils, etc.), alkaloids (e.g., nicotine), esters, amines, aromatics, alcohols, aldehydes, ketones, lactones, thiols, and other volatile species found in, or derived from, botanical source materials, such as, for example compounds derived from hops, hemp, cannabis, tobacco, etc. (As used herein, “extract” refers to a substance obtained by extracting a raw material, using a solvent system.)

For example, cannabinoids are increasingly being used for pharmaceutical and nutraceutical applications. Cannabinoids are compounds derived from an annual plant in the Cannabaceae family. There have been identified about 400 cannabinoids. Although the relative percentage of cannabinoids in Cannabis plants varies greatly with genetic and environmental factors, major constituents typically include the tetrahydrocannabinols (collectively referred to as THC), cannabidiol (CBD) and cannabinol (CBN) along with minor constituents such as cannabichromene (CBC). Cannabis sativa has a higher level of THC compared to CBD, while Cannabis indica has a higher level of CBD compared to THC. It has been observed that Cannabis strains with relatively high CBD:THC ratios are less likely to induce anxiety than vice versa. This may be due to CBD's antagonistic effects at the cannabinoid receptors, compared to THC's partial agonist effect. CBD is also a 5-HT1A receptor (serotonin) agonist, which may also contribute to an anxiolytic-content effect. This likely means the high concentrations of CBD found in Cannabis indica mitigate the anxiogenic effect of THC significantly. The effects of sativa are well known for its cerebral high, while indica is well known for its sedative effects, which some prefer for night time use. Both types are used for medicinal purposes. For instance, THC and CBD are used for the treatment of a wide range of medical conditions, including glaucoma, AIDS wasting, neuropathic pain, treatment of spasticity associated with multiple sclerosis, fibromyalgia and chemotherapy-induced nausea. Additionally, THC has been reported to exhibit a therapeutic effect in the treatment of allergies, inflammation, infection, epilepsy, depression, migraine, bipolar disorders, anxiety disorder, and drug dependency and withdrawal syndromes. THC is particularly effective as an anti-emetic drug and is administered to curb emesis, a common side effect accompanying the use of opioid analgesics and anesthetics, highly active anti-retroviral therapy and cancer chemotherapy.

Cannabinoid compounds used in such applications are almost exclusively obtained from natural sources, for example, from plant tissue. Cannabinoid compounds are obtained from, for example, the trichomes of the sativa plant using various methods, including solvent extraction methodologies. Some draw backs associated with such methods include poor or inconsistent yields, high costs associated with growing and maintenance of the cannabis plant and costs associated with extraction and purification of extract and toxicity of such extraction solvents. Government regulations and security for cannabis plants are also an important consideration that adds to the overhead cost of producing extracts containing cannabinoid compounds.

Further, consumers of smoking or vaporizing articles are sensitive to a variety of characteristics that contribute to a pleasurable smoking or vaporizing experience, including among others the aroma of the smoking or vaporizing article itself, the aroma and flavour (“essences”) of the smoke or vapor generated by the smoking or vaporizing article upon ignition thereof, and the “mouthfeel” created by the smoke or vapor generated by the smoking or vaporizing article that has been inhaled. The term “mouthfeel” refers to the impact, body and other sensations (e.g., harshness, peppery, powdery, etc.) of the smoke or vapor produced upon ignition of the smoking article and inhalation of the smoke or vapor produced therefrom in the user's mouth. For example, the presence of undesirable plant constituents in a botanical extract of cannabis, tobacco, etc., such as chlorophyll, waxes, etc., is believed to impart a harsh or otherwise distasteful mouthfeel. As such, it will be advantageous for a botanical extraction method that is capable of isolating only desirable constituents or essences that impart a preferred mouthfeel or flavor without the above-mentioned undesirable constituents.

From a technical standpoint, conventional botanical extraction methods using non-aqueous solvents and the like are too crude or too complex, inefficient, time consuming, and/or expensive. Conventional methods of extraction that have been used to separate the above and other constituents of botanical materials, and to produce enriched extracts of same, include maceration, decoction, and extraction with aqueous and non-aqueous solvents, distillation and sublimation.

While there is a wide variety of extraction technologies to be applied to botanical materials, such extraction methodologies do not retain as many extracted target molecules once solvent is removed. In particular, no conventional extraction technology provides an optimum system where desirable target molecules are efficiently separated from a botanical material and dissolved into a solvent without concurrently extracting a high yield of undesirable wax and pigment molecules that decrease the purity and quality of the extract solution. Furthermore extraction solvents used in current methodologies are not effectively removed from the extracted materials without significant simultaneous loss of target molecules.

The “traditional” approach was to produce a decoction (by boiling the plant material in water) or to produce a solvent extract, e.g. an ethanolic extract (by, for example, reflux) and use either of those as a medicine.

Methods of extraction which have been used to separate constituents of plant medicines and to produce enriched extracts include maceration, decoction, and extraction with aqueous and non-aqueous solvents, distillation and sublimation. For example, maceration (softening by soaking) and decoction (concentrating by heating or boiling) methods rely on a short diffusion path. Constituents such as lecithins, flavonoids, glycosides and sugars are released and, in some cases, may act to solubilize other constituents which, in the pure state, are really soluble in the solvent. As such, a disadvantage of maceration and decoction with water or low concentrations of ethanol is that a large quantity of inert material that does not have medicinal value is extracted. Inert material may consist of plant cell constituents including, but not limited to, fats, waxes, carbohydrates, proteins and sugars, which may contribute to microbiological spoilage if the product is not administered promptly. If dried, the extracts so produced by these methods tend to be hygroscopic and difficult to formulate. The inner material may also affect the way in which the active constituents are absorbed by a patient. Maceration and decoction are still widely used in situations where the balance of convenience inherent in the “low” technology involved outweighs the lack of precision in such technology in the context of the more expensive pharmaceutical grade production. In the case of macerates and percolates, solvents are removed by evaporation at temperatures below 100° C. and usually below 60° C.

A wide range of processes based on the use of non-aqueous solvents to extract the constituents from plants have been employed. The non-aqueous solvents may be miscible with water or water immiscible and vary in solvating power. Traditionally, ethyl alcohol in various concentrations has been used to extract active substances from plant materials. Tinctures are alcoholic solutions produced in this way and tinctures of plant materials have been used for decades. Where the final concentration of alcohol is greater than approximately 20% by volume, the tincture remains microbiologically stable and such tinctures have been widely used in compounding prescriptions. However, extracting with ethanol pulls out substances such as glycosides, flavonoids and alkaloid salts which are examples of classes of compound known to be biologically active. It also extracts considerable amounts of plant pigments, such as chlorophyll and carotenoids. By using higher alcoholic strengths, lipid-soluble material may be extracted. Tinctures contain less inert material than macerates or decoctions, but are still complex mixtures of plant constituents. Where the presence of alcohol is not required the tincture can be evaporated to produce extracts. Liquid and solid extracts produced in this way are well known.

Moreover, in the so called Butane Hash Oil (BHO) extraction method, butane (a toxic solvent) is used to make a cannabis “red oil” commonly called hash oil, whereby raw cannabis is saturated in butane, which reduces the raw cannabis into an oil that is separated from the plant material. In this method, cooled butane is passed through a dried herbal material under pressure and allowed to expand as it is released from its storage vessel and cools into a liquid with a temperature below 0° C. One advantage of using butane in this manner is that it extracts a high percentage of botanical surface molecules such as cannabinoids. However, because of non-polar solvent properties, butane nonseletively extracts hydrophobic constituents having no known biological activity, such as plant waxes, in addition to desirable components such as terpenes; this is a disadvantage. The extraction of the terpenes could be improved by the maceration (soaking) of the dried botanical source material, however, it is believed that this would yield greater extraction of undesirable plant waxes and pigment molecules such as chlorophyll.

Of course other techniques such as supercritical fluid extraction are known for extracting plant materials, amongst them supercritical fluid CO₂ extraction. Extraction with supercritical fluid CO₂ has been used to remove active constituents from foods such as caffeine from coffee beans, and humulene and other flavours from hops (Humulus lupulus). In the supercritical carbon dioxide (CO₂) extraction method, CO₂ is used as solvent at a temperature and pressure above the critical point, 304.25K and 72.9 atm, respectively as a solvent. Above the critical point, carbon dioxide is a supercritical fluid having the properties midway between that of a gas and a liquid. The process allows for manipulation of solvate power by varying pressure and temperature and by the addition of accessory solvents (modifiers) such as alcohols. The advantage of supercritical extraction is that it can efficiently remove more of the constituent terpenes than the BHO extraction process, and the solvent is much safer to human health for products intended for human consumption. However, when using supercritical CO₂ at temperatures above 0° C., some of the more volatile constituent terpene compounds will be lost, destroyed, or otherwise unobtainable. In addition, because of the high pressures required to achieve a supercritical state CO₂, more of the undesirable plant waxes and chlorophyll will be extracted in the final product. Thus, use of supercritical CO₂ is highly undesirable for selectively extracting constituents in a botanical materal.

In the Quick Wash Isopropyl Alcohol method (QWISO), isopropyl alcohol is utilized at subfreezing (<0° C.) temperatures. However, this method is merely a simple quick wash to dissolve trichomes and their contents from the surface of dried botanical materials, such as the flowers of the cannabis plant. The solvent is then filtered to separate the dissolved target molecules from the spent botanical. The advantage of QWISO is the speed at which target compounds can be extracted from the trichomes of such flowers. The disadvantage is that the speed does not allow for sufficient extraction of the terpenes. Conversely, merely increasing the retention time of the isopropanol with the dried cannabis flowers would result in a high yield of undesirable plant waxes and pigment molecules such as chlorophyll due to the fact that the polar protic nature of the solvent would disrupt cell walls releasing the plant waxes and chlorophyll more readily.

It will also be appreciated that when extracting medicines from a botanical material for the purpose of obtaining a pharmeutically active ingredient, conventional approaches focus on extracting a single active ingredient. In contrast, there is an increasing demand to develop methodologies capable of maintaining the relative amounts of desirable consitutents (compounds) found in a starting botanical material. In particular, it has been found that with some botanical materials (e.g., cannabis, tobacco, etc.) it is more desirable to obtain a substantially “whole plant extract.” As used herein, “whole plant extract” denotes an extract in which all of the desired constituents that are present in a botanical material (e.g., cannabinoids, nictotine, etc.) are extracted together with other plant fractions (e.g. terpenes, etc.) without any undesirable constituents (e.g., chlorophylls, waxes, fats, lipids, etc.). In other words, undesirable constituents are preferably left behind and not extracted. A “whole plant extract” may be formulated into a medicine or used in a smoking or vaporizing article. In some cases, a “whole plant extract” will exhibit an enhanced thereapeutic effect. In other cases, a “whole plant extract” will have an aesthetically pleasing bouquet or aroma of essences that are characteristic of its unadulterated native starting botanical material but without exhibiting any deleterious or otherwise undesirable effects that are experienced when undesirable constituents are still present the extracted botanical material.

It has also been found that certain components of a whole plant extract have the ability to effectively control or modulate the known effect of orther plant components. One example of this action is the exacerbated psychomimetic impact of THC by co-administration of the monoterpene β-myrcene. It is believed that bioactive plant components elicit a synergism determined by the specific mixture or proportion of one or more modulating compounds present in the natural state of the botanical substance, which can result in a characteristic effect. With specific regard to Cannabis spp., this modulation of the known effect of cannabinoids may be desirable and is unique to each Cannabis species. This modulatory effect is commonly referred to as the entourage effect or “strain character” of the plant. Examples of modulating compounds may include, but are not limited to, monoterpenes, diterpenes, sesquiterpenes, flavonoids, and the like.

Accordingly, there is a need to address the aforementioned and/or other problems currently associated with conventional botanical material extraction methods.

### BRIEF DESCRIPTION OF THE DRAWINGS

The accompanying drawings, which are incorporated in and constitute a part of the specification, illustrates the present invention and, together with the description, serve to exemplify the principles of the present invention.

FIG. 1 shows % Yield THC and THCA in resin samples resuspended in EtOH relative to theoretical values in dried cannabis flowers.

FIG. 2 shows % w/w of THCA and THC in cannabis extract resin.

FIG. 3 shows THCA: THC ratio in cannabis extract resin (EtOH samples) and dried cannabis flowers.

FIG. 4 shows % w/w terpenes in cannabis extract resin and dried cannabis flower starting material.

FIG. 5 shows % w/w of all terpene content in extracted cannabis resin resuspended in EtOH.

FIGS. 6 to 12 each show the amount of residual terpenes remaining 1× extracted (spent) dried cannabis flowers (WRB samples).

FIG. 13 shows % w/w of individual terpenes in input dried cannabis flowers and cannabis acetone extracts.

FIGS. 14 and 15 show % w/w amount of terpene and cannabinoid content, respectively, in a cannabis extract sample designated as 198842-1.

FIGS. 16 and 17 show % w/w amount of terpene and cannabinoid content in a cannabis extract sample designated as 198553-2.

FIGS. 18 and 19 show % w/w amount of terpene and cannabinoid content, respectively, in a cannabis extract sample designated as 198842-2.

FIGS. 20 and 21 show % w/w amount of terpene and cannabinoid content, respectively, in a cannabis extract sample designated as 198842-3.

### DESCRIPTION OF EXAMPLE EMBODIMENTS

The example embodiments described herein are believed to address one or more of the previously described or other problems associated with conventional botanical extraction methods whereby selectivity and/or yield of desirable volatile compounds, e.g., terpenes, terpenoids and other essential oils described herein, are deleteriously affected during extraction and/or purification steps.

It will be appreciated that the example embodiments disclosed in this written description relate, in part, to improvements in methods used to extract target compounds from botanical materials. In one example embodiment there is a two solvent extraction method that uses 2-propanone and carbon dioxide (provided by sublimating dry ice in the 2-propanone) to advantageously enhance desirable flavors and aromas in the resulting extract without significantly extracting waxes and pigment molecules that undesirably contaminate the final product and impart a reduced yield, quality, flavour, aroma, etc. The multi-step method can be carried out under various conditions that provide an optimum system for extracting only desirable molecules as well as removing the solvent in an effective manner that significantly reduces the loss of target molecules in the extract.

It is believed that an unexpected and superior advantage of the example embodiments described herein is the ability to extract/isolate at least target compound or profile of target compounds from a botanical material (e.g., cannabinoids, nicotine, aromatic or bioactive terpenes, essences, etc.) without extracting undesirable constituents such as waxes, chlorophyll, fats, lipids, pigments, etc. The resulting extract contains the desired compound(s) in a relatively high degree of purity, substantially free from pigments, chlorophyll, waxes, sterols, fats and other lipid-soluble components which characterize solvent extracts obtained via conventional methods. For instance, with respect to cannabis extracts, it is possible to obtain a high percentage of purity of the extract when comparing free cannabinoid to the corresponding carboxylic acids in extracts produced by other methods. In addition, the methods disclosed herein may provide an extract that is substantially free of inert plant materials and may be of sufficient quality to be processed directly into pharmaceutical dosage forms, if desired. Further, the example embodiments exhibit markedly increased selectivity for extraction of cannabinoids and other volatile compounds found in various botanical materials, thereby producing a terpene-rich extract, if desired.

In an example embodiment, the overall extraction method may be optimized by varying temperature, retention time, pH and/or strength and amount of the 2-propanone co-solvent in order to vary conditions to obtain, for example, a more complete extraction of total cannabinoid content or total terpene content.

It will be understood by all readers of this written description that the example embodiments described herein and claimed hereafter may be suitably practiced in the absence of any recited feature, element or step that is, or is not, specifically disclosed herein. For instance, references in this written description to “one embodiment,” “an embodiment,” “an example embodiment,” and the like, indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one of ordinary skill in the art to affect such feature, structure, or characteristic in connection with other embodiments whether or not explicitly described.

All publications and references cited herein, including those in the Background section, are expressly incorporated herein by reference in their entirety. However, if there are any differences between any similar or identical terms found an incorporated publication or reference and those explicitly put forth or defined in this written description, then those terms definitions or meanings explicitly put forth in this written description shall control in all respects. Further, any reference to prior art in this specification is not, and should not be taken as, an acknowledgment or any form of suggestion that such prior art forms part of the common general knowledge in any country.

No language or terminology in this specification should be construed as indicating any non-claimed element as essential or critical. All methods described herein can be performed in any suitable order unless otherwise indicated herein. The use of any and all examples, or example language (e.g., “such as”) provided herein, is intended merely to better illuminate example embodiments and does not pose a limitation on the scope of the claims appended hereto unless otherwise claimed.

Throughout this specification (i.e., the written description, drawings, claims and abstract), the word “comprise”, or variations such as “comprises” or “comprising,, “including,” “containing,” and the like will be understood to imply the inclusion of a stated element or integer or group of elements or integers but not the exclusion of any other element or integer or group of elements or integers, unless the context requires otherwise.

To facilitate understanding of this example embodiments set forth herein, a number of terms are defined below. Generally, the nomenclature used herein and the laboratory procedures in biology, biochemistry, organic chemistry, medicinal chemistry, pharmacology, etc. described herein are generally well known and commonly employed in the art. Unless defined otherwise, all technical and scientific terms used herein generally have the same meaning as commonly understood in the art to which this disclosure belongs. In the event that there is a plurality of definitions for a term used herein, those in this written description shall prevail unless stated otherwise herein.

As used herein, the singular forms “a,” “an,” and “the” may also refer to plural articles, i.e., “one or more,” “at least one,” “and/or” are open-ended expressions that are both conjunctive and disjunctive in operation. For example, the term “a cannabinoid” includes “one or more cannabinoids”. Further, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. The terms “a” or “an” entity refers to one or more of that entity. As such, the terms “a” (or “an”), “one or more” and “at least one” can be used interchangeably herein.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller subranges are also included. The upper and lower limits of these smaller ranges are also included therein, subject to any specifically excluded limit in the stated range. For example, a range of “about 0.1% to about 5%” or “about 0.1% to 5%” may be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The term “about” or “approximately” means an acceptable error for a particular recited value, which depends in part on how the value is measured or determined. In certain embodiments, “about” can mean 1 or more standard deviations. When the antecedent term “about” is applied to a recited range or value it denotes an approximation within the deviation in the range or value known or expected in the art from the measurements method. For removal of doubt, it shall be understood that any range stated in this written description that does not specifically recite the term “about” before the range or before any value within the stated range inherently includes such term to encompass the approximation within the deviation noted above.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “botanical” and “botanical material” is used herein to denote plants, plant parts (e.g., bark, leaves, stems, roots, flowers, fruits, seeds, berries), plant exudates, algae, and macroscopic fungus, depending on the context.

The term “cannabis” refers to whole cannabis plants and also parts thereof which contain the principal medically active constituents, for example the aerial parts of the plant or isolated leaves and/or flowering heads. The term also encompasses freshly harvested plant material, and also plant material which has been subjected to a pre-treatment step such as, for example, material which has been dried. This includes cannabis material which has been allowed to air dry after harvesting.

“Solvent” is used herein to denote a liquid or gas capable of dissolving a solid or another liquid or gas. Non-limiting examples of solvents include carbon dioxide (CO₂), n-butanol, 2-propanone (acetone), ethanol, acetic acid, isopropanol, n-propanol, methanol, formic acid, 1,4-dioxane, tetrahydrofuran, acetonitrile, dimethylformamide, and dimethyl sulfoxide.

As used herein, “solvent system” refers to one or more solvents that dissolve a solute (a chemically different liquid, solid or gas), resulting in a solution. The maximum quantity of solute that can dissolve in a specific volume of solvent system varies with temperature and pressure. The solvent system can have a specified polarity and proticity. As such, solvent system can be polar, nonpolar, protic, or aprotic, wherein each of these terms is used in a relative manner.

As used herein, “polarity” refers to a separation of electric charge leading to a molecule or its chemical groups having an electric dipole or multipole moment. Polar molecules interact through dipole-dipole intermolecular forces and hydrogen bonds. Molecular polarity is dependent on the difference in electronegativity between atoms in a compound and the asymmetry of the compound's structure. Polarity underlies a number of physical properties including surface tension, solubility, and melting- and boiling-points.

A “protic solvent” is used herein to denote a solvent that contains dissociable H+, for example a hydrogen atom bound to an oxygen atom as in a hydroxyl group or a nitrogen atom as in an amino group. A protic solvent is capable of donating a proton (H+). Conversely, an “aprotic” solvent cannot donate H+.

As used herein, “polar” or “polar solvent” refers to a molecule having a net dipole as a result of the opposing charges (i.e., having partial positive and partial negative charges) from polar bonds arranged asymmetrically. Water (H.sub.2O ) is an example of a polar molecule since it has a slight positive charge on one side and a slight negative charge on the other. The dipoles do not cancel out resulting in a net dipole. Due to the polar nature of the water molecule itself, polar molecules are generally able to dissolve in water. Another example includes sugars (like sucrose), which have many polar oxygen-hydrogen (—OH) groups and are overall highly polar.

As used herein, “nonpolar” or “nonpolar solvent” refers to a molecule having an equal sharing of electrons between the two atoms of a diatomic molecule or because of the symmetrical arrangement of polar bonds in a more complex molecule. For example, the boron trifluoride molecule (BF₃) has a trigonal planar arrangement of three polar bonds at 120°. This results in no overall dipole in the molecule. In methane, the bonds are arranged symmetrically (in a tetrahedral arrangement) so there is no overall dipole. In the methane molecule (CH₄) the four C—H bonds are arranged tetrahedrally around the carbon atom. Each bond has polarity (though not very strong). However, the bonds are arranged symmetrically so there is no overall dipole in the molecule. The diatomic oxygen molecule (O₂) does not have polarity in the covalent bond because of equal electronegativity, hence there is no polarity in the molecule

As used herein, “Cannabis” refers to a genus of flowering plants that includes a single species, Cannabis sativa, which is sometimes divided into two additional species, Cannabis indica and Cannabis ruderalis. These three taxa are indigenous to Central Asia, and South Asia. Cannabis has long been used for fiber (hemp), for seed and seed oils, for medicinal purposes, and as a recreational drug. Various extracts including hashish and hash oil are also produced from the plant. Suitable strains of Cannabis include, e.g., indica-dominant (e.g., Blueberry, BC Bud, Holland's Hope, Kush, Northern Lights, Purple, and White Widow), Pure sativa (e.g., Acapulco Gold and Malawi Gold (Chamba)), and Sativa-dominant (e.g., Charlotte's Web, Diesel, Haze, Jack Herer, Shaman, Skunk, Sour, and Te Puke Thunder). The Cannabis can include any physical part of the plant material, including, e.g., the leaf, bud, flower, trichome, seed, or combination thereof. Likewise, the Cannabis can include any substance physically derived from Cannabis plant material, such as, e.g., kief and hashish.

As used herein, “cannabinoid” refers to a class of diverse chemical compounds that act on cannabinoid receptors on cells that repress neurotransmitter release in the brain. These receptor ligands include the endocannabinoids (produced naturally in the body by humans and animals), the phytocannabinoids (found in Cannabis and some other plants), and synthetic cannabinoids (manufactured chemically). The most notable cannabinoid is the phytocannabinoid Δ9-tetrahydrocannabinol (THC), the primary psychoactive compound of Cannabis. Cannabidiol (CBD) is another major constituent of the plant. CBD-rich strains can yield upwards of 80% CBD in extracted resins using the methods described herein, e.g., it has been made possible to extract a cannabis resin with >70% CBD.

As used herein, “terpene,” “terpenoid” or “isoprenoid” refers to a large and diverse class of naturally occurring organic chemicals similar to terpenes, derived from five-carbon isoprene units assembled and modified in thousands of ways. Most are multicyclic structures that differ from one another not only in functional groups but also in their basic carbon skeletons. These lipids can be found in all classes of living things, and are the largest group of natural products. Plant terpenoids are used extensively for their aromatic qualities. They play a role in traditional herbal remedies and are under investigation for antibacterial, antineoplastic, and other pharmaceutical functions. Terpenoids contribute to the scent of eucalyptus, the flavors of cinnamon, cloves, and ginger, the yellow color in sunflowers, and the red color in tomatoes. Well-known terpenoids include citral, menthol, camphor, salvinorin A in the plant Salvia divinorum, the cannabinoids found in Cannabis, ginkgolide and bilobalide found in Ginkgo biloba, and the curcuminoids found in turmeric and mustard seed.

As used herein, “flavonoid” refers to a class of plant secondary metabolites. Flavonoids were referred to as Vitamin P (probably because of the effect they had on the permeability of vascular capillaries) from the mid-1930s to early 50s, but the term has since fallen out of use. According to the IUPAC nomenclature, they can be classified into: flavonoids or bioflavonoids; isoflavonoids, derived from 3-phenylchromen-4-one (3-phenyl-1,4-benzopyrone) structure; and neoflavonoids, derived from 4-phenylcoumarine (4-phenyl-1,2-benzopyrone) structure.

As used herein, “kief” refers to the resin glands (or trichomes) of Cannabis which may accumulate in containers or be sifted from loose dry Cannabis flower with a mesh screen or sieve. Kief typically contains a much higher concentration of psychoactive cannabinoids, such as THC, than that of the Cannabis flowers from which it is derived. Traditionally, kief has been pressed into cakes of hashish for convenience in storage, but can be vaporized or smoked in either form.

As used herein, “hashish” refers to a Cannabis product composed of compressed or purified preparations of stalked resin glands, called trichomes. It contains the same active ingredients—such as THC and other cannabinoids—but in higher concentrations than unsifted buds or leaves.

As used herein, “leaf” refers to an organ of a vascular plant, as defined in botanical terms, and in particular, in plant morphology. In reference to Cannabis, the first pair of leaves usually have a single leaflet, the number gradually increasing up to a maximum of about thirteen leaflets per leaf (usually seven or nine), depending on variety and growing conditions. At the top of a flowering plant, this number again diminishes to a single leaflet per leaf. The lower leaf pairs usually occur in an opposite leaf arrangement and the upper leaf pairs in an alternate arrangement on the main stem of a mature plant.

As used herein, “bud” refers to a flower-bearing stem or branch of the Cannabis plant, especially a stem or branch bearing a mass of female flowers with associated leaves. The stem or branch bearing the female flowers can be fresh, or can be dried. The pistils of the female Cannabis flower are surrounded by a mass of trichome-rich petals and leaves, and can contain higher concentrations of cannabinoids than do the plant leaves or stems. A bud, e.g., a mass of female flowers and associated leaves, usually covered with trichomes, can be further processed mechanically, i.e., “trimming” or “cleaning” the stem bearing the female flowers by removal of larger leaves and stem material. Buds, and cleaned buds, can be used as a Cannabis plant material in practice of a method of the invention.

As used herein, “trichome” refers to a fine outgrowth or appendage on plants and certain protists. Trichomes are of diverse structure and function. Examples are hairs, glandular hairs, scales, and papillae. In reference to Cannabis, the trichome is a glandular trichome that occurs most abundantly on the floral calyxes and bracts of female plants.

As used herein, “seed” refers to an embryonic plant enclosed in a protective outer covering called the seed coat, usually with some stored food. It is a characteristic of spermatophytes (gymnosperm and angiosperm plants) and the product of the ripened ovule which occurs after fertilization and some growth within the mother plant. The formation of the seed completes the process of reproduction in seed plants (started with the development of flowers and pollination), with the embryo developed from the zygote and the seed coat from the integuments of the ovule.

As used herein, “tincture” refers to a solvent extract of plant or animal material, a solution of such, or of a low volatility substance.

As used herein, “hash oil” refers to a form of Cannabis. It is a resinous matrix of cannabinoids obtained from the Cannabis plant by solvent extraction, formed into a hardened or viscous mass. Hash oil can be the most potent of the main Cannabis products because of its high THC content which can vary depending on the plant.

As used herein, “concentrate” or “essential oil” refers to a substance obtained by extracting a raw material, using a solvent, wherein the solvent has substantially been removed.

The example embodiments disclosed herein are based, in part, on an unpredicted/unexpected discovery that 2-propanone (acetone) with or without the presence of subcritical CO₂, under certain conditions described herein, may be used to selectively extract target compounds from botanical materials even though the use of 2-propanone has been avoided in conventional botanical extraction scenarios due to its strong polar (aprotic) nature and its unfavorable capability to indiscriminately remove undesirable amounts of plant wax and chlorophyll from botanical material.

According to the example embodiments, it will be appreciated that it is not necessary to use strong solvation (e.g., the use of supercritical conditions or strong solvents) to extract the desirable constituents of a botanical source material to obtain a whole plant extract of constituents having a profile relative to each that is substantially similar to the profile of the desirable constituents found in the naturally occruing botanical source material without undesirable waxes or chlorophyll. Thus, an advantage of the example embodiments is that a simpler and cheaper process has been achieved without the need for complex cleanup steps or further downstream extraction steps or solvents.

Other advantages of certain embodiments are disclosed below or may be realized and appreciated while practicing one or more example embodiments. The following are certain aspects of the example embodiments further described herein.

A method for producing an extract from a botanical material, wherein the extract contains at least one target compound, the method comprising:
admixing the botanical material with acetone to obtain a mixture;
optionally adding dry ice to the mixture;
allowing the temperature of the mixture to reach about −78.5° C. to 0° C.;
optionally agitating the mixture;
filtering the mixture to remove to obtain a filtrate; and
removing the solvent from the filtrate to obtain the extract.

In a further example embodiment, there is provided a method for producing an extract from a botanical material, wherein the extract contains at least one target compound or a preferred profile of various target compounds or constituents, the method comprising, consisting essentially of, or consisting of:
adding to a vessel (pressurized or unpressurized) the botanical material and a solvent comprising, consisting essentially of, or consisting of 2-propanone (acetone) and dry ice to obtain a mixture;
allowing the temperature of the mixture in the vessel to reach about −40° C. or less;
optionally agitating the mixture to optimize contact of botanical material with the carbon dioxide gas sublimating/eluting from the dry ice and the acetone co-solvent;
filtering the mixture to remove particle sizes of at least 100 microns to obtain a filtrate;
removing the solvent from the filtrate to obtain the extract.

The method as described above, wherein the botanical material is selected from a member of the group consisting of plants, plant parts (e.g., bark, leaves, stems, roots, flowers, fruits, seeds, nuts, berries), macerated or comminuted plant parts, plant exudates, and mixtures thereof.

The method as described above, wherein the vessel comprises stainless steel or glass.

The method as described above, wherein the amount (w/w) of 2-propanone to botanical material that is present in the vessel is about 15:1, 10:1, 5:1, 4:1, 3:1, 2:1, 1:1, 1:2, 1:3, 1:4, 1:5, 1:10, 1:15 and all sub ranges therebetween.

The method as described above, wherein the amount (w/w) of dry ice to botanical material present in the vessel is about 15:1, 10:1, 5:1, 4:1, 3:1, 2:1, 1:1, 1:2, 1:3, 1:4, 1:5, 1:10, 1:15 and all sub ranges therebetween.

The method as described above, wherein the agitating step is performed for 1 minute to 360 minutes or more, it being understood that shorter times will produce a lower yield, but higher purity, of cannabinoids and a lower yield of the terpenes, waxes, pigment molecules (e.g., chlorphyll), etc.

The method as described above, wherein the dry ice and 2-propanone are added to the vessel prior to adding the botanical material.

The method as describesd above, wherein the botanical material is added to the vessel prior to adding the dry ice and 2-propanone.

The method as described above, wherein the dry ice is added to the vessel prior to the botanical material and 2-propanone.

The method as described above, wherein the dry ice is not added to the vessel.

The method as described above, wherein the pressure in the vessel is maintained at about 1 bar.

The method as described above, wherein the vessel is configured to be pressurized in an amount of from about 1 bar to about 50 bar, and all sub ranges therebetween, using any suitable means including.

The method as described above, wherein the temperature of the mixture in the vessel is maintained at about −78.5° C., using any suitable means including, without limitation, controlling the amount of dry ice and/or 2-propanone in the vessel, ice bath, refrigerated jacket or column, etc.

The method as described above, wherein the temperature of the mixture in the vessel is maintained at about −76° C., using any suitable means including, without limitation, controlling the amount of dry ice and/or 2-propanone in the vessel, ice bath, refrigerated jacket or column, etc.

The method as described above, wherein during the agitating step, the temperature of the mixture in the vessel is maintained between −78° C. and −20° C. and all sub ranges therebetween, using any suitable means including, without limitation, controlling the amount of dry ice and/or 2-propanone in the vessel, ice bath, refrigerated jacket or column, etc.

The method as described above, wherein during the agitating step, the temperature of the mixture in the vessel is maintained between −78° C. and −10° C. and all sub ranges therebetween, using any suitable means including, without limitation, controlling the amount of dry ice and/or 2-propanone in the vessel, ice bath, refrigerated jacket or column, etc.

The method as described above, wherein during the agitating step, the temperature of the mixture in the vessel is maintained at about −78° C. and 0° C., and all sub ranges therebetween, using any suitable means including, without limitation, controlling the amount of dry ice and/or 2-propanone in the vessel, ice bath, refrigerated jacket or column, etc.

The method as described above, wherein the filtering step is performed at a temperature of between −78° C. and 0° C., and all sub ranges therebetween.

The method as described above, wherein the temperature of the mixture in the vessel is maintained at about 0° C., using any suitable means including, without limitation, controlling the amount of dry ice and/or 2-propanone in the vessel, bath, refrigerated jacket or column, etc.

The method as described above, wherein the solvent is removed from the filtrate by applying heat to the vessel at a temperature of about 60° C. or less.

The method as described above, wherein the solvent is removed from the filtrate by applying a vacuum.

The method as described above, wherein the extract comprises at least one compound selected from a member of the group consisting of terpenes, terpenoids, cannabinoids, alkaloids and mixtures thereof.

The method as described above, wherein the compound is isolated or purified from the extract by any suitable means including without limitation wiped film, distillation, flash chromatography, medium pressure liquid chromatography, high performance liquid chromatography (HPLC), distillation, sublimation, etc.

The method as described above, wherein the filtrate is a liquid or semi-solid.

The method as described above, wherein the botanical material has a high CBD content relative to THC content.

The method as described above, wherein the botanical material has a high THC content relative to CBD content.

An extract (including whole-plant extracts) obtained by an example embodiment as described herein.

A container comprising, consisting essentially of, or consisting of an extract (including whole-plant extracts) obtained by an example embodiment, as described herein.

A pharmaceutical composition, dietary supplement or food item, comprising, consisting essentially of, or consisting of an extract obtained by a method as described above and a therapeutically acceptable or inert carrier.

Without being bound by any theory of operation, it has been found that the nature of 2-propanone imparts an inability for 2-propanone to hydrogen bond with itself, yet retaining the ability to act as a hydrogen bond recipient in order to bond other species, thereby aiding in extraction. In accordance with an example embodiment, the high dipole moment of 2-propanone allows it to be desirable in extracting high quantities of target compounds contained in botanical materials, but with a lower dipole moment than a solvent like DMSO, which is known to be effective at extracting most components of a botanical. It has been found that polar aprotic solvents do not participate in chemical reactions that adversely affect yield, which leads to increased yields or more completely extracted material, some of which would be undesirable (in most cases such as, but not limited to, wax or chlorophyll). The physical properties of 2-propanone as an undesirably polar solvent do not allow one to predict that the resulting product of a botanical extraction method is unexpected as the temperature conditions of the reaction combined with the presence of a non-polar co-solvent significantly reduces the amount of undesired pigment and wax molecules that are removed from the botanical of interest. Thus, by utilizing 2-propanone in this way, one is able to control the amount of the various reactants in order to selectively extract the terpenes and terpenoids (e.g., cannabinoids), which are relatively more volatile and easily lost during conventional extraction processes using temperatures above −20° C., −10° C., 0° C. or more.

Cannabis contains about 100 compounds believed to be responsible for, in part, a distinctive characteristic aroma. These compounds are mainly volatile compounds, such as terpenes, and sesquiterpenes. The predominately volatile compounds present in cannabis, which may be extracted using the methods disclosed herein, include α-Pinene, Myrcene, Linalool, Limonene, Trans-β-ocimene, α-Terpinolene, Trans-caryophyllene, α-Humulene, and Caryophyllene-oxide. Cannabis sativa contains about 61 compounds belonging to the class of cannabinoids. These are lipophilic, nitrogen-free, mostly phenolic compounds. The neutral cannabinoids are biogenetically derived from a monoterpene and a phenol, the acidic cannabinoids from a monoterpene and a phenolic acid. Among the most important cannabinoids there are, for example:
Δ9-tetrahydrocannabinol Δ9-THC
Δ8-tetrahydrocannabinol Δ8-THC
cannabichromene CBC
cannabidiol CBD
cannabigerol CBG
cannabinidiol CBND
cannabinol CBN

These volatile compounds may be isolated or purified from the extract obtained by an example embodiment describe herein this writtend description.

In general, examples of the volatile compounds that may be extracted from botanical materials that are subjected to the extraction methods disclosed herein include, but are not limited to, members selected from the group consisting of: α- or β-pinene; α-campholenic aldehyde; α-citronellol; α-iso-amyl-cinnamic (e.g., amyl cinnamic aldehyde); α-pinene oxide; α-cinnamic terpinene; α-terpineol (e.g., 1-methyl-4-isopropyl-1-cyclohexen-8-ol; λ-terpinene; achillea; aldehyde C16 (pure); alpha-phellandrene; amyl cinnamic aldehyde; amyl salicylate; anethole; anise; aniseed; anisic aldehyde; basil; bay; benzyl acetate; benzyl alcohol; bergamot (e.g., Monardia fistulosa, Monarda didyma, Citrus bergamia, Monarda punctata); bitter orange peel; black pepper; white pepper; borneol; calamus; camphor; cananga oil (e.g., java); cardamom; carnation (e.g., dianthus caryophyllus); carvacrol; carveol; cassia; castor; cedar (e.g., hinoki); cedarwood; chamomile; cineole; cinnamaldehyde; cinnamic alcohol; cinnamon; cis-pinane; citral (e.g., 3, 7-dimethyl-2, 6-octadienal); citronella; citronellal; citronellol dextro (e.g., 3-7-dimethyl-6-octen-1-ol); citronellol; citronellyl acetate; citronellyl nitrile; citrus unshiu; clary sage; clove (e.g., eugenia caryophyllus); clove bud; coriander; corn; cotton seed; 6-tert-butyl-m-cresol; d-dihydrocarvone; decyl aldehyde; diethyl phthalate; dihydroanethole; dihydrocarveol; dihydrocarvacrol; dihydrolinalool; dihydromyrcene; dihydromyrcenol; dihydromyrcenyl acetate; dihydroterpineol; dimethyl salicylate; dimethyloctanal; dimethyloctanol; dimethyloctanyl acetate; diphenyl oxide; dipropylene glycol; d-limonene; d-pulegone; estragole; ethyl vanillin (e.g., 3-ethoxy-4-hydrobenzaldehyde); eucalyptol (e.g., cineole); eucalyptus citriodora; eucalyptus globulus; eucalyptus; eugenol (e.g., 2-methoxy-4-allyl phenol); evening primrose; fenchol; fennel; ferniol.tm.; fish; florazon (e.g., 4-ethyl-α, α-dimethyl-benzenepropanal); galaxolide; geraniol (e.g., 2-trans-3, 7-dimethyl-2, 6-octadien-8-ol); geraniol; geranium; geranyl acetate; geranyl nitrile; ginger; grapefruit; guaiacol; guaiacwood; gurjun balsam; heliotropin; herbanate (e.g., 3-(1-methyl-ethyl) bicyclo (2, 2, 1) hept-5-ene-2-carboxylic acid ethyl ester); hiba; hydroxycitronellal; i-carvone; i-methyl acetate; ionone; isobutyl quinoleine (e.g., 6-secondary butyl quinoline); isobornyl acetate; isobornyl methylether; isoeugenol; isolongifolene; jasmine; jojoba; juniper berry; lavender; lavandin; lemon grass; lemon; lime; limonene; linallol oxide; linallol; linalool; linalyl acetate; linseed; litsea cubeba; I-methyl acetate; longifolene; mandarin; mentha; menthane hydroperoxide; menthol crystals; menthol laevo (e.g., 5-methyl-2-isopropyl cyclohexanol); menthol; menthone laevo (e.g., 4-isopropyl-1-methyl cyclohexan-3-one); methyl anthranilate; methyl cedryl ketone; methyl chavicol; methyl hexyl ether; methyl ionone; mineral; mint; musk ambrette; musk ketone; musk xylol; mustard (also known as allylisothio-cyanate); myrcene; nerol; neryl acetate; nonyl aldehyde; nutmeg (e.g., myristica fragrans); orange (e.g., citrus aurantium dulcis); orris (e.g., iris florentina) root; para-cymene; para-hydroxy phenyl butanone crystals (e.g., 4-(4-hydroxyphenyl)-2-butanone); passion palmarosa oil (e.g., cymbopogon martini); patchouli (e.g., pogostemon cablin); p-cymene; pennyroyal oil; pepper; peppermint (e.g., mentha piperita); perillaldehyde; petitgrain (e.g., citrus aurantium amara); phenyl ethyl alcohol; phenyl ethyl propionate; phenyl ethyl-2-methylbutyrate; pimento berry; pimento leaf; pinane hydroperoxide; pinanol; pine ester; pine needle; pine; pinene; piperonal; piperonyl acetate; piperonyl alcohol; plinol; plinyl acetate; pseudo ionone; rhodinol; rhodinyl acetate; rosalin; rose; rosemary (e.g., rosmarinus officinalis);) ryu; sage; sandalwood (e.g., santalum album); sandenol; sassafras; sesame; soybean; spearmint; spice; spike lavender; spirantol; starflower; tangerine; tea seed; tea tree; terpenoid; terpineol; terpinolene; terpinyl acetate; tert-butylcyclohexyl acetate; tetrahydrolinalool; tetrahydrolinalyl acetate; tetrahydromyrcenol; thulasi; thyme; thymol; tomato; trans-2-hexenol; trans-anethole and metabolites thereof; turmeric; turpentine; vanillin (e.g., 4-hydroxy-3-methoxy benzaldehyde); vetiver; vitalizair; white cedar; white grapefruit; wintergreen (e.g., methyl salicylate), derivatives thereof, and mixtures thereof.

In another example embodiment, a quantity of wet or dried botanical material may be prepared before adding to the vessel by grinding or otherwise, comminuting the whole plant, roots, stems, flowers, and leaves to enhance total yield. To enhance purity of the extract, the botanical may be comminuted and/or macerated to various particle sizes it being understood that the larger the particle size of botanical material, the lower the yield of wax and chlorophyll will be observed, whereas the finer the grind the more of each desired target compound will be obtained in the final extract.

In a further example embodiment, the ratio of solvents to each other and to the amount of botanical material in the vessel may be varied to increase or lower the retention times, which shall be defined as the amount of time that the botanical material is in contact with the solvent system. The ratio of the solvents determines the operating temperature and therefore the relative extraction of terpenes versus waxes versus percentage yield of each. Lower temperatures will restrict the extraction of waxes, but also of a higher yield of total desired cannabinoids and terpenes. If one desires to complete the main extraction in one step, one balances these parameters in any suitable way to obtain the desired extract composition.

In another example embodiment, extraction method is carried out using two steps. A first step “pulls out” the majority of the cannabinoids and highly volatile terpenes. A second extraction pulls out the majority of the balance of the cannabinoids and terpenes, but also some of the undesirable wax and chlorophyll.

In a further example embodiment, the steps comprise:
obtaining dried botanical and charge a round bottom flask with the filtrate from the extraction process.
attaching the round bottom flask to a reflux condenser with cold water circulation at ambient pressure.
heating the flask to extract the 2-propanone at or near 57° C. with refluxing in the cooled condenser. Watch for the temperature to begin rising above 57° C., once it does, allow the temperature to rise slowly to ˜105° C. Maintain 105° C. for 45 minutes and turn off the heat.

Below is a step-wise example embodiment of a two-solvent 2-propanone—dry ice (CO₂) extraction method:
Measure out a certain mass of dried or fresh botanical material (e.g., cannabis herb or other botanical).
In a stainless or glass vessel, measure out a mass of 2-propanone at least about 4-fold that of the botanical material for extraction.
Add an amount of dry ice to the 2-propanone-containing vessel equal to the mass of botanical material to be extracted.
Allow the temperature to equilibrate to about ˜40° C.
Grind and/or Macerate the herb to a desired particle size.
Add the macerated herb to the solvent.
Incubate the extraction mixture with gentle agitation at or below ˜40° C. for at least 10 minutes.
Filter the extraction mixture to remove all solid particles of 100 microns or larger.
Place the extraction mixture in a round-bottom flask to remove the solvent by either:
Applying gentle heating to a maximum of 60° C. with an attached water-jacketed refluxing column open to the environment (open system)
Attaching the round-bottom flask to a rotary evaporator device; apply a vacuum to reduce the pressure and heat to a minimum temperature appropriate to evaporate off the solvent mixture (boiling point of 2-propanone at a given pressure) (closed system).
The final product will be a liquid or semi-solid containing a mixture of desirable volatile compounds and other extract products. Further processes can be performed this extract to produce a purified or fractionated end product.

The method as described above, wherein the botanical material or plant is selected, without limitation, from a member of the group consisting of cannabis, hemp, hops, or tobacco.

The method as described above, may be used to treat, process or obtain extracts from botanical materials/flowering plants (Angiosperms family) selected from a member of the group consisting of Acanthaceae; Achariaceae; Achatocarpaceae; Acoraceae; Actinidiaceae; Adoxaceae; Aextoxicaceae; Aizoaceae; Akaniaceae; Alismataceae; Alseuosmiaceae; Alstroemeriaceae; Altingiaceae; Amaranthaceae; Amaryllidaceae; Amborellaceae; Anacampserotaceae; Anacardiaceae; Anarthriaceae; Ancistrocladaceae; Anisophylleaceae; Annonaceae; Aphanopetalaceae; Aphloiaceae; Apiaceae; Apocynaceae; Apodanthaceae; Aponogetonaceae; Aquifoliaceae; Araceae; Araliaceae; Arecaceae; Argophyllaceae; Aristolochiaceae; Asparagaceae; Asteliaceae; Asteropeiaceae; Atherospermataceae; Austrobaileyaceae; Balanopaceae; Balanophoraceae; Balsaminaceae; Barbeuiaceae; Barbeyaceae; Basellaceae; Bataceae; Begoniaceae; Berberidaceae; Berberidopsidaceae; Betulaceae; Biebersteiniaceae; Bignoniaceae; Bixaceae; Blandfordiaceae; Bonnetiaceae; Boraginaceae; Boryaceae; Brassicaceae; Bromeliaceae; Brunelliaceae; Bruniaceae; Burmanniaceae; Burseraceae; Butomaceae; Buxaceae; Byblidaceae; Cabombaceae; Cactaceae; Calceolariaceae; Calophyllaceae; Calycanthaceae; Calyceraceae; Campanulaceae; Campynemataceae; Canellaceae; Cannabaceae; Cannaceae; Capparaceae; Caprifoliaceae; Cardiopteridaceae; Caricaceae; Carlemanniaceae; Caryocaraceae; Caryophyllaceae; Casuarinaceae; Celastraceae; Centrolepidaceae; Centroplacaceae; Cephalotaceae; Ceratophyllaceae; Cercidiphyllaceae; Chloranthaceae; Chrysobalanaceae; Circaeasteraceae; Cistaceae; Cleomaceae; Clethraceae; Clusiaceae; Colchicaceae; Columelliaceae; Combretaceae; Commelinaceae; Compositae; Connaraceae; Convolvulaceae; Coriariaceae; Cornaceae; Corsiaceae; Corynocarpaceae; Costaceae; Crassulaceae; Crossosomataceae; Ctenolophonaceae; Cucurbitaceae; Cunoniaceae; Curtisiaceae; Cyclanthaceae; Cymodoceaceae; Cynomoriaceae; Cyperaceae; Cyrillaceae; Cytinaceae; Daphniphyllaceae; Dasypogonaceae; Datiscaceae; Degeneriaceae; Diapensiaceae; Dichapetalaceae; Didiereaceae; Dilleniaceae; Dioncophyllaceae; Dioscoreaceae; Dipentodontaceae; Dipterocarpaceae; Dirachmaceae; Doryanthaceae; Droseraceae; Drosophyllaceae; Ebenaceae; Ecdeiocoleaceae; Elaeagnaceae; Elaeocarpaceae; Elatinaceae; Emblingiaceae; Ericaceae; Eriocaulaceae; Erythroxylaceae; Escalloniaceae; Eucommiaceae; Euphorbiaceae; Euphroniaceae; Eupomatiaceae; Eupteleaceae; Fagaceae; Flacourtiaceae; Flagellariaceae; Fouquieriaceae; Frankeniaceae; Garryaceae; Geissolomataceae; Gelsemiaceae; Gentianaceae; Geraniaceae; Gerrardinaceae; Gesneriaceae; Gisekiaceae; Gomortegaceae; Goodeniaceae; Goupiaceae; Grossulariaceae; Grubbiaceae; Guamatelaceae; Gunneraceae; Gyrostemonaceae; Haemodoraceae; Halophytaceae; Haloragaceae; Hamamelidaceae; Hanguanaceae; Haptanthaceae; Heliconiaceae; Helwingiaceae; Hernandiaceae; Himantandraceae; Huaceae; Humiriaceae; Hydatellaceae; Hydnoraceae; Hydrangeaceae; Hydrocharitaceae; Hydroleaceae; Hydrostachyaceae; Hypericaceae; Hypoxidaceae; Icacinaceae; Iridaceae; Irvingiaceae; Iteaceae; Ixioliriaceae; Ixonanthaceae; Joinvilleaceae; Juglandaceae; Juncaceae; Juncaginaceae; Kirkiaceae; Koeberliniaceae; Krameriaceae; Lacistemataceae; Lactoridaceae; Lamiaceae; Lanariaceae; Lardizabalaceae; Lauraceae; Lecythidaceae; Leguminosae; Lentibulariaceae; Lepidobotryaceae; Liliaceae; Limeaceae; Limnanthaceae; Linaceae; Linderniaceae; Loasaceae; Loganiaceae; Lophiocarpaceae; Lophopyxidaceae; Loranthaceae; Lowiaceae; Lythraceae; Magnoliaceae; Malpighiaceae; Malvaceae; Marantaceae; Marcgraviaceae; Martyniaceae; Mayacaceae; Melanthiaceae; Melastomataceae; Meliaceae; Melianthaceae; Menispermaceae; Menyanthaceae; Metteniusaceae; Misodendraceae; Mitrastemonaceae; Molluginaceae; Monimiaceae; Montiaceae; Montiniaceae; Moraceae; Moringaceae; Muntingiaceae; Musaceae; Myodocarpaceae; Myricaceae; Myristicaceae; Myrothamnaceae; Myrtaceae; Nartheciaceae; Nelumbonaceae; Nepenthaceae; Neuradaceae; Nitrariaceae; Nothofagaceae; Nyctaginaceae; Nymphaeaceae; Ochnaceae; Olacaceae; Oleaceae; Onagraceae; Oncothecaceae; Opiliaceae; Orchidaceae; Orobanchaceae; Oxalidaceae; Paeoniaceae; Pandaceae; Pandanaceae; Papaveraceae; Paracryphiaceae; Passifloraceae; Paulowniaceae; Pedaliaceae; Penaeaceae; Pennantiaceae; Pentadiplandraceae; Pentaphragmataceae; Pentaphylacaceae; Penthoraceae; Peraceae; Peridiscaceae; Petenaeaceae; Petermanniaceae; Petrosaviaceae; Phellinaceae; Philesiaceae; Philydraceae; Phrymaceae; Phyllanthaceae; Phyllonomaceae; Physenaceae; Phytolaccaceae; Picramniaceae; Picrodendraceae; Piperaceae; Pittosporaceae; Plantaginaceae; Platanaceae; Plocospermataceae; Plumbaginaceae; Poaceae; Podostemaceae; Polemoniaceae; Polygalaceae; Polygonaceae; Pontederiaceae; Portulacaceae; Posidoniaceae; Potamogetonaceae; Primulaceae; Proteaceae; Putranjivaceae; Quillajaceae; Rafflesiaceae; Ranunculaceae; Rapateaceae; Resedaceae; Restionaceae; Rhabdodendraceae; Rhamnaceae; Rhipogonaceae; Rhizophoraceae; Roridulaceae; Rosaceae; Rousseaceae; Rubiaceae; Ruppiaceae; Rutaceae; Sabiaceae; Salicaceae; Salvadoraceae; Santalaceae; Sapindaceae; Sapotaceae; Sarcobataceae; Sarcolaenaceae; Sarraceniaceae; Saururaceae; Saxifragaceae; Scheuchzeriaceae; Schisandraceae; Schlegeliaceae; Schoepfiaceae; Scrophulariaceae; Setchellanthaceae; Simaroubaceae; Simmondsiaceae; Siparunaceae; Sladeniaceae; Smilacaceae; Solanaceae; Sphaerosepalaceae; Sphenocleaceae; Stachyuraceae; Staphyleaceae; Stegnospermataceae; Stemonaceae; Stemonuraceae; Stilbaceae; Strasburgeriaceae; Strelitziaceae; Stylidiaceae; Styracaceae; Surianaceae; Symplocaceae; Talinaceae; Tamaricaceae; Tapisciaceae; Tecophilaeaceae; Tetrachondraceae; Tetramelaceae; Tetrameristaceae; Theaceae; Thomandersiaceae; Thumiaceae; Thymelaeaceae; Ticodendraceae; Tofieldiaceae; Torricelliaceae; Tovariaceae; Trigoniaceae; Trimeniaceae; Triuridaceae; Trochodendraceae; Tropaeolaceae; Typhaceae; Ulmaceae; Urticaceae; Vahliaceae; Velloziaceae; Verbenaceae; Violaceae; Vitaceae; Vivianiaceae; Vochysiaceae; Winteraceae; Xanthorrhoeaceae; Xeronemataceae; Xyridaceae; Zingiberaceae; Zosteraceae; Zygophyllaceae; and all sub genera, lower classification and species thereof, including, without limitation those listed elsewhere in this written description.

The following are further non-limiting examples of the botanical gernera that may be subjected to example methods described herein.

FAMILY: SOLANACEAE; GENERA: Acnistus; Anisodus; Anthocercis; Anthotroche; Archihyoscyamus; Archiphysalis; Athenaea; Atrichodendron; Atropa; Atropanthe; Aureliana; Benthamiella; Bouchetia; Brachistus; Browallia; Brugmansia; Brunfelsia; Calibrachoa; Capsicum; Cestrum; Chamaesaracha; Coeloneurum; Combera; Crenidium; Cuatresia; Cyphanthera; Cyphomandra; Datura; Deprea; Discopodium; Duboisia; Duckeodendron; Dunalia; Eriolarynx; Espadaea; Exodeconus; Fabiana; Goetzea; Grabowskia; Grammosolen; Habrothamnus; Hawkesiophyton; Herschelia; Hunzikeria; Hyoscyamus; Iochroma; Jaborosa; Jaltomata; Juanulloa; Larnax; Latua; Lehmannia; Leptoglossis; Leucophysalis; Lycianthes; Lycium; Lycopersicon; Lycopersicum; Mandragora; Markea; Melananthus; Merinthopodium; Meyenia; Nectouxia; Nicandra; Nicotiana; Nierembergia; Nolana; Normania; Nycterium; Pantacantha; Petunia; Phrodus; Physaliastrum; Physalis; Physochlaina; Pionandra; Plowmania; Protoschwenkia; Przewalskia; Quincula; Rahowardiana; Reyesia; Salpichroa; Salpichroma; Salpiglossis; Saracha; Schizanthus; Schultesianthus; Schwenckia; Schwenkia; Sclerophylax; Scopolia; Sessea; Solandra; Solanum; Streptosolen; Swartsia; Trianaea; Tubocapsicum; Tzeltalia; Vassobia; Vestia; Withania; Witheringia.

FAMILY: COMPOSITAE; GENERA: Aaronsohnia; Abasoloa; Abrotanella; Absinthium; Acamptopappus; Acanthocephalus; Acanthocladium; Acanthodesmos; Acanthospermum; Acanthostyles; Acanthoxanthium; Acarna; Acarphaea; Achaetogeron; Achillea; Achnophora; Achnopogon; Achyrachaena; Achyrocline; Achyropappus; Achyrophorus; Acilepidopsis; Acilepis; Acmella; Acomis; Acosta; Acourtia; Acrisione; Acritopappus; Acrocentron; Acroclinium; Actinea; Actinella; Actinobole; Actinolepis; Actinomeris; Actinoseris; Actinospermum; Addisonia; Adelostigma; Adenachaena; Adenanthellum; Adenocaulon; Adenocritonia; Adenoglossa; Adenoon; Adenopappus; Adenophyllum; Adenosolen; Adenostemma; Adenostyles; Adenostylium; Adenothamnus; Adopogon; Aedesia; Aegialophila; Aequatorium; Aetheolaena; Aetheopappus; Aetheorhiza; Aganippea; Agathaea; Agathyrsus; Ageratella; Ageratina; Ageratinastrum; Ageratum; Agiabampoa; Agnorhiza; Agoseris; Agrianthus; Agriphyllum; Aimorra; Ainsliaea; Ainsliea; Ajania; Ajaniopsis; Akeassia; Alatoseta; Albertinia; Alboviodoxa; Aldama; Alepidocline; Alfredia; Aliconia; Aliella; Alkibias; Allagopappus; Allardia; Allittia; Allocarpus; Allocephalus; Alloispermum; Allopterigeron; Almutaster; Alomia; Alomiella; Alvordia; Amauria; Amauriopsis; Ambassa; Amberboa; Amblyocarpum; Amblyolepis; Amblyopappus; Amblyopogon; Amblysperma; Amboroa; Ambrosia; Ameghinoa; Amellus; Ammanthus; Ammobium; Ammoseris; Amolinia; Ampelaster; Ampherephis; Amphiachyris; Amphidoxa; Amphiglossa; Amphipappus; Amphirhapis; Amphoricarpos; Anacantha; Anacyclus; Anandria; Anaphalioides; Anaphalis; Anastraphia; Anaxeton; Ancathia; Ancistrocarphus; Anderbergia; Andryala; Anemocarpa; Angeldiazia; Angelphytum; Angianthus; Anisocarpus; Anisochaeta; Anisocoma; Anisopappus; Anisothrix; Anomostephium; Antennaria; Anteremanthus; ×Anthechamomilla; ×Anthechrysanthemum; ×Anthematricaria; ×Anthemimatricaria; Anthemiopsis; Anthemis; Antheropeas; Anthocerastes; Antillanthus; Antillia; Antiphiona; Antithrixia; Antrospermum; Anura; Anvillea; Aostea; Apalochlamys; Apargia; Aphanactis; Aphanopappus; Aphanostephus; Aphelexis; Aphyllocladus; Aplopappus; Aplotaxis; Apodocephala; Apogon; Apopyros; Aposeris; Apostates; Arachnopogon; Aracium; Arbelaezaster; Archibaccharis; Archiserratula; Arctanthemum; Arctium; Arctogeron; Arctotheca; Arctotis; Argentipallium; Argyranthemum; ×Argyrautia; Argyrautia; Argyrocome; Argyroglottis; Argyrophyton; Argyrotegium; Argyroxiphium; Arida; Aristeguietia; Armania; Amaldoa; Arnica; Arnicastrum; Amoglossum; Arnoseris; Aronicum; Arrhenechthites; Arrojadocharis; Arrowsmithia; Artanacetum; Artemisia; Artemisiella; Artemisiopsis; Asanthus; Ascaricida; Ascidiogyne; Askellia; Aspilia; Asplundianthus; Aster; ×Asterago; Asteridea; Asterigeron; Asteriscium; Asteriscus; Asteromoea; Asteropsis; Asterosperma; Asterothamnus; Astranthium; Athanasia; Atherotoma; Athrixia; Athroisma; Atractylis; Atractylodes; Atrichantha; Atrichoseris; Auchera; Aucklandia; Austrobrickellia; Austrocritonia; Austroeupatorium; Austrosynotis; Avellara; Axiniphyllum; Ayapana; Ayapanopsis; Aylacophora; Aynia; Aztecaster; Bacasia; Baccharidastrum; Baccharidiopsis; Baccharis; Baccharodes; Baccharoides; Badilloa; Baeria; Baeriopsis; Bafutia; Bahia; Bahianthus; Bahiopsis; Baileya; Baillieria; Bajacalia; Balbisia; Balduina; Balsamita; Balsamorhiza; Baltimora; Barkhausia; Barkleyanthus; Barnadesia; Barroetea; Barrosoa; Bartlettia; Bartlettina; Basedowia; Batopilasia; Bebbia; Bechium; Bedfordia; Bejaranoa; Bellida; Bellidastrum; Bellidiastrum; Belliopsis; Beffis; Bellium; Belloa; Bembycodium; Benitoa; Berardia; Berhardia; Berkheya; Berkheyopsis; Berlandiera; Berroa; Berthelotia; Berylsimpsonia; Bethencourtia; Bidens; Bigelowia; Billya; Biotia; Bishopalea; Bishopanthus; Bishopiella; Bishovia; Blainvillea; Blakeanthus; Blakiella; Blanchea; Blanchetia; Blennosperma; Blennospora; Blepharipappus; Blepharispermum; Blepharizonia; Blumea; Blumeopsis; Boeberastrum; Boeberoides; Bolandia; Bolanosa; Bolocephalus; Bolophyta; Boltonia; Bombycilaena; Borrichia; Bothriocline; Brachanthemum; Brachionostylum; Brachyactis; Brachyandra; Brachychaeta; Brachyclados; Brachycome; Brachyglottis; Brachylaena; Brachyrhynchos; Brachyris; Brachyscome; Brachythrix; Bradburia; Brasilia; Breea; Brenandendron; Breteuillia; Brickellia; Brickelliastrum; Brintonia; Brocchia; Broteroa; Bryomorphe; Bulbostylis; Bunioseris; Buphthalmum; Burkartia; Caatinganthus; Cabobanthus; Cabreriella; Cacalia; Cacaliopsis; Cacosmia; Cadiscus; Caelestina; Caesulia; Calais; Calanticaria; Calcitrapa; Calea; Calendula; Calimeris; Callicephalus; Callichroa; Callicornia; Callilepis; Calliopsis; Callistephus; Calocephalus; Calomeria; Calopappus; Calorezia; Calostephane; Calotesta; Calotis; Calycadenia; Calycoseris; Calyptocarpus; Camchaya; Campovassouria; Camptacra; Campuloclinium; Campylotheca; Canadanthus; Cancrinia; Cancriniella; Capelio; Caradesia; Caraea; Carbeni; Cardopatium; Carduncellus; Carduocirsium; ×Carduocirsium; ×Carduogalactites; Carduus; Carlina; Carlquistia; Carmelita; Carminatia; Carpesium; Carphephorus; Carphochaete; Carramboa; Carterothamnus; Carthamus; Cassinia; Castalis; Castanedia; Castrilanthemum; Castroviejoa; Catamixis; Catananche; Catatia; Catolesia; Caucasalia; Cavalcantia; Cavea; Caxamarca; ×Celmearia; Celmisia; Cenia; Cenocline; Centaurea; Centaureopappus; Centaurodendron; Centauropsis; Centaurothamnus; ×Centauserratula; Centipeda; Centrachena; Centrapalus; Centratherum; Centrocarpha; Centromadia; Centrospermum; Cephalipterum; Cephalopappus; Cephalophora; Cephalorrhynchus; Cephalosorus; Ceratogyne; Cercostylos; Ceruana; Chacoa; Chaenactis; Chaetadelpha; Chaetanthera; Chaetopappa; Chaetoseris; Chaetymenia; Chamaechaenactis; Chamaegeron; Chamaeleon; Chamaemelum; Chamaepeuce; Chamaepus; Chaptalia; Charadranaetes; Chardinia; Chartolepis; Cheirolophus; Cheliusia; Cherina; Chersodoma; Chevreulia; Chihuahuana; Childsia; Chiliadenus; Chiliocephalum; Chiliophyllum; Chiliotrichiopsis; Chiliotrichum; Chimantaea; Chionolaena; Chionopappus; Chlaenobolus; Chlamydophora; Chlamysperma; Chloracantha; Chondrilla; Chondropyxis; Chorisiva; Choristea; Chresta; Chromochiton; Chromolaena; Chromolepis; Chronopappus; ×Chrysaboltonia; Chrysactinia; Chrysactinium; Chrysanthellum; ×Chrysanthemoachillea; Chrysanthemoides; Chrysanthemum; Chrysanthoglossum; Chrysocephalum; Chrysocoma; Chrysocoryne; Chrysogonum; Chrysolaena; Chrysoma; Chrysophania; Chrysophthalmum; Chrysopsis; Chrysothamnus; Chthonia; Chthonocephalus; Chucoa; Chuquiraga; Cicerbita; Ciceronia; Cichorium; Cineraria; ×Cirsiocarduus; Cirsium; Cissampelopsis; Cladanthus; Cladochaeta; Cladopogon; Clappia; Clarionea; Clavigera; Clibadium; Clipteria; Cloiselia; Clomenocoma; Closia; Cnicothamnus; Cnicus; Codonocephalum; Coespeletia; Coleocoma; Coleosanthus; Coleostephus; Colobanthera; Cololobus; Columbiadoria; ×Colycea; ×Colymbacosta; Colymbada; Comaclinium; Comborhiza; Commidendrum; Condylidium; Condylopodium; Coniza; Conocliniopsis; Conoclinium; Constancea; ×Conygeron; Conyza; ×Conyzigeron; Coreocarpus; Coreopsis; Corethamnium; Corethrogyne; Coronidium; Corymbium; Cosmea; Cosmidium; Cosmophyllum; Cosmos; Cota; Cotula; Coulterella; Cousinia; Cousiniopsis; Craspedia; Crassocephalum; Cratystylis; Cremanthodium; Cremnothamnus; ×Crepi-Hieracium; Crepidiastrixeris; Crepidiastrum; Crepidifolium; Crepis; Crepula; Crinitaria; Criscia; Critonia; Critoniadelphus; Critoniella; Critoniopsis; Crocidium; Crocodilium; Crocodilodes; Cronquistia; Cronquistianthus; Croptilon; Crossolepis; Crossostephium; Crossothamnus; Crupina; Cryptopleura; Cryptostemma; Cuatrecasanthus; Cuatrecasasiella; Cuchumatanea; Culcitium; Cullumia; Cuniculotinus; Cupularia; Curio; Cuspidia; Cyanopis; Cyanopsis; Cyanthillium; Cyanus; Cyathocline; Cyathomone; Cyathopappus; Cyclolepis; Cylindrocline; Cymbolaena; Cymbonotus; Cymbopappus; Cymophora; Cynara; Cynaropsis; Cyrtocymura; Dacryotrichia; Dadia; Dahlia; Damnamenia; Damnxanthodium; Darwiniothamnus; Dasyandantha; Dasyanthina; Dasycondylus; Dasyphyllum; Dauresia; Daveaua; Decachaeta; Decaneuropsis; Decaneurum; Decastylocarpus; Decazesia; Deinandra; Delairea; Delamerea; Delilia; Delwiensia; Dendranthema; Dendrocacalia; Dendrophorbium; Dendrosenecio; Dendroseris; Dendroviguiera; Denekea; Denekia; Derderia; Desmanthodium; Detris; Dewildemania; Diacranthera; Diaperia; Diaphractanthus; Diaspananthus; Dicalymma; Dicercoclados; Dicerothamnus; Dichaetophora; Dichotoma; Dichrocephala; Dichromochlamys; Dicoma; Dicoria; Dicranocarpus; Dicrocephala; Didelta; Dielitzia; Dieteria; Digitacalia; Dilepis; Dillandia; Dimeresia; Dimerostemma; Dimorphocoma; Dimorphotheca; Dinoseris; Diodontium; Diplactis; Diplazoptilon; Diplemium; Diplopappus; Diplostephium; Dipterocome; Dipterocypsela; Disparago; Dissothrix; Distasis; Distegia; Distephanus; Disynaphia; Dithyrostegia; Ditrichum; Dittrichia; Doellia; Doellingeria; Dolichlasium; Dolichoglottis; Dolichogyne; Dolichorrhiza; Dolichothrix; Dolomiaea; Doniophyton; Doria; Dorobaea; Doronicum; Dresslerothamnus; Dubautia; Dubyaea; Dugaldia; Dugesia; Duhaldea; Duidaea; Dumerilia; Dusenia; Duseniella; Dymondia; Dyscritogyne; Dyscritothamnus; Dysodiopsis; Dyssodia; Eastwoodia; Eatonella; Echinacea; Echinocephalum; Echinocoryne; Echinops; Eclipta; Eclopes; Edmondia; Edwartiothamnus; Egania; Egletes; Eirmocephala; Eitenia; Eizaguirrea; Ekmania; Ekmaniopappus; Elachanthemum; Elachanthus; Elaphandra; Elekmania; Elephantopus; Elephantosis; Eleutheranthera; Ellenbergia; Elytropappus; Emilia; Emiliella; Enantiotrichum; Encelia; Enceliopsis; Endocellion; Endopappus; Endoptera; Engelmannia; Engleria; Enydra; Epallage; Epaltes; Epiclinastrum; Epilasia; Epitriche; Erato; Erechtites; Eremanthus; Eremosis; Eremothamnus; Eriachaenium; Ericameria; Ericentrodea; Erigeron; Eriocarpum; Eriocephalus; Eriochlamys; Eriocoryne; Eriolepis; Eriophyllum; Eriosphaera; Eriotrix; Erlangea; Erodiophyllum; Erymophyllum; Eryngiophyllum; Erythradenia; Erythrocephalum; Erythroseris; Eschenbachia; Espejoa; Espeletia; Espeletiopsis; Ethulia; Ethuliopsis; Eucephalus; Euchiton; Eumorphia; Eunoxis; Eupatoriastrum; Eupatorina; Eupatoriopsis; Eupatorium; Euphrosyne; Eurybia; Eurydochus; Euryops; Eutetras; Euthamia; Euthrixia; Eutrochium; Evacidium; Evax; Ewartia; Exomiocarpon; Faberia; Facelis; Farfugium; Farobaea; Faujasia; Faujasiopsis; Faxonia; Feddea; Feldstonia; Felicia; Fenixia; Ferreyranthus; Ferreyrella; Filaginella; Filaginopsis; Filago; ×Filfia; Filifolium; Fimbristima; Fingalia; Fitchia; Fitzwillia; Flaveria; Fleischmannia; Fleischmanniopsis; Florestina; Floscaldasia; Flosmutisia; Flotovia; Flourensia; Flyriella; Formania; Fornicium; Fougerouxia; Foveolina; Fragmosa; Franseria; Freemania; Freya; Frolovia; Fulcaldea; Gaillardia; Galactites; Galardia; Galatella; Galeana; Galeomma; Galinsoga; Gama; Gamochaeta; Gamochaetopsis; Gamolepis; Garberia; Garcibarrigoa; Garcilassa; Gardnerina; Garhadiolus; Garuleum; Gastrosulum; Gatyona; Gazania; Gazaniopsis; Geigeria; Geissolepis; Gelasia; Geraea; Gerbera; Geropogon; Gibbaria; Gifola; Gilberta; Gilruthia; Gladiopappus; Glebionis; Glossarion; Glossocardia; Glossogyne; Glossopappus; Glyphia; Glyptopleura; Gnaphaliothamnus; Gnaphalium; Gnephosis; Gnomophalium; Gochnatia; Goldmanella; Golionema; Gongrostylus; Gongrothamnus; Gongylolepis; Goniocaulon; Gonospermum; Gorceixia; Gorteria; Gossweilera; Goyazianthus; Grangea; Grangeopsis; Grantia; Graphistylis; Gratwickia; Grauanthus; Grazielia; Greenmaniella; Grindelia; Grisebachianthus; Grosvenoria; Guardiola; Guariruma; Guayania; Guayana; Guizotia; Gundelia; Gundlachia; Gutenbergia; Gutierrezia; Guynesomia; Gymnanthemum; Gymnarrhena; Gymnaster; Gymnocline; Gymnocondylus; Gymnocoronis; Gymnodiscus; Gymnolaena; Gymnolomia; Gymnopentzia; Gymnopsis; Gymnosperma; Gymnostephium; Gymnostyles; Gynema; Gynoxys; Gynura; Gypothamnium; Gyptidium; Gyptis; Gyrodoma; Haastia; Haeckeria; Haegiela; Haenelia; Hainanecio; Hamulium; Handelia; Hapalostephium; Haplocarpha; Haplodiscus; Haploesthes; Haplopappus; Haplostephium; Haptotrichion; Haradjania; Harleya; Harmonia; Harnackia; Harpaecarpus; Harpalium; Harpephora; Hartwrightia; Hasteola; Hatschbachiella; Havanella; Haxtonia; Hazardia; Hebeclinium; Hecastocleis; Hecatactis; Hectorea; Hedosyne; Hedypnois; Heleastrum; Helemonium; Helenia; Heleniastrum; Helenium; Helenomoium; Helepta; Heliantheae; Helianthella; Helianthopsis; Helianthus; Helichroa; Helichrysopsis; Helichrysum; Helicta; Heliocauta; Heliogenes; Heliomeris; Heliophthalmum; Heliopsis; Helioreos; Helipterum; Helminthia; Helminthotheca; Helogyne; Heloseris; Hemiambrosia; Hemilepis; Hemistepta; Hemisteptia; Hemixanthidium; Hemizonella; Hemizonia; Hemolepis; Henanthus; Henricksonia; Heptanthus; Heracantha; Heraclea; Herbichia; Herderia; Herodotia; Herreranthus; Herrickia; Hersilea; Hertia; Hesperevax; Hesperomannia; Hesperoseris; Heteracantha; Heteracia; Heteranthemis; Heterocoma; Heterocondylus; Heterocypsela; Heteroderis; ×Heterokalimeris; Heterolepis; Heteromera; Heteromma; Heteropappus; Heteropleura; Heteroplexis; Heterorachis; Heterorhachis; Heterosperma; Heterothalamulopsis; Heterothalamus; Heterotheca; Hidalgoa; Hierachium; Hieraciodes; Hieracioides; Hieracium; Hierapicra; Hilliardia; Hilliardiella; Himalaiella; Hingstonia; Hingtsha; Hinterhubera; Hiorthia; Hippia; Hippolytia; Hippophaestum; Himellia; Hirpicium; Hirtellina; Hispidella; Hoehnelia; Hoehnephytum; Hoffmanniella; Hofmeisteria; Hohenwartha; Holocarpha; Holocheilus; Hologymne; Hololeion; Hololepis; Holophyllum; Holoschkuhria; Holozonia; Homalotheca; Homogyne; Homoianthus; Homopappus; Homostylium; Hoorebekia; Hopkirkia; Hoplophyllum; Huarpea; Huberopappus; Hubertia; Huenefeldia; Hughesia; Hullsia; Hulsea; Hulteniella; Humbertacalia; Humea; Humeocline; Hutchinsonia; Hyalea; Hyalis; Hyalochaete; Hyalochlamys; Hyaloseris; Hyalosperma; Hybridella; Hydroidea; Hydropectis; Hylethale; Hymenatherum; Hymenocentron; Hymenocephalus; Hymenoclea; Hymenolepis; Hymenonema; Hymenopappus; Hymenostemma; Hymenostephium; Hymenothrix; Hymenoxys; Hyoseris; Hypacanthium; Hypericophyllum; Hypochaeris; Hypochoeris; Hysterionica; Hystrichophora; Ianthopappus; Ichthyothere; Ictinus; Idiopappus; Idiothamnus; Ifloga; Ignurbia; Iltisia; Imeria; Impia; Inezia; Infantea; Ingenhusia; Inkaliabum; Intybellia; Intybus; Inula; Inulanthera; Inulaster; Inuleae; Inuloides; Inulopsis; Inyonia; Io; Iocenes; Iodocephalopsis; Iodocephalus; Iogeton; Ionactis; Iostephane; Iotasperma; Iphiona; Iphionopsis; Iranecio; Irwinia; Ischnea; Ismelia; Isocarpha; Isocoma; Isoetopsis; Isomeria; Isostigma; Isotypus; Iteria; Iva; Ixauchenus; Ixeridium; Ixeris; Ixiochlamys; Ixiolaena; Ixodia; ×Ixyoungia; Jacea; Jaceacosta; Jaceitrapa; Jacmaia; Jacobaea; Jacobaeastrum; Jacobanthus; Jacosta; Jaegeria; Jalambica; Jalcophila; Jaliscoa; Jamesianthus; Jaramilloa; Jasonia; Jaumea; Jefea; Jeffreya; Jensia; Jessea; Joannea; Joannesia; Jobaphes; Johannia; Joseanthus; Jungia; Jurinea; Jurinella; Kaulfussia; Kalimares; Kalimeris; Kallias; Kanimia; Karelinia; Karvandarina; Kaschgaria; Kaulfussia; Kaunia; Keerlia; Kemulariella; Kentrophyllum; Keringa; Kerneria; Keysseria; Khasianthus; Kiliana; Kinghamia; Kingianthus; Kippistia; Klasea; Klaseopsis; Kleinia; Klenzea; Koanophyllon; Koechlea; Koehneola; Koelpinia; Kovalevskiella; Koyamacalia; Koyamasia; Krigia; Krylovia; Kuhnia; Kyhosia; Kymapleura; Kyrstenia; Kyrsteniopsis; Lachanodes; Lachnophyllum; Lachnorhiza; Lachnospermum; Lachnothalamus; Lacinaria; Lactuca; Lactucopsis; Laennecia; Laestadia; Lagascea; Lagenocypsela; Lagenopappus; Lagenophora; Laggera; Lagophylla; Lagoseriopsis; Lagoseris; Lagothamnus; Lagurostemon; Lalda; Lamprachaenium; Lamprocephalus; Lampropappus; Lampsana; Lamyra; Lamyropappus; Lamyropsis; Lancisia; Landtia; Langebergia; Lanipila; Lantanopsis; Laphamia; Laphangium; Lappa; Lapsana; Lapsanastrum; Lapsyoungia; Lasallea; Lasianthaea; Lasiocarphus; Lasiocephalus; Lasiolaena; Lasiopogon; Lasiorrhiza; Lasiospermum; Lasiospora; Lasthenia; Latreillea; Launaea; Launaya; Launea; Lavenia; Lawrencella; Laxanon; Laxmannia; Laxopetalum; Layia; Leachia; Lebetina; Lecocarpus; Leiachenis; Leibnitzia; Leiboldia; Leighia; Leiocarpa; Leiodon; Leioligo; Lemmatium; Lemooria; Leonis; Leontodon; Leontonyx; Leontopodium; Lepachis; Lepachys; Lepicaune; Lepidaploa; Lepidesmia; Lepidolopha; Lepidolopsis; Lepidonia; Lepidopappus; Lepidophorum; Lepidophyllum; Lepidopogon; Lepidoseris; Lepidospartum; Lepidostephanus; Lepidostephium; Lepidotheca; Lepiscline; Leptalea; Lepteranthus; Leptica; Leptilon; Leptinella; Leptocarpha; Leptoclinium; Leptogyne; Leptopoda; Leptorhynchos; Leptoseris; Leptostelma; Leptosyne; Leptotis; Leptotriche; Leria; Lescaillea; Lessingia; Lessingianthus; Leto; Leucacantha; Leucactinia; ×Leucantanacetum; Leucantha; Leucanthemella; Leucanthemopsis; Leucanthemum; Leucelene; Leuchaeria; Leucheria; Leuciva; Leucoblepharis; Leucochrysum; Leucogenes; Leucomeris; Leucopholis; Leucophyta; Leucopsidium; Leucopsis; Leucoptera; Leucoseris; Leunisia; Leuzea; Leveillea; Leysera; Liabellum; Liabum; Liatris; Libanothamnus; Lidbeckia; Lieberkuhna; Lifago; Ligularia; Ligulariopsis; Limbarda; Lindheimera; Linochilus; Linosyris; Linsecomia; Linzia; Lipochaeta; Lipotriche; Lipschitziella; Lipskyella; Litogyne; Litothamnus; Litrisa; Llerasia; Logfia; Lomanthus; Lomatozona; Lomaxeta; Lonas; Lophactis; Lophiolepis; Lophoclinium; Lopholaena; Lopholoma; Lophopappus; Lorandersonia; Lordhowea; Lorentea; Lorentzianthus; Loricaria; Lourteigia; Loxodon; Loxothysanus; Lucilia; Luciliocline; Luina; Lulia; Lundellianthus; Lundinia; Lupsia; Lycapsus; Lychnocephaliopsis; Lychnocephalus; Lychnophora; Lychnophoriopsis; Lycoseris; Lycotis; Lygodesmia; Lyonnetia; Lysistemma; Machaeranthera; Machlis; Macledium; Macowania; Macrachaenium; Macraea; Macroclinidium; Macronema; Macropertya; Macropodina; Macvaughiella; Madagaster; Madaractis; Madaria; Madaroglossa; Madea; Madia; Mairia; Malacocephalus; Malacothrix; Malmeanthus; Malperia; Mandonia; Mantagnaea; Mantisalca; Manyonia; Marasmodes; Marcelia; Margarita; Mariacantha; Mariana; Marizia; Marsea; Marshallia; Marshalljohnstonia; Marticorenia; Martrasia; Maruta; Mastrucium; Matricaria; Mattfeldanthus; Mattfeldia; Matudina; Mauranthemum; Mausolea; Mazzettia; Mecomischus; Medicusia; Medranoa; Megalodonta; Melampodium; Melananthera; Melanchrysum; Melanodendron; Melanoloma; Melanoseris; Melanthera; Melarhiza; Melissopsis; Menomphalus; Meratia; Merrittia; Mesadenia; Mesanthophora; Mesocentron; Mesodetra; Mesogramma; Mesoneuris; Metabasis; Metagnanthus; Metalasia; Metastevia; Meteorina; Mexerion; Mexianthus; Meyerafra; Meyeria; Micractis; Micrauchenia; Micrelium; Microbahia; Microcephala; Microcephalum; Microchaeta; Microchaete; Microcoecia; Microderis; Microglossa; Microgyne; Microlecane; Microliabum; Microlonchus; Microlophopsis; Microlophus; Micropsis; Micropus; Microrhynchus; Microseris; Microspermum; Mikania; Mikaniopsis; Millefolium; Miliaria; Millina; Millotia; Minasia; Minuria; Minythodes; Miradoria; Mirasolia; Miricacalia; Misbrookea; Mitina; Miyamayomena; Mnesiteon; Mniodes; Mocinia; Moerkensteinia; Molpadia; Monactis; Monarrhenus; Monencyanthes; Monenteles; Monoculus; Monogereion; Monolopia; Monopholis; Monoptilon; Monosis; Monothrix; Montagnaea; Montanoa; Monticalia; Moonia; Moquinia; Morithamnus; Morna; Morysia; Moscharia; Moschifera; Mosigia; Msuata; Mtonia; Mulgedium; Munnozia; Munzothamnus; Muschleria; Musilia; Mussinia; Musteron; Mutisia; Myanmaria; Myconia; Myctanthes; Myopordon; Myriactis; Myriocephalus; Myripnois; Myscolus; Myxopappus; Nabalus; Nablonium; Nacrea; Nananthea; Nannoglottis; Nanothamnus; Narbalia; Nardophyllum; Nardosmia; Narvalina; Nassauvia; Nauenburgia; Nauplius; Neactelis; Neblinaea; Neesia; Neja; Nemolepis; Nemosenecio; Neo-taraxacum; Neocabreria; Neoceis; Neocuatrecasia; Neohintonia; Neojeffreya; Neomirandea; Neomolina; Neonesomia; Neopallasia; Neosyris; Neotysonia; Nephrotheca; Nesampelos; Nesomia; Nestlera; Nestotus; Neurelmis; Neurolaena; Neurolakis; Nicolasia; Nicolletia; Nidorella; Nikitinia; Nipponanthemum; Nitelium; Nivellea; Nocca; Nolletia; Nordenstamia; Norlindhia; Nothobaccharis; Nothocalais; Noticastrum; Notobasis; Notonia; Notoseris; Nouelia; Novaguinea; Novenia; Novopokrovskia; Oaxacania; Obaejaca; Obefiscaria; Oblivia; Ochrocephala; Ochronelis; Oclemena; Ocneron; Odixia; Odoglossa; Odontocline; Odontolophus; Odontoptera; Odontospermum; Odontotrichum; Oedera; Oegroe; Ogiera; Oglifa; Oiospermum; Oldenburgia; Oldfeltia; Olearia; Olgaea; Ofigactis; Oligandra; Oliganthemum; Oliganthes; Oligocarpus; Ofigochaeta; Oligoglossa; Oligogyne; Oligolepis; Oligoneuron; Oligosporus; Oligothrix; Olivaea; Omalanthus; Omalocline; Omalotes; Omalotheca; Omphalopappus; Oncosiphon; Ondetia; Onobroma; Onopix; Onopordum; Onopyxus; Onoseris; Onotrophe; Oocephala; Ooclinium; Oonopsis; Oparanthus; Ophryosporus; Opicrina; Opisthopappus; Oporinia; Orbivestus; Oreochrysum; Oreoleysera; Oreophila; Oreoseris; Oreostemma; Oresbia; Oresigonia; Oriastrum; Oritrophium; Ormenis; Orochaenactis; Orsina; Orthocentron; Orthopappus; Osbertia; Osmadenia; Osmia; Osmiopsis; Osmites; Osmitiphyllum; Osmitopsis; Osteospermum; Oswalda; Otanthus; Oteiza; Othake; Othonna; Othonnopsis; Otochlamys; Otopappus; Otospermum; Oxiphoeria; Oxycarpha; Oxylaena; Oxylobus; Oxypappus; Oxyphyllum; Oxytenia; Oxyura; Oyedaea; Ozothamnus; Pachyderis; Pachylaena; Pachystegia; Pachythamnus; Pacifigeron; Packera; Pacourina; Paenula; Palafoxia; Paleaepappus; Paleista; Paleolaria; Paleya; Pallenis; Pamphalea; Panaetia; Panargyrus; Paneroa; Paniopsis; Panphalea; Pappobolus; Pappochroma; Papuacalia; Paquerina; Paracalia; Parachionolaena; Parafaujasia; Paragynoxys; Paralychnophora; Paramiflos; Paranephelius; Parantennaria; Paraphysis; Parapiqueria; Parapolydora; Paraprenanthes; Parasenecio; Parastrephia; Parasyncalathium; Pardisium; Parthenice; Parthenium; Parthenopsis; Pasaccardoa; Pascalia; Paurolepis; Pechuel-Ioeschea; Pectinastrum; Pectis; Pegolettia; Peltidium; Pelucha; Pembertonia; Pentacalia; Pentachaeta; Pentalepis; Pentanema; Pentaphorus; Pentataxis; Pentatrichia; Pentzia; Peramibus; Perdicium; Pereuphora; Perezia; Pericalia; Pericallis; Pericome; Peripleura; Peritris; Perkyle; Perplexia; Perralderia; Perralderiopsis; Personaria; Pertya; Perymeniopsis; Perymenium; Petalacte; Petalolepis; Petasites; Peteravenia; Petradoria; Petrobium; Peucephyllum; Phacellothrix; Phaenixopus; Phaenocoma; Phaeopappus; Phaethusa; Phagnalon; Phalacrachena; Phalacraea; Phalacrocarpum; Phalacrodiscus; Phalacroloma; Phalacromesus; Phalacroseris; Phalolepis; Phaneroglossa; Phanerostylis; Phania; Phialis; Philactis; Phileozera; Philoglossa; Philostizus; Philyrophyllum; Phitosia; Phoebanthus; Phonus; Phrygia; Phyllimena; Phyllocephalum; Phyllostefidium; Phymaspermum; Phyteumopsis; Picnomon; Picradenia; Picradeniopsis; Picridium; Picris; Picrosia; Picrothamnus; Pilosella; Pilostemon; Pinardia; Pinaropappus; Pingraea; Pinillosia: Piora; Pippenalia; Piptocarpha; Piptocephalum; Piptoceras; Piptocoma; Piptolepis; Piptopogon; Piptothrix; Piqueria; Piqueriella; Piqueriopsis; Pirarda; Pithecoseris; Pithocarpa; Pittocaulon; Pityopsis; Placus; Pladaroxylon; Plagiobasis; Plagiochellus; Plagiolophus; Plagius; Planaltoa; Planea; Plateilema; Platycarpha; Platychaete; Platycheilus; Platypodanthera; Platyraphium; Platyschkuhria; Platzchaeta; Plazia; Plecostachys; Plectocephalus; Pleiacanthus; Pleiogyne; Pleiotaxis; Pleocarphus; Pleurocarpaea; Pleurocoronis; Pleuropappus; Pleurophyllium; Pluchea; Plumosipappus; Podachaenium; Podanthus; Podocoma; Podolepis; Podosperma; Podospermum; Podotheca; Poecilolepis; Poecilotriche; Pogonolepis; Pojarkovia; Poljakanthema; Poljakovia; Pollalesta; Poloa; Polyacantha; Polyachyrus; Polyactidium; Polyactis; Polyanthina; Polyarrhena; Polycalymma; Polycantha; Polychaetia; Polychrysum; Polydora; Polymnia; Polymniastrum; Polypappus; Polypteris; Polytaxis; Pontesia; Pontia; Porcellites; Porophyllum; Porphyrostemma; Portalesia; Postia; Praxefiopsis; Praxefis; Prenanthella; ×Prenanthenia; Prenanthes; Prestelia; Prestinaria; Printzia; Prolobus; Prolongoa; Pronacron; Proteopsis; Proustia; Psacaliopsis; Psacalium; Psammoseris; Psanacetum; Psathyrotes; Psathyrotopsis; Psectra; Psednotrichia; Psephellus; Pseudelephantopus; Pseudobaccharis; Pseudobahia; Pseudoblepharispermum; Pseudobrickellia; Pseudoclappia; Pseudoglossanthis; Pseudognaphalium; Pseudogynoxys; Pseudohandelia; Pseudojacobaea; Pseudokyrsteniopsis; Pseudoligandra; Pseudolinosyris; Pseudonoseris; Pseudopiptocarpha; Pseudostifftia; Pseudoyoungia; Psiadia; Psiadiella; Psila; Psilactis; Psilocarphus; Psilostrophe; Psora; Psychrogeton; Ptarmica; Pterachaenia; Pterigeron; Pternix; Pterocaulon; Pterochaeta; Pterocladis; Pterocypsela; Pterolophus; Pteronia; Pterophorus; Pterophyton; Pteropogon; Pterosenecio; Pterostephanus; Pterotheca; Pterothrix; Pterygopappus; Ptilepida; Ptileris; Ptilomeris; Ptilonella; Ptiloria; Ptilosia; Ptilostemon; Ptilostephium; Ptosimopappus; Pugiopappus; Pulicaria; Punduana; Pycnocomus; Pycnosorus; Pyrethraria; Pyrethropsis; Pyrethrum; Pyropsis; Pyrrhopappus; Pyrrocoma; Pytinicarpa; Quechualia; Quelchia; Quinetia; Quinqueremulus; Rachelia; Radlkoferotoma; Rafinesquia; Raillardella; Raillardia; Railliardia; Rainiera; Rancagua; Raoulia; Raouliopsis; Rastrophyllum; Ratibida; Raulinoreitzia; Rayjacksonia; Reichardia; Relhania; Remya; Rennera; Rensonia; Resinocaulon; Revealia; Rhabdotheca; Rhacoma; Rhagadiolus; Rhamphogyne; Rhanteriopsis; Rhanterium; Rhapontica; Rhaponticoides; Rhaponticum; Rhetinocarpha; Rhetinodendron; Rhetinolepis; Rhinactina; Rhinactinidia; Rhodanthe; Rhodanthemum; Rhodogeron; Rhynchocarpus; Rhynchopappus; Rhynchopsidium; Rhynchospermum; Rhysolepis; Richardia; Richterago; Richteria; Ridan; Ridania; Riddellia; Riencourtia; Rigiopappus; Robinsonecio; Robinsonia; Roccardia; Rochonia; Rodigia; Rohria; Rojasianthe; Rolandra; Roldana; Roodebergia; Rosenia; Rothmaleria; Rudbeckia; Rugelia; Ruilopezia; Rumfordia; Russowia; Rutidosis; Rydbergia; Sabazia; Sabbata; Sachsia; Sagmen; Saintmorysia; Salcedoa; Saimaa; Salmeopsis; Santolina; Santonica; Santosia; Sanvitalia; Sarcanthemum; Sartorina; Sartwellia; Saubinetia; Saussurea; Saussuria; Scabrethia; Scalesia; Scalia; Scaliopsis; Scariola; Scepinia; Schaetzellia; Scherya; Scheuchleria; Schischkinia; Schistocarpha; Schistostephium; Schizogyne; Schizoptera; Schizotrichia; Schkuhria; Schlagintweitia; Schlechtendalia; Schmalhausenia; Schmidtia; Schoenia; Schortia; Schumeria; Sciadioseris; Sciadocephala; Sclerobasis; Sclerocarpus; Sclerolepis; Sclerorhachis; Scolospermum; Scolymanthus; Scolymus; Scorzonella; Scorzonera; Scorzoneroides; Scrobicaria; Scyphocoronis; Scyphopappus; Scytala; Sebastiania; Selleophytum; Selloa; Semiria; Senecillicacalia; Seneciffis; Senecio; Senecioneae; Seneciunculus; Sericocarpus; Seridia; Seriola; Seriphidium; Seriphium; Seris; Serpaea; Serratula; Seruneum; Setachna; Shafera; Shawia; Sheareria; Shinnersia; Shinnersoseris; Siapaea; Sideranthus; Siebera; Siemssenia; Sigesbeckia; Siloxerus; Silphion; Silphium; Silybum; Simlera; Simsia; Sinacalia; Sinclairia; Sinoleontopodium; Sinosenecio; Sipolisia; Skirrhophorus; Smallanthus; Soaresia; Sobreyra; Sogalgina; Solanecio; Soldevilla; Solenogyne; Solenotheca; Solidago; ×Solidaster; Sofiva; Solstitiaria; Sommerfeltia; Sonchella; Sonchoseris; Sonchus; Sonchustenia; Sondottia; Soroseris; Soyeria; Spadactis; Spadonia; Spaniopappus; Spanotrichum; Sparganophorus; Spathipappus; Sphacophyllum; Sphaeranthus; Sphaereupatorium; Sphaeromeria; Sphaeromorphaea; Sphagneticola; Sphenogyne; Spilacron; Spilanthes; Spiracantha; Spiralepis; Spiropodium; Spiroseris; Spitzelia; Spongotrichum; Sprunira; Sprunnera; Squamopappus; Stachycephalum; Staebe; Staehelina; Stammarium; Standleyanthus; Stanfieldia; Staurochlamys; Stechmannia; Stegonotus; Steiractinia; Steirodiscus; Steiroglossa; Stemmacantha; Stemmatella; Stemmodontia; Stenachaenium; Stenactis; Stenocarpha; Stenocephalum; Stenocline; Stenopadus; Stenophalium; Stenophyllum; Stenops; Stenoseris; Stenotheca; Stenotus; Stephanbeckia; Stephanochilus; Stephanocoma; Stephanodoria; Stephanomeria; Stephanopappus; Stephanopholis; Steptorhamphus; Stera; Stereosanthus; Steriphe; Stevia; Steviopsis; Steyermarkina; Sthaelina; Stifftia; Stigmatotheca; Stilpnogyne; Stilpnolepis; Stilpnopappus; Stizolophus; Stobaea; Stoebe; Stokesia; Stomatanthes; Stomatochaeta; Stramentopappus; Streckera; Streptoglossa; Strobocalyx; Strophopappus; Strotheria; Struchium; Stuartina; Stuckertiella; Stuessya; Stylimnus; Stylocline; Stylolepis; Styloncerus; Stylopappus; Stylotrichium; Succisocrepis; Swammerdamia; Symphipappus; Symphyllocarpus; Symphyochaeta; Symphyopappus; Symphyotrichum; Syncalathium; Syncarpha; Syncephalum; Synchaeta; Synchodendron; Syncretocarpus; Synedrella; Synedrellopsis; Syneilesis; Synosma; Synotis; Syntrichopappus; Synurus; Syreitschikovia; Tafalla; Tagetes; Takhtajaniantha; Talamancalia; Tamananthus; Tamania; Tamaulipa; Tanacetopsis; Tanacetum; Tanaxion; Taplinia; Taraxacum; Tarchonanthus; Tarlmounia; Tehuana; Teichostemma; Teixeiranthus; Telanthophora; Telekia; Telesia; Telmatophila; Tenrhynea; Tephroseris; Tepion; Terana; Tessaria; Tessenia; Tetracanthus; Tetracarpum; Tetrachyron; Tetradymia; Tetragonosperma; Tetragonotheca; Tetramolopium; Tetraneuris; Tetrantha; Tetranthus; Tetraotis; Tetraperone; Tetrodus; Thaminophyllum; Thamnoseris; Thelesperma; Therogeron; Therolepta; Thespidium; Thespis; Thevenotia; Thinobia; Thiseltonia; Thorelia; Thrincia; Thrixia; Thurovia; Thymophylla; Thymopsis; Thyopsis; Thyrsanthema; Tiarocarpus; Tibetoseris; Tietkensia; Tilesia; Tithonia; Toiyabea; Tolbonia; Tollatia; Tolpis; Tomanthea; Tomentaurum; Tonestus; Torrentia; Tostimontia; Tourneuxia; Townsendia; Toxanthes; Trachodes; Tracyina; Tragopogon; Tragopogonoides; Trallesia; Trattenikia; Traversia; Trepadonia; Triachne; Trichaetolepis; Trichanthemis; Trichanthodium; Trichocline; Trichocoronis; Trichocoryne; Trichocrepis; Trichogonia; Trichogoniopsis; Trichogyne; Tricholepis; Trichoptilium; Trichoseris; Trichospira; Trichostemma; Trichostephium; Trichymenia; Tridactylina; Tridax; Trigonopterum; Trigonospermum; Trilisa; Trimeranthes; Trimetra; Trimorpha; Triniteurybia; Trioncinia; xTripleurocota; Tripleurospermum; ×Tripleurothemis; Triplocentron; Triplocephalum; Triplotaxis; Tripolion; Tripolium; Tripteris; Triptilion; Triptilium; Triptilodiscus; Trixis; Trochoseris; Troglophyton; Tropidolepis; Troximon; Tuberculocarpus; Tuberostylis; Tubilium; Tuckermannia; Tugarinovia; Tulakenia; Tumionella; Turaniphytum; Turczaninowia; Tursenia; Tussilago; Tuxtla; Tyleropappus; Tylloma; Tyrimnus; Tzvelevopyrethrum; Ubiaea; Uechtritzia; Ugamia; Uhdea; Uleophytum; Ulina; Unamia; Unxia; Urbananthus; Urbanisol; Urbinella; Urmenetea; Urolepis; Uropappus; Urospermum; Urostylis; Ursinia; Vanillosma; Vanillosmopsis; Vargasia; Varilla; Varthemia; Vasquezia; Vellereophyton; Venatris; Vendredia; Venegasia; Venegazia; Venidium; Verbesina; Vernasolis; Vernonanthura; Vemonella; Vernonia; Vernoniastrum; Vernonieae; Vernoniopsis; Verutina; Vicoa; Vieraea; Viereckia; Vierhapperia; Vigethia; Vigolina; Viguiera; Villanova; Villasenoria; Vinicia; Virgaria; Virginea; Virgulaster; Virgulus; Vittadinia; Vittetia; Vladimiria; Volutarella; Volutaria; Wahlenbergia; Waitzia; Waldheimia; Wamalchitamia; Wardaster; Warionia; Wedelia; Welwitschiella; Werneria; Westoniella; Wettsteinia; Wiborgia; Wiestia; Wilkesia; Willemetia; Willoughbya; Willugbaeya; Wollastonia; Woodvillea; Wootonia; Wuerschmittia; Wulffia; Wunderlichia; Wyethia; Wyomingia; Xalkitis; Xanthidium; Xanthisma; Xanthium; Xantho; Xanthocephalum; Xanthochrysum; Xanthocoma; Xantholepis; Xanthopappus; Xanthophthalmum; Xanthopsis; Xenocarpus; Xenophontia; Xenophyllum; Xeranthemum; Xerobius; Xerochrysum; Xerolekia; Xeroloma; Xeropappus; Xerotium; Xerxes; Xetoligus; Ximenesia; Xiphochaeta; Xylanthemum; Xylorhiza; Xylothamia; Xylovirgata; Yermo; Youngia; Yunquea; Zacintha; Zaluzania; Zarabellia; Zemisia; Zexmenia; Zinnia; Zoegea; Zollikoferia; Zoutpansbergia; Zyrphelis; Zyzyxia.

FAMILY: COMPOSITAE; GENERA: HELIANTHUS; SPECIES: Helianthus agrestis Pollard—southeastern sunflower; Helianthus ambiguus Britt.—Ambiguous Sunflower; Helianthus angustifolius L.—swamp sunflower; Helianthus annuus L.—common sunflower, girasol (Spanish); Helianthus anomalus S. F. Blake—western sunflower; Helianthus argophyllus Torr. & A. Gray—silverleaf sunflower; Helianthus arizonensis R. C. Jacks.—Arizona sunflower; Helianthus atrorubens L.—purpledisk sunflower; Helianthus bolanderi A. Gray—serpentine sunflower; Helianthus×brevifolius E. Watson—shortleaf sunflower; Helianthus califomicus DC.—California sunflower; Helianthus carnosus Small—lakeside sunflower; Helianthus ciliaris DC.—Texas blueweed; Helianthus cinereus Small; Helianthus coloradensis Cockerell—prairie sunflower; Helianthus cusickii A. Gray—Cusick's sunflower; Helianthus debilis Nutt.—cucumberleaf Sunflower; Helianthus decapetalus L.—thinleaf sunflower; Helianthus deserticola Heiser—desert sunflower; †Helianthus diffusus Sims; Helianthus dissectifolius R. C. Jacks; Helianthus divaricatus L.—woodland sunflower or rough woodland sunflower; Helianthus×divariserratus R. W. Long; Helianthus×doronicoides Lam.; Helianthus exilis A. Gray; Helianthus floridanus A. Gray ex Chapm.—Florida sunflower; Helianthus giganteus L.—giant sunflower; Helianthus glaucophyllus D. M. Sm—whiteleaf sunflower; Helianthus×glaucus Small; Helianthus gracilentus A. Gray—slender sunflower; Helianthus grosseserratus M. Martens—sawtooth sunflower; Helianthus heterophyllus Nutt.—variableleaf sunflower; Helianthus hirsutus Raf.—hairy sunflower; Helianthus×intermedius R. W. Long—intermediate sunflower; Helianthus Iaciniatus A. Gray—alkali sunflower; Helianthus×Iaetiflorus Pers.—cheerful sunflower, mountain sunflower; Helianthus laevigatus Torr. & A. Gray—smooth sunflower; Helianthus lenticularis Douglas ex Lindl.; Helianthus longifolius Pursh—longleaf sunflower; Helianthus×Iuxurians (E. Watson) E. Watson; Helianthus maximiliani Schrad.—Maximillian sunflower; Helianthus membranifolius Poir.; Helianthus mollis Lam.—downy sunflower, ashy sunflower; Helianthus multiflorus L.—manyflower sunflower; Helianthus navarri Phil.; Helianthus neglectus Heiser—neglected sunflower; Helianthus niveus (Benth.) Brandegee—showy sunflower; Helianthus nuttallii Torr. & A. Gray; Helianthus occidentalis Riddell—fewleaf sunflower, western sunflower; Helianthus×orgyaloides Cockerell; Helianthus paradoxus Heiser—paradox sunflower; Helianthus pauciflorus Nutt.—stiff sunflower; Helianthus petiolaris Nutt.—prairie sunflower, lesser sunflower; Helianthus porteri (A. Gray) Pruski—Porter's sunflower; Helianthus praecox Engelm. & A. Gray Texas sunflower; †Helianthus praetermissus—New Mexico sunflower; Helianthus pumilus Nutt.—little sunflower; Helianthus radula (Pursh) Torr. & A.Gray—rayless sunflower; Helianthus resinosus Small—rescindot sunflower Helianthus salicifolius A. Dietr.—willowleaf sunflower; Helianthus sarmentosus Rich.—French Guiana; Helianthus scaberrimus Elliott; Helianthus schweinitzii Torr. & A. Gray—Schweinitz's sunflower; Helianthus silphioides Nutt.—rosinweed sunflower; Helianthus simulans E. Watson—muck sunflower; Helianthus smithii Heiser—Smith's sunflower; Helianthus speciosus Hook.—Michoacan; Helianthus subcanescens (A. Gray) E. Watson; Helianthus subtuberosus Bourg.; Helianthus tuberosus L.—Jerusalem artichoke, sunchoke, earth-apple, topinambur; Helianthus×verticillatus Small—whorled sunflower.

FAMILY: ASTERACEAE; GENERA: Aaronsohnia Warb. & Eig; Abrotanella Cass.; Acamptopappus (A. Gray) A. Gray—goldenhead; Acanthocephalus Kar. & Kir.; Acanthocladium F. Muell.; Acanthodesmos C. D. Adams & duQuesnay; Acantholepis Less.; Acanthospermum Schrank—starburr; Acanthostyles R. M. King & H. Rob.; Achaetogeron A. Gray; Achillea L.—yarrow; Achnophora F. Muell.; Achnopogon Maguire, Steyerm. & Wurdack; Achyrachaena Schauer—blow wives; Achyrocline (Less.) DC.; Achyropappus Kunth; Achyrothalamus O. Hoffm.; Acmella Rich.; Acomis F. Muell.; Acourtia D. Don—desert peony; Acrisione B. Nord.; Acritopappus R. M. King & H. Rob.; Acroclinium A. Gray; Acroptilon Cass—hardheads, Russian knapweed; Actinobole Endl.; Actinoseris (Endl.) Cabrera; Actinospermum Elliott; Adelostigma Steetz; Adenanthellum B. Nord.; Adenocaulon Hook—trailplant; Adenocritonia R. M. King & H. Rob.; Adenoglossa B. Nord.; Adenoon Dalzell; Adenopappus Benth; Adenophyllum—dogweed; Adenostemma Pers.—medicineplant; Adenostyles A. Kern.; Adenothamnus D. D.Keck; Aedesia O.Hoffm.; Aegopordon Boiss.; Aequatorium B. Nord; Aetheorhiza Cass.; Ageratella A. Gray ex S.Watson; Ageratina Spach—snakeroot; Ageratinastrum Mattf.; Ageratum L.—whiteweed; Agiabampoa Rose ex O. Hoffm.; Agnorhiza (Jeps.) W. A. Weber; Agoseris; Raf.—mountain dandelion; Agrianthus Mart. ex DC.; Ainsliaea DC.; Ajania Poljakov; Ajaniopsis C. Shih; Alatoseta Compton; Albertinia Spreng.; Alcantara Glaz. ex G. M. Barroso; Alciope DC. ex Lindl.; Aldama La Llave; Alepidocline S. F. Blake; Alfredia Cass.; Aliella Qaiser & Lack; Allagopappus Cass.; Allardia Decne.; Alloispermum Willd.; Allopterigeron Dunlop; Almutaster—alkali marsh aster (synonym of Aster L.); Alomia Kunth; Alomiella R. M. King & H. Rob.; Alvordia Brandegee; Amauria Benth; Amberboa (Pers.) Less.; Amblyocarpum Fisch. & C. A. Mey.; Amblyolepis DC; Amblyopappus Hook. & Am.; Amboroa Cabrera; Ambrosia L.—bursage, ragweed; Ameghinoa Speg.; Amellus L.; Ammobium R. Br. ex Sims; Amolinia R. M. King & H. Rob.; Ampelaster—climbing aster; Amphiachyris—broomweed; Amphiglossa DC; Amphipappus—chaffbush; Amphoricarpos Vis.; Anacantha (Iljin) Sojak; Anacyclus L.; Anaphalioides (Benth.) Kirp.; Anaphalis DC—pearly everlasting; Anastraphia D. Don; Anaxeton Gaertn.; Ancathia DC.; Ancistrocarphus; Ancistrophora A. Gray; Anderbergia; Andryala L.; Anemocarpa; Angelphytum G.M. Barroso; Angianthus J. C. Wendl.; Anisochaeta DC.; Anisocoma Torr. & A. Gray; Anisopappus Hook. & Arn.; Anisothrix O. Hoffm. ex Kuntze; Anomostephium DC.; Antennaria Gaertn.—pussytoes; Anthemis L.—Roman chamomile; Antheropeas Rydb.—Easter bonnets; Antillia R. M. King & H. Rob.; Antiphiona Merxm.; Antithrixia DC.; Anura (Juz.) Tscherneva; Anvillea DC.; Apalochlamys (Cass.) Cass.; Apargidium Torr. & A. Gray; Aphanactis Wedd.; Aphanostephus DC—doze daisy; Aphyllocladus Wedd.; Apodocephala Baker; Aposeris Neck. ex Cass.; Apostates Lander; Arbelaezaster Cuatrec.; Archibaccharis Heering; Arctanthemum (Tzvelev) Tzvelev; Arctium L.—burdock; Arctogeron DC.; Arctotheca J. C. Wendl.—capeweed; Arctotis L.; Argentipallium Paul G. Wilson; Argyranthemum Webb—dill daisy; Argyroglottis Turcz.; Argyrophanes Schltdl.; Argyroxiphium DC—silversword; Arida; Aristeguietia R.M. King & H. Rob.; Amaldoa Cabrera; Arnica L.—arnica; Arnicastrum Greenm.; Arnoglossum Raf.—Indian plantain; Arnoseris Gaertn.; Arrhenechthites Mattf.; Arrojadocharis Mattf.; Arrowsmithia DC.; Artemisia L.—tarragon, sagebrush, sagewort, wormwood, mugwort; Artemisiopsis S. Moore; Asanthus R. M. King & H. Rob.—brickellbush; Ascidiogyne Cuatrec; Aspilia Thouars; Asplundianthus R. M. King & H. Rob; Aster L.—aster; Asteridea Lindl.; Asteriscus Mill.; Asteromoea Blume; Astranthium Nutt.—western daisy; Athanasia L.; Athrixia Ker Gawl.; Athroisma DC; Atractylis L.; Atractylodes DC.; Atrichantha Hilliard & B. L. Burtt; Atrichoseris A. Gray; Austrobrickellia R. M. King & H. Rob.; Austrocritonia R. M. King & H. Rob.; Austroeupatorium R. M. King & H. Rob.; Austrosynotis C. Jeffrey; Avellara Blanca & C.Diaz; Axiniphyllum Benth; Ayapana Spach; Ayapanopsis R. M. King & H. Rob.; Aylacophora Cabrera; Baccharis L.—baccharis; Badilloa R. M. King & H. Rob.; Baeriopsis J. T. Howell; Bafutia C. D. Adams; Bahia Lach.—bahia; Bahianthus R. M. King & H. Rob.; Baileya Harv. & A. Gray—desert marigold; Bajacalia; Balduina Nutt.—honeycombhead; Balsamorhiza Hook. ex Nutt.—balsamroot; Baltimora L.—baltimora; Barkleyanthus H. Rob. & Brettell—willow ragwort; Barnadesia Mutis ex L.f.; Barroetea A. Gray; Barrosoa R. M. King & H. Rob.; Bartlettia A. Gray; Bartlettina R. M. King & H. Rob.; Basedowia E.Pritz.; Bebbia Greene—sweetbush; Bedfordia DC.; Bejaranoa R. M. King & H. Rob.; Bellida Ewart; Bellis L.—daisy; Bellium L.; Belloa J. Remy; Benitoa D. D.Keck; Berardia Vill.; Berkheya Ehrh.; Berlandiera DC—greeneyes; Berroa Beauverd; Bethencourtia; Bidens L.—beggartick, devil's sticktight, Spanish needles; Bigelowia DC—rayless goldenrod; Bishopanthus H. Rob.; Bishopiella R. M. King & H. Rob.; Bishovia R. M. King & H. Rob.; Blainvillea Cass.; Blakeanthus R. M. King & H. Rob.; Blakiella Cuatrec.; Blanchetia DC; Blennosperma Less.—stickyseed; Blennospora A. Gray; Blepharipappus Hook; Blepharispermum DC.; Blepharizonia (A. Gray) Greene; Blumea DC.—false oxtongue; Blumeopsis Gagnep.; Boeberastrum (A. Gray) Rydb.; Boeberoides (DC.) Strother; Boltonia L′Her.—doll's daisy; Bombycilaena (DC.) Smoljan.; Borrichia Adans.—seaside tansy; Bothriocline Oliv. ex Benth.; Brachanthemum DC.; Brachionostylum Mattf.; Brachyactis—rayless aster; Brachyglottis J. R. Forst. & G. Forst.; Brachylaena R. Br.; Brachyscome Cass.; Brachythrix Wild & G. V. Pope; Bracteantha Anderb.; Bradburia Torr. & A. Gray; Brickellia Elliott—brickellbush; Brickelliastrum R. M. King & H. Rob.—brickellbush; Brintonia—mock goldenrod; Bryomorphe Harv.; Buphthalmum L.; Burkartia Crisci; Cabreriella Cuatrec.; Cacalia L.—Indian plantain; Cacaliopsis A. Gray; Cacosmia Kunth; Caesulia Roxb.; Calea L.; Calendula L.—marigold; Callicephalus C. A. Mey.; Callilepis DC.; Callistephus Cass.; Calocephalus R. Br.; Calomeria Vent.; Calopappus Meyen; Calorezia Panero; Calostephane Benth.; Calotesta P. O. Karis; Calotis R. Br.; Calycadenia DC—western rosinweed; Calycocorsus F. W. Schmidt; Calycoseris A. Gray—tackstem; Calyptocarpus Less.; Camchaya Gagnep.; Campovassouria R. M. King & H. Rob.; Camptacra N. T. Burb.; Campuloclinium DC; Canadanthus—mountain aster (?); Cancrinia Kar. & Kir.; Cancriniella Tzvelev; Cardopatium Juss.; Carduncellus Adans.; Carduus L.—plumeless thistle; Carlina L.—carline thistle; Carminatia Moc. ex DC.; Carpesium L.; Carphephorus Cass.—chaffhead; Carphochaete A. Gray—bristlehead; Carramboa Cuatrec.; Carterothamnus R. M. King; Carthamus L.—distaff thistle; Cassinia R. Br.; Castalis Cass.; Castenedia R. M. King & H. Rob.; Catamixis Thomson; Catananche L.; Catatia Humbert; Catolesia; Caucasalia; Cavalcantia R. M. King & H. Rob.; Cavea W. W. Sm. & Small; Caxamarca; Celmisia Cass.; Centaurea L.—knapweed, cornflower, star thistle; Centaurodendron Johow; Centauropsis Bojer ex DC.; Centaurothamnus Wagenitz & Dittrich; Centipeda Lour; Centratherum Cass.; Cephalipterum A. Gray; Cephalopappus Nees & Mart.; Cephalorrhynchus Boiss.; Cephalosorus A. Gray; Ceratogyne Turcz.; Ceruana Forssk.; Chacoa R. M. King & H. Rob.; Chaenactis DC—pincushion; chaetadelpha A. Gray ex S.Watson—skeletonweed; Chaetanthera Ruiz & Pav.; Chaetopappa DC—least daisy; Chaetospira S. F. Blake; Chaetymenia Hook. & Am.; Chamaechaenactis Rydb.; Chamaegeron Schrenk; Chamaeleon Cass.; Chamaemelum Mill.—dogfennel; Chamomilla—chamomilla, pineapple weed (synonym of Matricaria L.); Chaptalia Vent.—sunbonnetts; Chardinia Desf.; Cheirolophus Cass.; Chersodoma Phil.; Chevreulia Cass.; Chiliadenus Cass.; Chiliocephalum Benth.; Chiliophyllum Phil.; Chiliotrichiopsis Cabrera; Chiliotrichum Cass.; Chimantaea Maguire, Steyerm. & Wurdack; Chionolaena DC.; Chionopappus Benth; Chlamydophora Ehrenb. ex Less.; Chloracantha—G. L. Nesom; Chondrilla L.; Chondropyxis D. A. Cooke; Chorisis DC.; Chresta Veil. ex DC.; Chromolaena DC—thoroughwort; Chromolepis Benth.; Chronopappus DC.; Chrysactinia A. Gray; Chrysactinium (Kunth) Wedd.; Chrysanthellum Rich.; Chrysanthemoides Fabr.; Chrysanthemum L.; Chrysocephalum Walp.; Chrysocoma L.; Chrysogonum L.; Chrysoma Nutt.; Chrysophthalmum Sch.Bip. ex Walp.; Chrysopsis (Nutt.) Elliott—goldenaster; Chrysothamnus Nutt.—rabbitbrush; Chthonocephalus Steetz; Chucoa Cabrera; Chuquiraga Juss.; Chyrsactinia; Cicerbita Wallr.; Ciceronia Urb.; Cichorium L.—chicory; Cineraria L.; Cirsium Mill.—thistle; Cissampelopsis (DC.) Miq.; Cladanthus Cass.; Cladochaeta DC.; Clappia A. Gray—clapdaisy; Clibadium L.; Cnicothamnus Griseb.; Cnicus L.—blessed thistle; Coespeletia Cuatrec.; Coleocoma F.Muell.; Coleostephus Cass.; Colobanthera Humbert; Columbiadoria G. L. Nesom; Comaclinium Scheidw. & Planch.; Comborhiza; Commidendrum DC.; Complaya Strother; Condylidium; Conoclinium R. M. King & H. Rob.—thoroughwort, mistflower; Condylopodium R. M. King & H. Rob.; Conocliniopsis R. M. King & H. Rob.; Conoclinium DC.; Conyza Less.—horseweed; Coreocarpus Benth.; Coreopsis L.—tickseed; Corethamnium R. M. King & H. Rob.; Corethrogyne DC—sandaster; Coronidium Paul G. Wilson; Correllia A. M. Powell; Corymbium L.; Cosmos Cas.; Cotula L.—waterbuttons; Coulterella Vasey & Rose; Cousinia Cass.; Cousiniopsis Nevski; Craspedia G. Forst.; Crassocephalum Moench—ragleaf; Cratystylis S.Moore; Cremanthodium Benth.; Crepidiastrum Nakai; Crepis L.—hawksbeard; Crinitaria; Critonia P.Browne—thoroughwort; Critoniadelphus R. M. King & H. Rob.; Critoniella R. M. King & H. Rob.; Critoniopsis Sch.Bip.; Crocidium Hook—spring-gold; Cronquistia R. M. King; Cronquistianthus R. M. King & H. Rob.; Croptilon Raf.—scratchdaisy; Crossostephium Less.; Crossothamnus R. M. King & H. Rob.; Crupina (Pers.) DC.; Cuatrecasanthus; Cuatrecasasiella H. Rob.; Cuchumatanea Seid. & Beaman; Cullumia R. Br.; Cuniculotinus; Cuspidia Gaertn.; Cyanopsis—knapweed (synonym of Volutaria Cass.); Cyanthillium—ironweed (plant) (synonym of Vernonia Schreb.); Cyathocline Cass.; Cyathomone S. F. Blake; Cyclachaena Fresen. ex Schltdl.; Cyclolepis Gillies ex D.Don; Cylindrocline Cass.; Cymbolaena Smoljan.; Cymbonotus Cass.; Cymbopappus B. Nord.; Cymophora B. L. Rob.; Cynara L.—artichoke; Dacryotrichia Wild; Dahlia Cav.; Damnamenia; Damnxanthodium Strother; Darwiniothamnus Harling; Dasycondylus R. M. King & H. Rob.; Dasyphyllum Kunth; Daveaua Willk. ex Mariz; Decachaeta DC.; Decastylocarpus Humbert; Decazesia F. Muell.; Deinandra—often included in Hemizona; Delairea Lem.—capeivy; Delamerea S. Moore; Delilia Spreng.; Dendranthema (DC.) Des Moul.—arctic daisy; Dendrocacalia (Nakai) Tuyama; Dendrophorbium (Cuatrec.) C. Jeffrey; Dendrosenecio (Hauman ex Humbert) B.Nord.; Dendroseris D. Don; Denekia Thunb.; Desmanthodium Benth.; Dewildemania O. Hoffm.; Diacranthera R. M. King & H. Rob.; Dianthoseris Sch.Bip.; Diaperia Nutt.; Diaphractanthus Humbert; Dicercoclados C. Jeffrey & Y. L. Chen; Dichaetophora A. Gray; Dichrocephala L′Her. ex DC.; Dichromochlamys Dunlop; Dicoma Cass.; Dicoria Torr. & A. Gray—twinbugs; Dicranocarpus A. Gray; Didelta L′Her.; Dielitzia P. S. Short; Dieteria; Digitacalia Pippen; Dimeresia A. Gray; Dimerostemma Cass.; Dimorphocoma F. Muell. & Tate; Dimorphotheca Moench—cape marigold; Dinoseris Griseb.; Diodontium F. Muell.; Diplazoptilon Y. Ling; Diplostephium Kunth; Dipterocome Fisch. & C. A. Mey.; Dipterocypsela S. F. Blake; Disparago Gaertn.; Dissothrix A. Gray; Distephanus (Cass.) Cass.; Disynaphia Hook. & Am. ex DC.; Dithyrostegia A. Gray; Dittrichia Greuter; Doellingeria Ness.—whitetop; Dolichlasium Lag.; Dolichoglottis B. Nord.; Dolichorrhiza (Pojark.) Galushko; Dolichothrix Hilliard & B. L. Burtt; Dolomiaea DC.; Doniophyton Wedd.; Doronicum L.—false leopardbane; Dracopis—coneflower (synonym of Rudbeckia L.); Dresslerothamnus H. Rob.; Dubautia Gaudich.; Dubyaea DC.; Dugaldia (Cass.) Cass.; Dugesia A. Gray; Duhaldea DC.; Duidaea S. F. Blake; Duseniella K. Schum.; Dymondia Compton; Dyscritogyne R. M. King & H. Rob.; Dyscritothamnus B. L. Rob; Dysodiopsis (A. Gray) Rydb.—dogfennel; Dyssodia Cay.—dogweed; Eastwoodia Brandegee; Eatonella A. Gray; Echinacea Moench—coneflower; Echinops L.—globethistle; Eclipta L.; Edmondia Cass.; Egletes Cass.—tropic daisy; Eitenia R. M. King & H. Rob.; Ekmania Gleason; Elachanthus F. Muell.; Elaphandra Strother; Elephantopus L.—elephantsfoot; Eleutheranthera Poit. ex Bosc; Ellenbergia Cuatrec.; Elytropappus Cass.; Emilia (Cass.) Cass.—tasselflower; Emiliella S. Moore; Encelia Adans.—brittlebush; Enceliopsis (A. Gray) A. Nelson—sunray; Endocellion Turcz. ex Herder; Endopappus Sch.Bip.; Engelmannia A. Gray ex Nutt.—Engelmann's daisy; Engleria O. Hoffm.; Enydra Lour—swampwort; Epaltes Cass.; Epilasia (Bunge) Benth.; Episcothamnus H. Rob.; Epitriche Turcz.; Erato DC.; Erechtites Raf—burnweed; Eremanthus Less; Eremosis (DC.) Gleason; Eremothamnus O. Hoffm.; Eriachaenium Sch.Bip.; Ericameria Nutt.—goldenbush, heath goldenrod; Ericentrodea S. F. Blake & Sherff; Erigeron L.—daisy, fleabane; Eriocephalus L.; Eriochlamys Sond. & F. Muell.; Eriophyllum Lag.—woolly sunflower; Eriotrix Cass.; Erlangea Sch.Bip.; Erodiophyllum F. Muell.; Erymophyllum Paul G.Wilson; Eryngiophyllum Greenm.; Erythradenia (B. L. Rob.) R. M. King & H. Rob.; Erythrocephalum Benth.; Espejoa DC.; Espeletia Mutis ex Humb. & Bonpl.—frailejones, Venezuela, Colombia, Ecuador.; Espeletiopsis Cuatrec.; Ethulia L.f; Eucephalus—Cascade Aster, eucephalus (?); Euchiton Cass.—cudweed; Eumorphia DC; Eupatoriastrum Greenm.; Eupatorina R. M. King & H. Rob.; Eupatoriopsis Hieron.; Eupatorium L.—thoroughwort, snakeweed; Euphrosyne DC; Eurybia—Nees aster (?); Eurydochus Maguire & Wurdack; Euryops (Cass.) Cass.; Eutetras A. Gray; Euthamia (Nutt.) Elliott—goldentop; Eutrochium—Joe-Pye weed; Evacidium Pomel; Evax—pygmy cudweed (synonym of Filago L.); Ewartia Beauverd; Ewartiothamnus Anderb.; Exomiocarpon Lawalree; Faberia Hemsl.; Facelis Hemsl.—trampweed; Farfugium Lindl.; Faujasia Cass.; Faxonia Brandegee; Feddea Urb.; Feldstonia P. S. Short; Felicia Cass.; Femeniasia Susanna; Fenixia Merr.; Ferreyranthus H. Rob. & Brettell; Ferreyrella S. F. Blake; Filago L.—cottonrose; Filifolium Kitam.; Fitchia Hook.f.; Fitzwillia P. S. Short; Flaveria Juss.—yellowtops; Fleischmannia Sch.Bip.—thoroughwort; Fleischmanniopsis R. M. King & H. Rob.; Florestina Cass.; Floscaldasia Cuatrec.; Flosmutisia Cuatrec.; Flourensia DC—tarwort; Flyriella R. M. King & H. Rob.—brickellbush; Formania W. W. Sm. & Small; Foveolina Kallersjo; Fulcaldea Poir.; Gaillardia Foug.—blanketflower; Galactites Moench; Galatella; Galeana La Llave; Galeomma Rauschert; Galinsoga Ruiz & Pay.—gallant-soldier; Gamochaeta Wedd.—everlasting; Gamochaetopsis Anderb. & Freire; Garberia A. Gray; Garcibarrigoa Cuatrec.; Garcilassa Poepp.; Gardnerina R. M. King & H. Rob.; Garuleum Cass.; Gazania Gaertn.; Geigeria Griess.; Geissolepis B. L. Rob.; Geissopappus Benth.; Geraea Torr. & A. Gray—desert sunflower; Gerbera L.—Gerbera or Transvaal daisy; Geropogon L.; Gibbaria Cass.; Gilberta Turcz.; Gilruthia Ewart; Gladiopappus Humbert; Glossarion Maguire & Wurdack; Glossocardia Cass.; Glossopappus Kunze; Glyptopleura Eaton; Gnaphaliothamnus Kirp.; Gnaphalium L.—cudweed; Gnephosis Cass.; Gochnatia Kunth; Goldmanella Greenm.; Gongrostylus R. M. King & H. Rob.; Gongylolepis R. H. Schomb.; Goniocaulon Cass.; Gonospermum Less.; Gorceixia Baker; Gorteria L.; Gossweilera S.Moore; Goyazianthus R. M. King & H. Rob.; Grangea Adans.; Grangeopsis Humbert; Graphistylis B. Nord.; Gratwickia F. Muell.; Grauanthus Fayed; Grazielia R. M. King & H. Rob.; Greenmaniella W. M. Sharp; Grindelia Willd.—gumweed; Grisebachianthus R. M. King & H. Rob.; Grosvenoria R. M. King & H. Rob.; Guardiola Cerv. ex Humb. & Bonpl.; Guayania R. M. King & H. Rob.; Guevaria R. M. King & H. Rob.; Guizotia Cass.; Gundelia L.; Gundlachia A. Gray; Gutenbergia Sch.Bip.; Gutierrezia Lag.—snakeweed; Gymnarrhena Desf.; Gymnocondylus R. M. King & H. Rob.; Gymnocoronis DC.; Gymnodiscus Less.; Gymnolaena(DC.) Rydb.; Gymnosperma Benth.; Gymnostephium Less.; Gymnostyles —burrweed (synonym of Soliva Ruiz & Pay.); Gynoxys Cass.; Gynura Cass.; Gypothamnium Phil.; Gyptidium R. M. King & H. Rob.; Gyptis (Cass.) Cass.; Gyrodoma Wild; Haastia Hook.f.; Haeckeria F. Muell.; Haegiela P. S. Short; Handelia Heimerl; Haplocalymma S. F. Blake; Haplocarpha Less.—onefruit; Haploesthes A. Gray—false broomweed; Haplopappus Cass.; Haplostephium Mart. ex DC.; Harleya S. F. Blake; Harmonia; Harnackia Urb.; Hartwrightia A. Gray ex S. Watson; Hasteola Raf.—false Indian plantain; Hatschbachiella R. M. King & H. Rob.; Hazardia Greene—bristleweed; Hebeclinium DC—thoroughwort; Hecastocleis A. Gray; Hedypnois Mill.; Helenium L.—sneezeweed; Helianthella Torr. & A. Gray; Helianthopsis H. Rob.; Helianthus L.—sunflowers; Helichrysopsis Kirp.; Helichrysum Mill.—strawflower,everlasting; Heliocauta Humphries; Heliomeris Nutt.—false goldeneye; Heliopsis Pers.; Helminthia (synonym of Picris L.); Helminthotheca (obsolete); Helogyne Nutt.; Hemisteptia Fisch. & C. A. Mey.; Hemizonia DC—tarweed; Henricksonia B. L.Turner; Heptanthus Griseb.; Herderia Cass.; Herodotia Urb. & Ekman; Herrickia (synonym of Aster L.); Hertia; Hesperevax—dwarf-cudweed (?); Hesperodoria—glowweed (?); Hesperomannia A. Gray—island-aster; Heteracia Fisch. & C. A. Mey.; Heteranthemis Schott—oxeye; Heterocoma DC.; Heterocondylus R. M. King & H. Rob.; Heterocypsela H. Rob.; Heteroderis (Bunge) Boiss.; Heterolepis Cass.; Heteromera Pomel; Heteromma Benth.; Heteropappus Less.; Heteroplexis C. C. Chang; Heterorhachis Sch.Bip. ex Walp.; Heterosperma Cay.; Heterothalamus Less.; Heterotheca Cass.—false goldenaster, telegraph plant; Hidalgoa La Llave; Hieracium L.—hawkweed; Hilliardia B. Nord.; Hinterhubera Sch.Bip. ex Wedd.; Hippia L.; Hippolytia Poljakov; Hirpicium Cass.; Hispidella Barnadez ex Lam.; Hoehnephytum Cabrera; Hoffmanniella Schltr. ex Lawalree; Hofmeisteria Walp.; Holocarpha Greene—tarweed; Holocheilus Cass.; Hololeion Kitam; Holozonia Greene; Homogyne Cass.; Hoplophyllum DC.; Huarpea Cabrera; Hubertia Bory; Hughesia R. M. King & H. Rob.; Hulsea Torr. & A. Gray—alpinegold; Humeocline Anderb.; Hyalis D. Don ex Hook. & Arn.; Hyalochaete Dittrich & Rech.f.; Hyalochlamys A. Gray; Hyaloseris Griseb.; Hyalosperma Steetz; Hybridella Cass.; Hydrodyssodia B. L. Turner; Hydroidea P. O. Karis; Hydropectis Rydb.; Hymenocephalus Jaub. & Spach; Hymenoclea Torr. & A. Gray—burrobrush, burrobush; Hymenolepis Cass.; Hymenonema Cass.; Hymenopappus L′Her; Hymenostemma Kunze ex Willk.; Hymenostephium Benth.; Hymenothrix A. Gray—thimblehead; Hymenoxys Cass.—rubberweed; HyoserisL.; Hypacanthium Juz.; Hypelichrysum Kirp.; Hypericophyllum Steetz; Hypochaeris L.—catsear; Hysterionica Willd.; Hystrichophora Mattf.; Ichthyothere Mart.; Idiothamnus R. M. King & H. Rob.; Ifloga Cass.; Ighermia Wiklund; Iltisia S. F. Blake; Imeria R. M. King & H. Rob.; Inezia E. Phillips; Inula L.—yellowhead; Inulanthera Kallersjo; Ionactis—Stiff-leaved Asters (?.); Iocenes B. Nord.; Iodocephalus Thorel ex Gagnep.; Iogeton Strother; Iostephane Benth.; Iphiona Cass.; Iphionopsis Anderb.; Iranecio B. Nord.; Irwinia Barroso; Ischnea F. Muell.; Isocarpha R. Br.—pearlhead; Isocoma Nutt.—goldenbush, jimmyweed; Isoetopsis Turcz.; Isopappus Torr. & A. Gray; Isostigma Less.; Iva L.—marshelder, sumpweed; Ixeridium (A. Gray) Tzvelev; Ixeris (Cass.) Cass.; Ixiochlamys F. Muell. & Sond.; Ixiolaena Benth.; Ixodia R. Br.; Jacmaia B. Nord.; Jaegeria Kunth; Jalcophila Dillon & Sagast.; Jaliscoa S. Watson; Jamesianthus S. F. Blake & Sherff; Jaramilloa R. M. King & H. Rob.; Jasonia (Cass.) Cass.; Jaumea Pers.; Jefea Strother; Jeffreya Wild; Jensia; Joseanthus; Jungia L.f.; Jurinea Cass.; Kalimeris (Cass.) Cass.—aster; Karelinia Less.; Karvandarina Rech.f.; Kaschgaria Poljakov; Kaunia R. M. King & H. Rob.; Kemulariella; Keysseria Lauterb.; Kinghamia C. Jeffrey; Kingianthus H. Rob.; Kippistia F. Muell.; Kirkianella Allan; Kleinia Mill.; Koanophyllon Arruda—thoroughwort; Koehneola Urb.; Koelpinia Pall.; Koyamacalia (?); Krigia Schreb—dwarf dandelion; Kyrsteniopsis R. M. King & H. Rob; Lachanodes DC.; Lachnophyllum Bunge; Lachnorhiza A. Rich.; Lachnospermum Willd.; Lactuca L.—lettuce; Lactucosonchus (Sch.Bip.) Svent.; Laennecia Cass.—laennecia, laennicia; Laestadia Kunth ex Less.; Lagascea Cay.; Lagenophora Cass.—island-daisy; Laggera Sch.Bip. ex Benth.; Lagophylla Nutt.—hareleaf; Lamprachaenium Benth.; Lamprocephalus B. Nord.; Lamyropappus Knorring & Tamamsch.; Lamyropsis (Kharadze) Dittrich; Langebergia Anderb.; Lantanopsis C. Wright; Lapsana L.—nipplewort; Lapsanastrum; Lasianthaea DC; Lasiocephalus Schltdl.; Lasiolaena R. M. King & H. Rob.; Lasiopogon Cass.; Lasiospermum Lag.—cocoonhead; LastheniaCass.—goldfield; Launaea Cass.—aulaga; Lawrencella Lindl.; Layia Hook. & Am. ex DC—tidytips; Lecocarpus Decne.; Leibnitzia Cass.—sunbonnets; Leiboldia Schltdl. ex Gleason; Leiocarpa; Lepidaploa; Lembertia Greene; Lemooria P. S. Short; Leontodon—hawkbit; Leontopodium (Pers.) R. Br. ex Cass.—edelweiss; Lepidesmia Klatt; Lepidolopha C. Winkl.; Lepidolopsis Poljakov; Lepidonia S. F. Blake; Lepidophorum Neck. ex DC.; Lepidophyllum Cass.; Lepidospartum (A. Gray) A. Gray—broomsage; Lepidostephium Oliv.; Leptinella Cass.—Brass Buttons, Creeping Cotula; Leptocarpha DC.; Leptoclinium (Nutt.) Benth.; Leptorhynchos Less.—scaly button; Leptotriche Turcz.; Lescaillea Griseb.; Lessingia Cham.—vinegarweed; Leucactinia Rydb.; Leucanthemella Tzvelev; Leucanthemopsis (Giroux) Heywood; Leucanthemum Mill.—daisy, Oxeye daisy; Leucheria Lag.; Leucochrysum—sunray (?); Leucomeris; Leucophyta; Leucopsis (DC.) Baker; Leucoptera B. Nord; Leunisia Phil.; Leuzea DC.; Leysera L.; Liabum Adans.; Liatris Liabum Adans.—blazing star, gay feather; Libanothamnus Ernst; Lidbeckia Bergius; Lifago Schweinf. & Muschl.; Ligularia Cass.; Ligulariopsis; Limbarda Adans.; Lindheimera A. Gray & Engelm.; Lipochaeta DC—nehe; Lipskyella Juz.; Litothamnus R. M. King & H. Rob.; Litrisa Small; Llerasia Triana; Logfia Cass.—cottonrose; Lomatozona Baker; Lonas Adans.; Lopholaena DC.; Lophopappus Rusby; Lorandersonia; Lordhowea B. Nord.; Lorentzianthus R. M. King & H. Rob.; Loricaria Wedd.; Lourteigia R. M. King & H. Rob.; Loxothysanus B. L. Rob.; Lucilia Cass.; Luciliocline Anderb. & Freire; Lugoa DC.; Luina Benth.—silverback; Lulia Zardini; Lundeffianthus H. Rob.; Lycapsus Phil.; Lychnophora Mart.; LycoserisCass.; Lygodesmia D. Don—skeleton weed; Macdougalia A. Heller; Machaeranthera Nees—goldenweed, tansyaster; Macowania Oliv.; Macrachaenium Hook.f.; Macraea Hook.f.; Macroclinidium Maxim.; Macronema Nutt.=Ericameria Nutt.; Macropodina R. M. King & H. Rob.; Macvaughiella R. M. King & H. Rob.; Madia Molina—tarweed; Mairia Nees; Malacothrix DC—desert dandelion; Malmeanthus R. M. King & H. Rob.; Malperia S.Watson; Mantisalca Cass.; Marasmodes DC.; Marshallia Schreb.—Barbara's buttons; Marshalljohnstonia Henr.; Marticorenia Crisci; Matricaria L.—mayweed; Mattfeldanthus H. Rob. & R. M. King; Mattfeldia Urb.; Matudina R. M. King & H. Rob.; Mauranthemum Vogt & Oberpr.; Mausolea Poljakov; Mecomischus Coss. ex Benth.; Megalodonta Greene—watermarigold; Melampodium L.—blackfoot; Melanodendron DC.; Melanthera Rohr—squarestem; Metalasia R. Br.; Metastevia Grashoff; Mexerion G. L. Nesom; Mexianthus B. L. Rob.; Micractis DC.; Microcephala Pobed.; Microglossa DC.; Microgynella Grau; Microliabum Cabrera; Micropus L.—cottonseed; Microseris D. Don—silverpuffs, yam daisy; Microspermum Lag.; Mikania Willd.—hempvine; Mikaniopsis Milne-Redh.; Miliaria L.; Millotia Cass.; Minuria DC.; Miricacalia Kitam.; Misbrookia; Miyamayomena; Mniodes (A. Gray) Benth.; Monactis Kunth; Monoculus; Monarrhenus Cass.; Monenteles Labill.; Monogereion G. M. Barroso & R. M. King; Monolopia DC; Monopholis S. F.Blake; Monoptilon Torr. & A. Gray—desertstar; Montanoa Cerv.; Moonia Arn.; Moquinia DC.; Morithamnus R. M. King, H. Rob. & G. M.Barroso; Moscharia Ruiz & Pay.; Msuata O. Hoffm.; Mulgedium Cass.; Munnozia Ruiz & Pay.; Munzothamnus Raven; Muschleria S. Moore; Mutisia L.f.; Mycelis cass.; Myopordon Boiss.; Myriactis Less.; Myriocephalus Benth.; Myripnois Bunge; Myxopappus Kallersjo; Nabalus Cass.; Nananthea DC.; Nannoglottis Maxim.; Nanothamnus Thomson; Nardophyllum (Hook. & Am.) Hook. & Arn.; Narvalina Cass.; Nassauvia Comm. ex Juss.; Nauplius (Cass.) Cass.; Neblinaea Maguire & Wurdack; Nelsonianthus H. Rob. & Brettell; Nemosenecio (Kitam.) B. Nord.; Neocabreria R. M. King & H. Rob.; Neocuatrecasia R. M. King & H. Rob.; Neohintonia R. M. King & H. Rob.; Neojeffreya Cabrera; Neomirandea R. M. King & H. Rob.; Neonesomia; Neopallasia Poljakov; Neotysonia Dalla Torre & Harms; Nesomia; Nestlera; Nestotus; Neurolaena R. Br.; Neurolakis Mattf.; Nicolasia S. Moore; Nicolletia A. Gray—hole-in-the-sand; Nidorella Cass.; Nikitinia Iljin; Nipponanthemum (Kitam.) Kitam.; Nolletia Cass.; Nothobaccharis R. M. King & H. Rob.; Nothocalais Greene—prairie-dandelion; Noticastrum DC.; Notobasis (Cass.) Cass.—Syrian thistle; Notoptera Urb.; Notoseris C. Shih; Nouelia Franch.; Novenia Freire; Oaxacania B. L. Rob. & Greenm.; Oblivia Strother; Ochrocephala Dittrich; Oclemena—aster (synonym of Aster L.); Odixia Orchard; Odontocline B. Nord.; Odixia Orchard; Odontocline B. Nord.; Oedera L.; Oldenburgia Less.; Olearia Moench—daisy bush; Olgaea Iljin; Oligactis (Kunth) Cass.; Oliganthes Cass.; Oligocarpus Less.; Oligochaeta (DC.) K. Koch; Oligoneuron Small—goldenrod (?); Oligothrix DC.; Olivaea Sch.Bip. ex Benth.; Omalotheca Cass.—arctic cudweed; Omphalopappus O. Hoffm.; Oncosiphon Kallersjo; Ondetia Benth.; Onopordum L.—Cotton thistle; Onoseris Willd.; Oonopsis (Nutt.) Greene—false goldenweed; Oparanthus Sherff; Ophryosporus Meyen; Opisthopappus C. Shih; Oreochrysum Rydb.—goldenrod; Oreoleysera Bremer; Oreostemma—aster (synonym of Aster L.); Oritrophium (Kunth) Cuatrec.; Orochaenactis Coville; Osbertia Greene; Osmadenia Nutt. (?); Osmiopsis R. M. King & H. Rob.; Osmitopsis Cass.; Osteospermum L.—daisybush; Otanthus Hoffmanns. & Link; Oteiza La Llave; Othonna L.; Otopappus Benth.; Otospermum Willk.; Outreya Jaub. & Spach; Oxycarpha S. F. Blake; Oxylaena Benth. ex Anderb.; Oxylobus (Moq. ex DC.) A. Gray; Oxypappus Benth.; Oxyphyllum Phil.; Oyedaea DC.; Ozothamnus R. Br.; Pachylaena D. Don ex Hook. & Am.; Pachystegia Cheeeseman; Pachythamnus (R. M. King & H. Rob.) R. M. King & H. Rob.; Packera A. Love & D. Love—(includes some plants formerly Senecio); Pacourina Aubl.; Palaeocyanus Dostal; Palafoxia Lag.—palafox; Paleaepappus Cabrera; Pallenis Cass. (synonym of Asteriscus); Pamphalea Lag.; Pappobolus S. F. Blake; Papuacalia Veldkamp; Paracalia Cuatrec.; Paragynoxys (Cuatrec.) Cuatrec.; Paraixeris Nakai; Paranephelius Poepp.; Parantennaria Beauverd; Parapiqueria R. M. King & H. Rob.; Paraprenanthes C. C. Chang ex C. Shih; Parasenecio W. W. Sm. & Small—Indian plantain; Parastrephia Nutt.; Parthenice A. Gray; Parthenium L.—feverfew, guayule; Pasaccardoa Kuntze; Pascalia Ortega (synonym of Wedelia); Pechuel-Ioeschea O. Hoffm.; Pectis L.—cinchweed, fetid marigold; Pegolettia Cass.; Pelucha S. Watson; Pentacalia Cass.; Pentachaeta Nutt.—pygmy daisy; Pentanema Cass.; Pentatrichia Klatt; Pentzia Thunb.; Perdicium L.; Perezia Lag.; Pericallis D. Don—includes Florist's Cineraria; Pericome A. Gray; Peripleura Clifford & Ludlow; Perityle Benth.—rock daisy; Perralderia Coss.; Pertya Sch.Bip.; Perymeniopsis H. Rob.; Perymenium Schrad.; Petalacte D. Don; Petasites Mill.—butterbur; Peteravenia R. M. King & H. Rob.; Petradoria Greene—rock goldenrod; Petrobium R. Br.; Peucephyllum A. Gray; Peyrousea DC.; Phacellothrix F. Muell.; Phaenocoma D. Don; Phaeostigma Muldashev; Phagnalon Cass.; Phalacrachena Iljin; Phalacraea DC.; Phalacrocarpum (DC.) Willk.; Phalacroseris A. Gray—mock dandelion; Phaneroglossa B. Nord.; Phanerostylis (A. Gray) R. M. King & H. Rob.; Phania DC.; Philactis Schrad.; Philoglossa DC.; Philyrophyllum O. Hoffm.; Phoebanthus S. F. Blake—false sunflower; Phyllocephalum Blume; Phymaspermum Less.; Picnomon Adans.; Picradeniopsis—bahia (synonym of Bahia Lag.); Picris L.—oxtongue; Picrosia D. Don; Picrothamnus Nutt.—bud sagebrush; Pilosella Hill; Pilostemon Iljin; Pinaropappus Less.—rock lettuce; Piora J. Kost.; Pippenalia McVaugh; Piptocarpha R. Br.—ash daisy; Piptocoma Cass.—velvetshrub; Piptolepis Sch.Bip.; Piptothrix A. Gray; Piqueria Cay.; Piqueriella R. M. King & H. Rob.; Piqueriopsis R. M. King; Pithecoseris Mart. ex DC.; Pithocarpa Lindl.; Pittocaulon H. Rob. & Brettell; Pityopsis Nutt.—silkgrass; Pladaroxylon (Endl.) Hook.f.; Plagiobasis Schrenk; Plagiocheilus Arn. ex DC.; Plagiolophus Greenm.; Plagius L′Her. ex DC.; Planaltoa Taub.; Planea P. O. Karis; Plateilema (A. Gray) Cockerell; Platycarpha Less.; Platypodanthera R. M. King & H. Rob.; Platyschkuhria (A. Gray) Rydb.—basin daisy; Plazia Ruiz & Pay.; Plecostachys Hilliard & B. L. Burtt; Plectocephalus D. Don; Pleiotaxis Steetz; Pleurocarpaea Benth.; Pleurocoronis Pleurocarpaea Benth.; Pleuropappus F. Muell.; Pleurophyllum Hook.f.; Pluchea Cass.—camphorweed, fleabane; Plummera A. Gray; Podachaenium Benth. ex Oerst.; Podanthus Lag.; Podocoma Cass.; Podolepis Labill.; Podotheca Cass.; Poecilolepis Grau; Pogonolepis Steetz; Pojarkovia Askerova; Pollalesta Kunth; Polyachyrus Lag.; Polyanthina R. M. King & H. Rob.; Polyarrhena Cass.; Polycalymma F. Muell. & Sond.; Polychrysum (Tzvelev) Kovalevsk.; Polymnia L.; Porophyllum Adans.—poreleaf; Porphyrostemma Benth. ex Oliv.; Praxeliopsis G. M. Barroso; Praxelis Cass.; Prenanthella Rydb.; Prenanthes L.—rattlesnakeroot; Printzia Cass.; Prionopsis Nutt.; Prolobus R. M. King & H. Rob.; Prolongoa Boiss.; Proteopsis Mart. & Zucc. ex Sch.Bip.; Proustia Lag.; Psacaliopsis H. Rob. & Brettell; Psacalium Cass.—Indianbush; Psathyrotes A. Gray—turtleback, i.e. turtleback (plant); Pseudelephantopus Rohr—dog's-tongue (synonym of Elephantopus L.); Pseudobaccharis Cabrera; Pseudobahia (A. Gray) Rydb.—sunburst; Pseudoblepharispermum J.-P.Lebrun & Stork; Pseudobrickellia R. M. King & H. Rob.; Pseudocadiscus Lisowski; Pseudoclappia Rydb.—false clapdaisy; Pseudognaphalium Kirp.—false cudweed, cudweed; Pseudogynoxys (Greenm.) Cabrera; Pseudohandelia Tzvelev; Pseudokyrsteniopsis R. M. King & H. Rob.; Pseudonoseris H. Rob. & Brettell; Pseudostifftia H. Rob.; Psiadia Jacq.; Psiadiella Humbert; Psilactis A. Gray—tansyaster; Psilocarphus Nutt.—woollyheads; Psilostrophe DC—paperflower; Psychrogeton Boiss.; Psychrophyton Beauverd; Pterachenia (Benth.) Lipsch.; Pterocaulon Elliott—blackroot; Pterocaulon Elliott; Pterocypsela C. Shih; Pteronia L.; Pterothrix DC.; Pterygopappus Hook.f.; Ptilostemon Cass.; Pulicaria Gaertn.—false fleabane; Pycnocephalum (Less.) DC.; Pyrrhopappus DC—desert chicory; Pyrrocoma Hook—goldenweed; Pycnosorus—billy buttons; Quelchia N. E. Br.; Quinetia Cass.; Quinqueremulus Paul G. Wilson; Radlkoferotoma Kuntze; Rafinesquia Nutt.—California chicory; Raillardella—silvermat (?); Raillardiopsis Rydb.; Rainiera Greene; Raoulia Hook.f. ex Raoul—Vegetable Sheep, Mat Daisy; Raouliopsis S. F. Blake; Rastrophyllum Wild & G. V. Pope; Ratibida Raf.—prairie coneflower; Raulinoreitzia R. M. King & H. Rob.; Rayjacksonia—tansyaster (?); Reichardia Roth.—brighteyes; Relhania L′Her.; Remya W. F. Hillebr. ex Benth.; Rennera Merxm.; Rensonia S. F. Blake; Revealia R. M. King & H. Rob.; Rhagadiolus Scop.; Rhamphogyne S. Moore; Rhanteriopsis Rauschert; Rhanterium Desf.; Rhodanthe Lindl.—sunray; Rhodogeron Griseb.; Rhynchopsidium; Rhynchospermum Reinw.; Rhysolepis S. F. Blake; Richteria Kar. & Kir.; Riencourtia Cass.; Rigiopappus A. Gray; Robinsonia DC.; Rochonia DC.; Rojasianthe Standl. & Steyerm; Rolandra Rottb.—yerba de plata; Roldana La Llave—groundsel; Rosenia Thunb.; Rothmaleria Font Quer; Rudbeckia L.—coneflower; Rugelia Shuttlew. ex Chapm.—Rugel's Indian plantain; Ruilopezia Cuatrec.; Rumfordia DC.; Russowia C. Winkl.; Rutidosis DC.; Sabazia Cass.; Sachsia Griseb.; Salmea DC—bejuco de miel; Salmeopsis Benth.; Santoline L.—lavender cotton; Santosia R. M. King & H. Rob.; Sanvitalia Lam.—creeping zinnia; Sartorina R. M. King & H. Rob.; Sartwellia A. Gray—glowwort; Saussurea DC—saw-wort; Scalesia Arn.; Scariola F. W. Schmidt; Scherya R. M. King & H. Rob.; Schischkinia Iljin; Schistocarpha Less.; Schistostephium Less.; Schizogyne Cass.; Schizoptera Turcz.; Schizotrichia Benth.; Schkuhria Roth—false threadleaf; Schlechtendalia Less.; Schmalhausenia C. Winkl.; Schoenia Steetz; Schumeria Iljin; Sciadocephala Mattf.; Sclerocarpus Jacq.—bonebract; Sclerolepis Cass.—bogbutton; Sclerorhachis (Rech.f.) Rech.f.; Sclerostephane Chiov.; Scolymus L.—golden thistle; Scorzonella Nutt.; Scorzonera L.—[salsify]; Scrobicaria Cass.; Scyphocoronis A. Gray; Selleophytum Urb.; Selloa Kunth; Semiria; Senecio L.—groundsel, ragwort; Sericocarpus Nees—whitetop aster; Seriphidium (Besser) Poljak.; Serratula L.—plumeless saw-wort; Shafera Greenm.; Sheareria S. Moore; Shinnersia R. M. King & H. Rob.; Shinnersoseris Tomb—beaked skeletonweed; Siapaea; Siebera J. Gay; Sigesbeckia L.—St. Paul's wort; Siloxerus Labill.; Silphium L.—rosinweed; Silybum Adans.—milk thistle; Simsia Pers.—bush sunflower; Sinacalia H. Rob. & Brettell; Sinclairia Hook. & Arn.; Sinoleontopodium Y. L. Chen; Sinosenecio B. Nord.; Smallanthus Mack.; Soaresia Sch.Bip.; Solanecio (Sch.Bip.) Walp.; Solenogyne Cass.; Solidago L.—goldenrod; Soliva Ruiz & Pay.—burrweed; Sommerfeltia Less.; Sonchus L.—sow thistle, sowthistle; Sondottia P. S. Short; Soroseris Stebbins; Spaniopappus B. L. Rob.; Sphaeranthus L.; Sphaereupatorium (O. Hoffm.) Kuntze ex B. L. Rob.; Sphaeroclinium (DC.) Sch.Bip.; Sphaeromeria Nutt.—chickensage; Sphaeromorphaea DC.; Sphagneticola O. Hoffm.—“creeping-oxeye”; Spilanthes Jacq.—toothache flower; Spiracantha Kunth—dogwoodleaf; Spiroseris Rech.f.; Squamopappus Jansen, Harriman & Urbatsch; Stachycephalum Sch.Bip. ex Benth.; Staehefina L.; Standleyanthus R. M. King & H. Rob.; Stanfieldia Small; Staurochlamys Baker; Stebbinsoseris K. L. Chambers—silverpuffs; Steiractinia S. F. Blake; Steirodiscus Less.; Stenachaenium Benth.; Stenocarpha S. F. Blake; Stenocline DC.; Stenopadus S. F. Blake; Stenophalium Anderb.; Stenops B. Nord.; Stenotus Nutt.—mock goldenweed; Stephanochilus Coss. & Durieu ex Maire; Stephanodoria Greene; Stephanomeria Nutt.—wire lettuce; Steptorhamphus Bunge; Stevie Cay.—candyleaf; Steviopsis R. M. King & H. Rob.; Steyermarkina R. M. King & H. Rob.; Stifftia J. C. Mikan; Stilpnogyne DC.; Stilpnolepis Krasch.; Stilpnopappus Mart. ex DC.; Stoebe L.; Stokesia L′Her.; Stomatanthes R. M. King & H. Rob.; Stomatochaeta (S. F. Blake) Maguire & Wurdack; Stramentopappus H. Rob. & V. A. Funk; Streptoglossa Steetz ex F.Muell.; Strotheria B. L. Turner; Struchium P.Browne; Stuartina Sond.; Stuckertiella Beauverd; Stuessya B. L. Turner & F. G. Davies; Stylocline Nutt.—neststraw, woolly fishhooks; Stylotrichium Mattf.; Sventenia Font Quer; Symphyliocarpus Maxim.; Symphyopappus Turcz.; Symphyotrichum—aster (?); Syncalathium Lipsch.; Syncarpha DC.; Syncephalum DC.; Syncretocarpus S. F. Blake; Synedrella Gaertn.; Synedrellopsis Hieron. & Kuntze; Syneilesis Maxim.; Synosma (synonym of Hasteola Raf.); Synotis (C. B. Clarke) C. Jeffrey & Y. L. Chen; Syntrichopappus A. Gray—Fremont's gold; Synurus Iljin; Syreitschikovia Pavlov; Tagetes L.—marigold; Tamananthus V. M. Badillo; Tamania Cuatrec.; Tamaulipa R. M. King & H. Rob.—boneset; Tanacetum L.—tansy, feverfew; Taplinia Lander; Taraxacum Weber ex F. H. Wigg.—dandelion; Tarchonanthus L.; Tarlmounia H. Rob., S. C. Keeley, Skvarla & R. Chan; Teixeiranthus R. M. King & H. Rob.; Telanthophora H. Rob. & Brettell; Telekia Baumg.; Telmatophila Mart. ex Baker; Tenrhynea Hilliard & B. L. Burtt; Tephroseris (Rchb.) Rchb.—fleawort; Tessaria Ruiz & Pav.; Tetrachyron Schltdl.; Tetradymia DC—horsebrush; Tetragonotheca L.—nerveray; Tetramolopium Ness; Tetraneuris Greene—four-nerve daisy; Tetranthus Sw.; Tetraperone Urb.; Thaminophyllum Harv.; Thamnoseris F. Phil.; Thelesperma Less.—greenthread; Thespidium F. Muell. ex Benth.; Thespis DC.; Thevenotia DC.; Thiseltonia Hemsl; Thurovia (synonym of Gutierrezia Lag.); Thymophylla Lag.—pricklyleaf; Thymopsis Benth.; Tiarocarpus Rech.f.; Tietkensia P. S. Short; Tithonia Desf. ex Juss.; Tolpis Adans.—umbrella milkwort; Tonestus A. Nelson—serpentweed; Tourneuxia Coss.; Townsendia Hook.—Townsend daisy; Toxanthes Turcz.; Toiyabea; Tracyina S. F. Blake—Indian headdress; Tragopogon L.—goat's beard, salsify; Traversia Hook.f.; Trichanthemis Regel & Schmalh.; Trichanthodium Sond. & F. Muell.; Trichocline Cass.; Trichocoronis A. Gray—bugheal; Trichocoryne S. F. Blake; Trichogonia (DC.) Gardner; Trichogoniopsis R. M. King & H. Rob.; Trichogyne Less.; Tricholepis DC.; Trichoptilium A. Gray; Trichospira Kunth; Tridactylina (DC.) Sch.Bip.; Tridax L.; Trigonospermum Less.; Trilisa (Cass.) Cass.; Trimorpha—boreal daisy (?); Trioncinia (F. Muell.) Veldkamp; Tripleurospermum Sch.Bip.—mayweed; Triplocephalum O. Hoffm.; Tripolium—sea aster (?); Tripteris; Triptilion Ruiz & Pay.; Triptilodiscus Turcz.; Triniteurybia; Trixis P. Browne—American threefold, threefold, American trixis, California trixis; Troglophyton Hilliard & B. L. Burtt; Tuberostylis Steetz; Tugarinovia /trljin; Turaniphytum Poljakov; Tussilago L.—coltsfoot; Tuxtla Villasenor & Strother; Tyleropappus Greenm.; Tyrimnus (Cass.) Cass.; Uechtritzia Freyn; Ugamia Pavlov; Uleophytum Hieron.; Unxia L.f.; Urbananthus R. M. King & H. Rob.; Urbinella Greenm.; Urmenetea Phil.; Urolepis (DC.) R. M. King & H. Rob.; Uropappus Nutt.—silverpuffs; Urospermum Scop.; Ursinia Gaertn.; Vanclevea Greene; Vanillosmopsis Sch.Bip.; Varilla A. Gray; Varthemia DC.; Vellereophyton Hilliard & B. L. Burtt; Venegasia DC; Venidium (synonym of Arctotis L.); Verbesina L.—crownbeard; Vennonia Schreb—“ironweed (plant)”; Vernoniopsis Humbert; Vieraea Sch.Bip.; Viereckia R. M. King & H. Rob.; Vigethia W. A. Weber; Viguiera Kunth—goldeneye; Vilobia Strother; Virgulaster Semple=Aster L.; Vittadinia A. Rich.; Vittetia R. M. King & H. Rob.; Volutaria Cass.; Wagenitzia Dostal; Waitzia J. C. Wendl.; Wamalchitamia Strother; Warionia Benth. & Coss.; Wedelia Jacq.—“creeping-oxeye”; Willemetia; Welwitschiella O. Hoffm.; Werneria Kunth; Westoniella Cuatrec.; Whitneya A. Gray; Wilkesia A. Gray—iliau; Wollastonia DC. ex Decne.; Wulffia Neck. ex Cass.; Wunderlichia Riedel ex Benth.; Wyethia Nutt.—mule-ears; Xanthisma DC—sleepydaisy; Xanthium L.—cocklebur; Xanthocephalum Willd.; Xanthopappus C. Winkl.; Xeranthemum L.; Xerolekia Anderb.; Xerxes J. R. Grant; Xylanthemum Tzvelev on ; will Xylorhiza—woody aster; Xylothamia G. L. Nesom, Y. B. Suh, D. R. Morgan & B. B. Simpson; Yermo Dorn—desert yellowhead; Youngia Cass.; Zaluzania Pers.; Zandera D. L. Schulz; Zexmenia La Llave; Zinnia L.; Zoegea L.; Zyzyxia Strother.

FAMILY: CANNABACEAE; GENERA: Aphananthe Planchon (syn. Mirandaceltis Sharp); Cannabis L.—Hemp; Celtis L. (syn. Sparrea Hunz. & Dottori); Gironniera Gaudich. (syn. Helminthospermum Thwaites, Nematostigma Planchon); Humulus L. (syn. Humulopsis Grudz.)—Hop; Lozanella Greenman; Parasponia Miguel; Pteroceltis Maxim.; Trema Loureiro (syn. Sponia Decaisne)

FAMILY: RUTACEAE; GENERA: Achuaria Gereau; Acmadenia Bartl. & H. L. Wendl.; Acradenia Kippist; Acronychia J. R. Forst. & G. Forst—Lemon Aspen, et al.; Adenandra Willd.; Adiscanthus Ducke; Aegle Correa—Bael; Aeglopsis Swingle; Afraegle (Swingle) Engl.; Agathosma Willd.; Almeidea A. St.-Hil.; Amyris P. Browne—West Indian Sandalwood; Angostura Roem. & Schult.; Apocaulon R. S. Cowan; Araliopsis Engl.; Asterolasia F. Muell.; Atalantia Correa; Balfourodendron Corr. Mello ex Oliv.; Balsamocitrus Stapf; Boenninghausenia Rchb. ex Meisn.; Boninia Planch.; Boronella Baill.; Boronia Sm.; Bosistoa F. Muell—Bonewoods; Bouchardatia Baill.; Brombya F. Muell.; Burkillanthus Swingle; Calodendrum Thunb.; Casimiroa La Llave; Chloroxylon DC Ceylon Satinwood; Choisya Kunth—Mexican orange; Chorilaena Endl.; Citropsis (Engl.) Swingle & M. Kellerm—African orange cherry; Citrus L.—Citrus; Clausena Burm.f.; Clymenia Swingle; Cneoridium Hook.f.; Cneorum L. (formerly in Cneoraceae); Coleonema Bartl. & H. L. Wendl.—Breath of Heaven; Comptonella Baker f.; Coombea P. Royen; Correa Andrews; Crowea Sm.; Cyanothamnus Lindl.; Decagonocarpus Engl.; Decatropis Hook.f.; Decazyx Pittier & S. F. Blake; Dendrosma Pancher & Sebert; Dictamnus L.—Burning-bush; Dictyoloma A.Juss.; Diosma L.; Diphasia Pierre; Diphasiopsis Mendonca; Diplolaena R. Br.; Drummondita Harv.; Dutaillyea Baill.; Echinocitrus Tanaka; Empleuridium Sond. & Harv.; Empleurum Aiton; Eremocitrus Swingle; Eriostemon Sm.; Erythrochiton Nees & Mart.; Esenbeckia Kunth; Euchaetis Bartl. & H. L. Wendl.; Euodia J. R. Forst. & G. Forst.; Euxylophora Huber; Evodiella Linden; Fagaropsis Mildbr. ex Siebenl.; Feroniella Swingle; Flindersia RBr. Crow Ash, Cudgerie; Fortunella Swingle—Kumquat; Galipea Aubl.; Geijera Schott—Wilga, Axebreakers; Geleznowia Turcz.; Glycosmis Correa; Halfordia F. Muell.; Haplophyllum A.Juss.; Helietta Tul.; Hortia Vand.; Ivodea Capuron; Kodalyodendron Borhidi & Acuna; Leionema (F. Muell.) Paul G.Wilson.; Leptothyrsa Hook.f.; Limnocitrus Swingle; Limonia L.; Lubaria Pittier; Lunasia Blanco; Luvunga Buch.-Ham. ex Wight & Arn.; Maclurodendron T. G. Hartley; Macrostylis Bartl. & H. L. Wendl.; Medicosma Hook.f.; Megastigma Hook.f.; Melicope J. R. Forst. & G. Forst—Corkwood, Alani; Merope M. Roem.; Merrillia Swingle; Metrodorea A. St.-Hil.; Microcitrus Swingle; Microcybe Turcz.; Micromelum Blume; Monanthocitrus Tanaka; Monnieria Loefl.; Muiriantha C. A. Gardner; Murraya L.—Curry tree; Myrtopsis Engl.; Naringi Adans.; Naudinia Planch. & Linden; Nematolepis Turcz.; Neobyrnesia J. A. Armstr.; Neoschmidia T. G. Hartley, gen. nov.; Nycticalanthus Ducke; Oricia Pierre; Oriciopsis Engl.; Orixa Thunb.; Oxanthera Montrouz.; Pamburus Swingle; Paramignya Wight; Peltostigma Walp.; Pentaceras Hook.f.; Phebalium Vent.; Phellodendron Rupr.—Cork-tree; Philotheca Rudge; Phyllosma Bolus; Pilocarpus Vahl; Pitavia Molina; Pitaviaster T. G. Hartley; Platydesma H.Mann; Pleiospermium (Engl.) Swingle; Plethadenia Urb.; Polyaster Hook.f.; Poncirus Raf.—Trifoliate orange; Psilopeganum Hemsl.; Ptelea L.—Hoptree; Raputia Aubl.; Rauia Nees & Mart.; Raulinoa R. S. Cowan; Ravenia Veil.; Raveniopsis Gleason; Rhadinothamnus Paul G.Wilson; Ruta L.—Rue; Rutaneblina Steyerm. & Luteyn; Sarcomelicope Engl.; Severinia Ten.; Sheilanthera I. Williams; Skimmia Thunb.—Skimmia; Spathelia L.; Spiranthera A. St.-Hil.; Stauranthus Liebm.; Swinglea Merr.; Teclea Delile; Tetractomia Hook.f.; Tetradium Lour.—Euodia; Thamnosma Torr. & Frem.; Ticorea Aubl.; Toddalia Juss.; Toddaliopsis Engl.; Tractocopevodia Raizada & V.Naray.; Triphasia Lour.; Urocarpus J. Drumm. ex Harv.; Vepris Comm. ex A.Juss.; Wenzelia Merr.; Zanthoxylum L.—Toothache tree; Zieria Sm.

FAMILY: ROSACEAE; GENERA: Acaena; Acomastylis; Adenostoma; Agrimonia; Amelanchier; ×Amelasorbus; Amygdalophora; Amygdalopsis; Amygdalus; Aphanes; Aria; Argentina; ×Ariosorbus; Aronia; Aruncus; Atomostigma; Batidaea; Bencomia; Brachycaulos; Brayera; Cerapadus; Ceraseidos; Cerasus; Cercocarpus; Chamaebatiaria; Chamaemeles; Chaenomeles; Chamaemespilus; Chaemaerhodos; Cliffortia; Coleogyne; Coluria; Comarella; Comarobatia; Comaropsis; Comarum; Cormus; Cotoneaster; Cowania; +Crataegomespilus; Crataegus;×Crataemespilus; Cydonia; Dalibarda; Dasiphora; Dendriopoterium; Dichotomanthes; Docynia; Dryadanthe; Dryas; Eriobotrya; Eriogynia; Eriolobus; Erythrocoma; Exochorda; Fallugia; Farinopsis; Filipendula; Fragaria; Geum; Gillenia; Hagenia; Hesperomeles; Heteromeles; Holodiscus; Horkelia; Horkeliella; Hulthemia (synonym of Rosa);×Hulthemosa (Hulthemia×Rosa); Ivesia; Kageneckia; Kerria; Lachemilla; Laurocerasus; Leucosidea; Lindleya; Luetkea; Lyonothamnus; Maddenia; Malacomeles; ×Malosorbus; Malus; Marcetella; Mespilus; Micromeles; Nagelia; Neillia; Neviusia; Novoseiversia; Nutallia; Oemleria; Oncostylus; Oreogeum; Orthurus; Osteomeles; Padellus; Parageum; Pentactina; Pentaphylloides; Peraphyllum; Persica; Petrophyton; Photinia; Physocarpus; Polylepis; Porteranthus; Potentilla; Poteridium; Poterium; Pourthiaea; Prinsepia; Prunus; Pseudocydonia; Purshia; Pyracantha; Pygeum; ×Pyronia; Pyrus; ×Rhaphiobotyra; Rhaphiolepis; Rhodotypos; Rosa; Rub us; Sanguisorba; Sarcopoterium; Sibbaldia; Sibbaldiopsis; Sibiraea; Sorbaria; ×Sorbaronia; ×Sorbocotoneaster; ×Sorbopyrus; Sorbus; Spenceria; Spiraea; Stephanandra; Taihangia; Tetraglochin; Torminalia; Trichothalamus; Tylosperma; Ulmaria; Vauquelinia; Waldsteinia; Xerospiraea; Zygalchemilla.

FAMILY: LAMIACEAE; GENERA: Acanthomintha; Achyrospermum; Acinos; Acrocephalus; Acrotome; Acrymia; Adelosa; Aegiphila; Aeollanthus; Agastache; Ajuga; Ajugoides; Alajja; Alvesia; Amasonia; Amethystea; Anisochilus; Anisomeles; Archboldia; Asterohyptis; Ballota; Basilicum; Becium; Benguellia; Blephilia; Bostrychanthera; Bovonia; Brachysola; Brazoria; Bystropogon; Calamintha; Callicarpa; Capitanopsis; Capitanya; Caryopteris; Catoferia; Cedronella; Ceratanthus; Chaiturus; Chamaesphacos; Chaunostoma; Chelonopsis; Chloanthes; Cleonia; Clerodendrum; Clinopodium; Colebrookea; Collinsonia; Colquhounia; Comanthosphace; Congea; Conradina; Coridothymus; Cornutia; Craniotome; Cryphia; Cuminia; Cunila; Cyanostegia; Cyclotrichium; Cymaria; Dauphinea; Dicerandra; Dicrastylis; Discretitheca; Dorystoechas; Dracocephalum; Drepanocaryum; Elsholtzia; Endostemon; Englerastrum; Eremostachys; Eriope; Eriophyton; Eriopidion; Eriothymus; Erythrochlamys; Euhesperida; Eurysolen; Faradaya; Fuerstia; Galeopsis; Garrettia; Geniosporum; Glechoma; Glechon; Glossocarya; Gmelina; Gomphostemma; Gontscharovia; Hanceola; Haplostachys; Haumaniastrum; Hedeoma; Hemiandra; Hemigenia; Hemiphora; Hemizygia; Hesperozygis; Heterolamium; Hoehnea; Holmskioldia; Holocheila; Holostylon; Horminum; Hosea; Hoslundia; Huxleya; Hymenocrater; Hymenopyramis; Hypenia; Hypogomphia; Hyptidendron; Hyptis; Hyssopus; Isodictyophorus; Isodon; Isoleucas; +Kalaharia; Karomia; Keiskea; Killickia; Kudrjaschevia; Kurzamra; Lachnostachys; Lagochilus; Lagopsis; Lallemantia; Lamiophlomis; Lamium; Lavandula; Leocus; Leonotis; Leonurus; Lepechinia; Leucas; Leucophae; Leucosceptrum; Limniboza; Lophanthus; Loxocalyx; Lycopus; Macbridea; Madlabium; Mallophora; Marmoritis; Marrubium; Marsypianthes; Matsumurella; Meehania; Melissa; Melittis; Mentha; Meriandra; Mesona; Metastachydium; Microcorys; Micromeria; Microtoena; Minthostachys; Moluccella; Monarda; Monardella; Monochilus; Mosla; Neoeplingia; Neohyptis; Neorapinia; Nepeta; Newcastelia; Nosema; Notochaete; Obtegomeria; Ocimum; Octomeron; Ombrocharis; Oncinocalyx; Origanum; Orthosiphon; Otostegia; +Ovieda; Oxera; Panzerina; Paralamium; Paraphlomis; Paravitex; Peltodon; Pentapleura; Perilla; Perillula; Peronema; Perovskia; Perrierastrum; Petitia; Petraeovitex; Phlomidoschema; Phlomis; Phyllostegia; Physopsis; Physostegia; Piloblephis; Pitardia; Pityrodia; Platostoma; Plectranthus; Pogogyne; Pogostemon; Poliomintha; Prasium; Premna; Prostanthera; Prunella; Pseudocarpidium; Pseudocaryopteris; Pseudoeremostachys; Pseudomarrubium; Puntia; Pycnanthemum; Pycnostachys; Rabdosiella; Renschia; Rhabdocaulon; Rhaphiodon; Rhododon; Rosmarinus; Rostrinucula; Rotheca; Roylea; Rubiteucris; +Rydingia; Sabaudia; Saccocalyx; Salazaria; Salvia; Satureja; Schizonepeta; Schnabelia; Scutellaria; Sideritis; Siphocranion; Solenostemon; Spartothamnella; Sphenodesme; Stachydeoma; Stachyopsis; Stachys; Stenogyne; Sulaimania; Suzukia; Symphorema; Symphostemon; Synandra; Syncolostemon; Tectona; Teijsmanniodendron; +Tetraclea; Tetradenia; Teucridium; Teucrium; Thorncroftia; Thuspeinanta; Thymbra; Thymus; Tinnea; Trichosterna; Tripora; Tsoongia; Vitex; Viticipremna; +Volkameria; Warnockia; Wenchengia; Westringia; Wiedemannia; Wrixonia; Xenopoma; Zataria; Zhumeria; Ziziphora.

For example, in addition to members of the group Cannanbaceae, the example embodiments may be used with members of the group Solanaceae, which include annually-grown herbaceous plants, such as, Nicotiana tabacum, or cultivated tobacco, which is found only in cultivation, and is considered the most commonly grown of all plants in the Nicotiana genus, and whose leaves are commercially grown in many countries to be processed into tobacco. In addition, other members of the group Solanacea include wild Nicotiana species, such as Nicotiana sylvestris, Nicotiana tomentosiformis, Nicotiana otophora, etc.

The example embodiments described herein will be further illustrated in the following, non-limiting Examples. The Examples are illustrative of various embodiments only and do not limit the claimed invention regarding the materials, conditions, weight ratios, process parameters and the like recited herein.

### EXAMPLE 1

A comparison of extracts prepared according to the example embodiments described herein and other solvent systems was carried out.

Extract samples were prepared by weighing out a 7.0 gram aliquot of plant material (i.e., whole dried cannabis flowers) and removing all stems by hand. The flowers were separated and homogenized by hand into smaller pieces to form particles with a diameter in the range of about 0.5 mm to 3 mm. A desired solvent was added to a vessel such that about a 10:1 mass ratio of desired solvent to plant material will be achieved and this solvent was then cooled to a predetermined temperature (see Table 1, Sample Nos. 1-16) either by direct addition of dry ice to the solvent mixture or through the use of an external dry ice and acetone cooling bath in which the vessel was placed. Each homogenized sample was then added to the solvent in the vessel and allowed to be extracted by incubating for about 10 minutes with mixing on a magnetic stir plate. The plant material was then rapidly filtered through a metal mesh strainer to remove larger particles from the solvent. The extracted plant material was compressed with a spatula against the surface of the strainer to remove remaining solvent absorbed by the plant matter. A second filtration was then carried out under vacuum using a Whatman Grade 1 Filter paper to remove fine particles of 11 micron (μm) or larger. The solvent was then removed from each sample by rotary evaporation.

TABLE 1

| Sample ID No. | Solvent System/Conditions |
|---|---|
| 1 | Acetone, CO₂at about −78.5° C. |
| 2 | Acetone, bathat about −78.5° C. |
| 3 | Acetone, bath, 0.0° C. |
| 4 | Ethanol, CO₂at about −78.5° C. |
| 5 | Ethanol, bathat about −78.5° C. |
| 6 | Diethyl Ether, CO₂at about −78.5° C. |
| 7 | Diethyl Ether, bathat about −78.5° C. |
| 8 | MEK, CO₂at about −78.5° C. |
| 9 | MEK, bathat about −78.5° C. |
| 10 | Ethyl Lactate, bath, −10.0° C. |
| 11 | Butyl Acetate, CO₂, −60.0° C. |
| 12 | MTBE, CO₂at about −78.5° C. |
| 13 | MTBE, bathat about −78.5° C. |
| 14 | Ethyl Acetate, CO₂at about −78.5° C. |
| 15 | Ethyl Acetate, bathat about −78.5° C. |
| 16 | Pentane, CO₂at about −78.5° C. |
| 17 | Butane Cylinder, −17.4° C. |

For Sample ID No. 17, a butane extraction was carried out using a conventional butane honey oil (BHO) 30 mm extractor cylinder. A 7.0 gram aliquot of whole dried cannabis flowers was measured and all stems were removed by hand. The flowers were separated and homogenized into moderate uniform pieces known as ‘popcorn buds’ typically used in butane extraction with an average particle size diameter in the range of 5 mm to 8 mm. The entire dried cannabis flower sample was then placed in the BHO extractor cylinder, which was then assembled, held upright such that the perforated portion was facing down, then injected with approximately 150 g of butane through into the top of the cylinder. The extract and butane mixture was captured in a shallow 1L pyrex beaker. The beaker was then placed in a room temperature (20-24° C.) water bath in a fume hood until all solvent had evaporated. The resulting cannabis extract was then resuspended in 60 ml of HPLC grade liquid pentane, filtered under vacuum through a Whatman grade 1 filter paper to remove particles 11 μm in size or larger. The 1L pyrex beaker was then rinsed with an additional 25 ml of pentane and also filtered. The extract was then subjected rotary evaporation to remove solvent and collected.

### EXAMPLE 2

After solvent removal, all extract resin samples collected were resuspended in 50 ml of anhydrous ethanol for further analysis. Cannabinoid content in each sample was determined using Agilent 1100 series HPLC/PDA with Luna 5μ C18 column 100A. Terpene content was determined in each sample using Agilent 6890 GC/5973MSD with RXI-35SilMS 30m×0.25 mm ID×0.25 μm d.f.

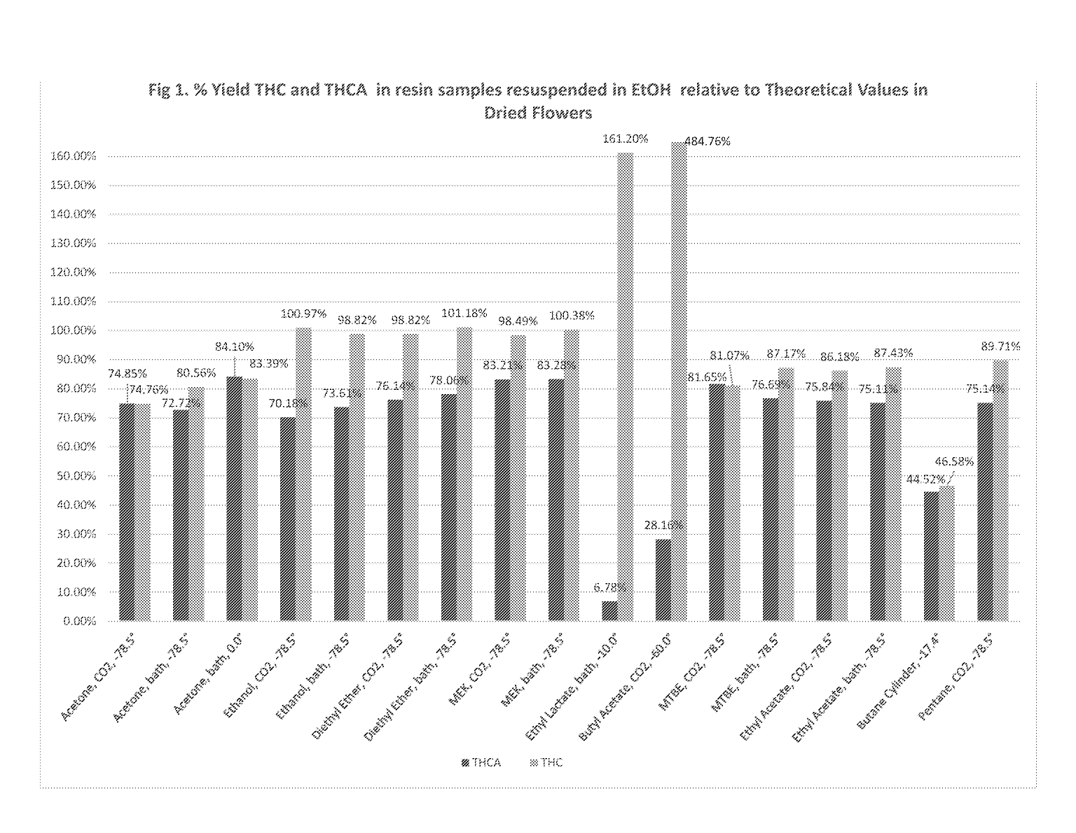

The data in FIG. 1 show what the yield of THCA and THC extraction obtained compared to a relative 100% yield, as determined by the 9:1 Chloroform:Methanol validated extraction method. FIG. 1 also exemplifies some of the issues known and considered unfavorable with high boiling-point solvents. For instance, because of the high heat and low pressure conditions required to evaporate ethyl lactate and butyl acetate, these conditions provide sufficient thermal energy to convert THCA into THC, thereby altering the natural profile found in the starting cannabis flowers. Yields of 161% and 484% THC when using ethyl lactate and butyl acetate, respectively, provide evidence of the thermally driven conversion of THCA to THC. FIG. 1 further shows that THC yields were nearly equivalent to THCA yields when using our acetone solvent systems/methods for extraction, indicating that the natural ratio of THC and THCA in the cannabis resins produced by our acetone extraction system/method are unaltered by their differences in solubility in acetone.

Since THCA is found in much greater quantity compared to THC in the cannabis flowers, these data are important because creating a whole plant extract of cannabis that contains the same relative amounts of cannabinoids and terpenes without undesirable constituents (i.e., a representative profile of such desirable constituents in amounts or ratios similar to what is found in the starting input botanical material) is ideal for the solvent to be optimized for the greatest solubility of THCA. Cannabis extracts produced with the example embodiments (acetone system) contain a greater yield of THCA from the dried flowers when compared to common commercial extraction techniques (e.g., cold Ethanol, Butane, etc.) and these results demonstrate that the example embodiments are among the highest yielding solvents tested.

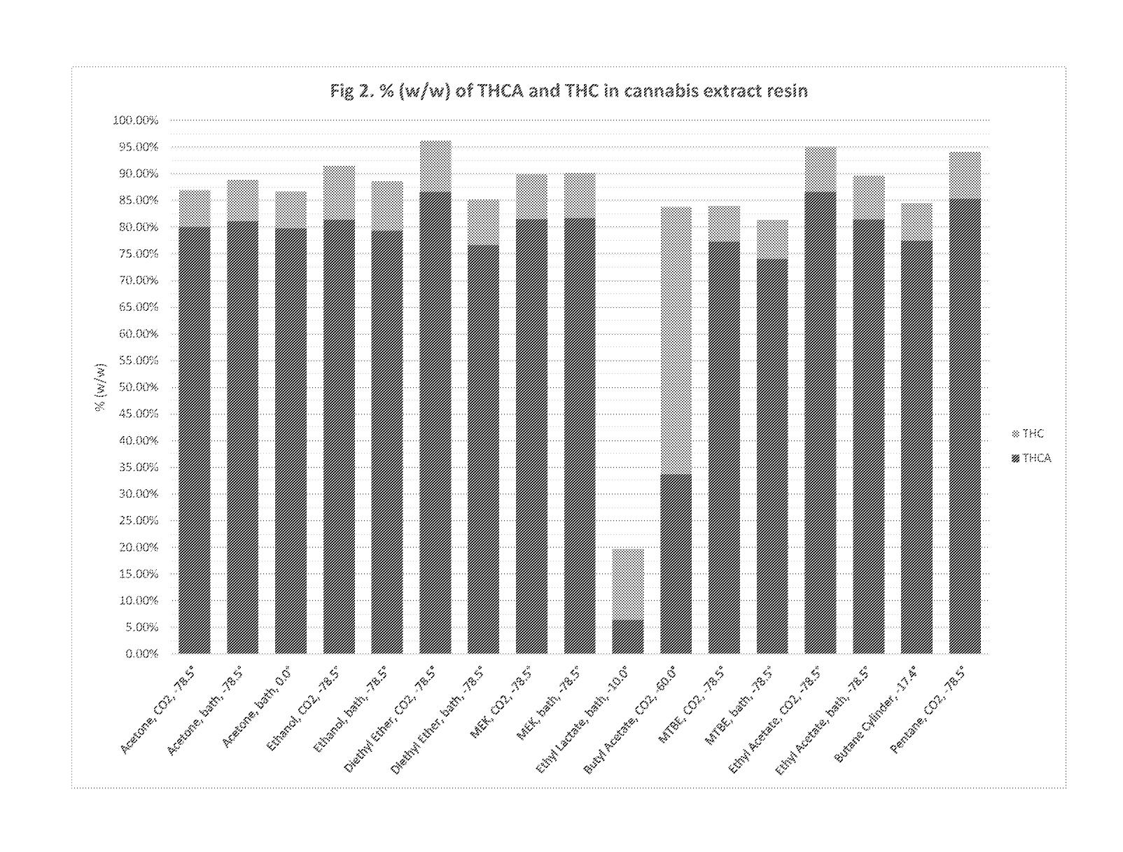

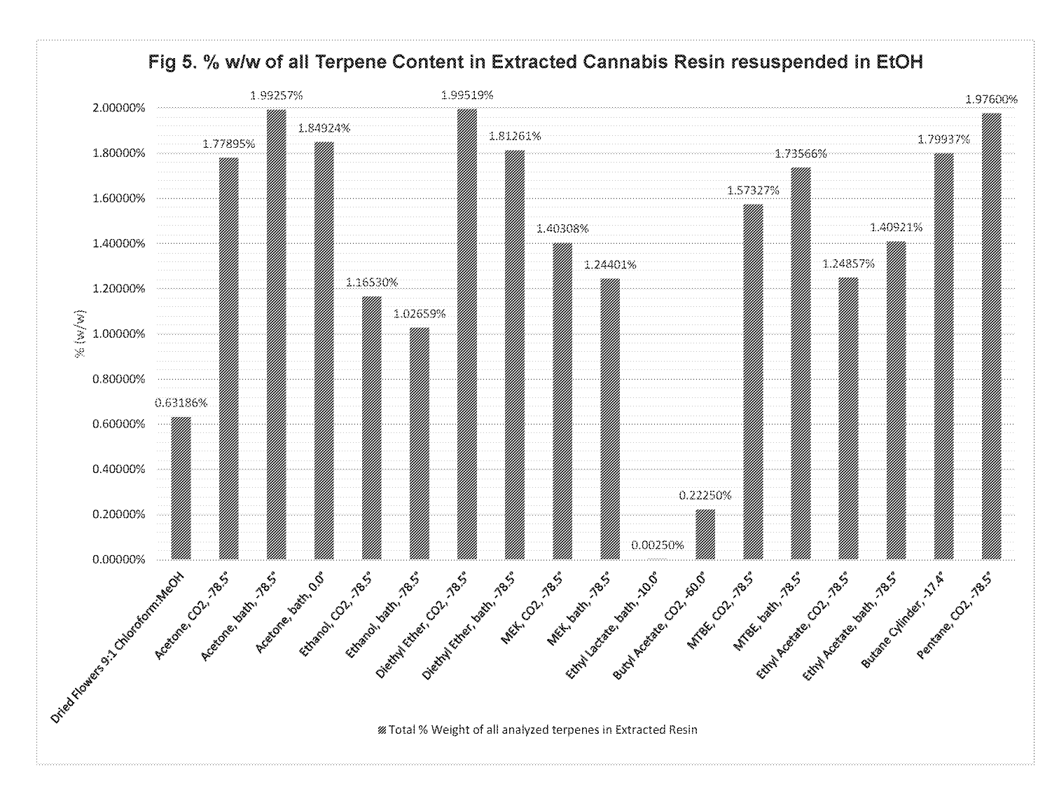

The data in FIG. 2 show % w/w of THCA and THC in Cannabis extract resin. Of all the Samples, cannabis extract produced using diethyl ether as the solvent appeared to contain the highest concentration of cannabinoids (as exemplified by THCA and THC 96.27% w/w overall). FIG. 5 also shows that diethyl ether produces the greatest yield of terpenes with 1.995% w/w overall. Diethyl ether is a non-polar solvent and as such would be expected to produce a high yield of non-polar cannabinoid and terpene compounds.

Acetone and ethanol, both polar solvents, are expected to produce a lower yield because of their chemical nature. However, FIG. 5 shows that despite this polar nature shared by acetone and ethanol, under certain conditions acetone (i.e. acetone only at about −78.5° C.) is capable of extracting nearly equivalent amounts of terpenes as expected from non-polar solvents such as pentane and diethyl ether.

All acetone solvent systems tested are comparable (and in some cases equal in ability) to extract the non-polar terpene components relative to non-polar solvents such as pentane, butane and diethyl ether.

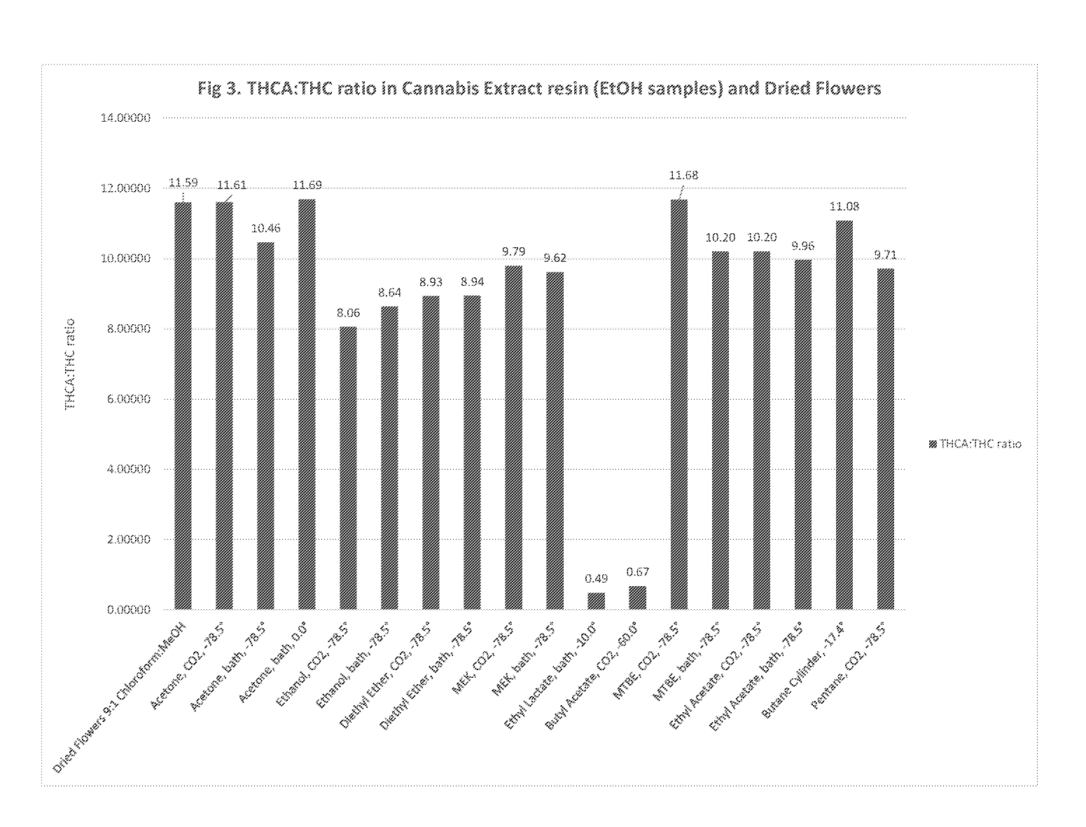

FIG. 3 shows THCA:THC ratio in cannabis extract resin and Dried Flowers. The ratio of THCA to THC contained in the the first column of this figure (Dried Flowers') represents the natural ratio (11.59) of THCA:THC found in the dried cannabis flowers as determined by 9:1 chloroform extraction and validated HPLC analysis. FIG. 3 shows that extracts produced using:

(1) acetone with CO₂ at about −78.5° C.;

(2) acetone at 0° C.; or

(3) Methyl-tert butyl ether (MTBE) at about −78.5° C. produce an extract with this same 11.6 ratio of THCA:THC from the same strain of cannabis. This evidence indicates there are multiple systems that incorporate the use acetone as the extraction solvent to achieve the same natural profile of THCA and THC in a cannabis resin much more accurately than the common commercial extraction methods that use ethanol (8.06, 8.64 THCA:THC) or butane (11.08 THCA:THC) as the solvent.

Although it appears that acetone +CO₂ and acetone only at 0° C. are both equally capable of producing a cannabis extract that maintains the natural THCA:THC ratio, it was determined under further testing that acetone only at 0° C. produces a lower quality extract because of the detectable presence of chlorophyll in a sample prepared under these conditions. In contrast, a cannabis extraction prepared using acetone +CO₂ at about −78.5° C. had no detectable amount of chlorophyll.

Taken together with FIG. 1, FIG. 3 also shows that acetone +CO₂ extraction is able to produce a cannabis extract with a THCA:THC ratio representative of the natural profile found in cannabis flowers at the greatest yield (of cannabinoids and terpenes) compared to all other solvent systems tested.

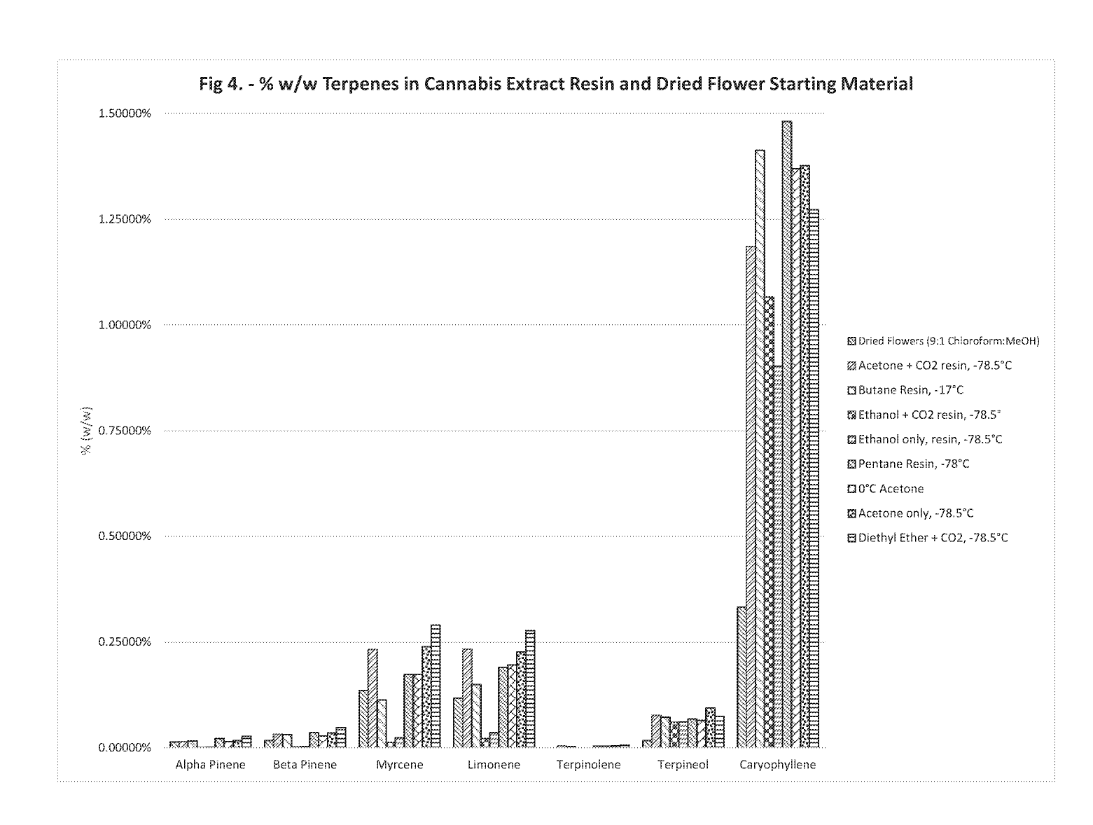

FIG. 4 shows % w/w terpenes in cannabis extract resin and dried flower starting material. In particular, FIG. 4 shows that by producing a cannabis extract resin from dried flowers, all terpene components of the mixture are concentrated, regardless of the solvent used. FIG. 4 also shows that extraction with acetone only (i.e., no CO₂) at about −78.5° C. is an advantageous solvent system for the overall extraction of each individual terpenes analyzed. Pentane appears to be equal to or greater than acetone with respect to the yield of individual terpenes in a cannabis extract. However, the evidence from FIG. 1 showing greater solubility of THC relative to THCA in pentane indicates that the natural THCA:THC ratio is altered when pentane is used to produce a cannabis extract and is therefore less favorable than acetone when a representative whole plant extraction is desired. Additionally, all acetone solvent extractions systems (‘acetone with CO₂ at about −78.5° C.’, ‘acetone only at about −78.5° C.’, and ‘acetone only at 0° C.’) produced a cannabis extract with % w/w terpene yields comparable to butane systems and also consistently greater yields for each terpene compared with cannabis extract produced using either cold ethanol systems tested, but without the undesirable constituents or features typically experienced with such systems.

Evidence from FIGS. 1, 3 and 4 congruently provide evidence that cannabis extractions using acetone under the conditions of the example embodiments contain the best overall combination of cannabinoid fraction and terpene fraction % w/w yields relative to cannabis extracts produced by common commercial extraction methods (e.g., cold ethanol, butane).

FIG. 5 show % w/w of all terpene content in extracted cannabis resin resuspended in EtOH. Although only present in small quantities relative to cannabinoids, terpenes have known biological activity at very low concentrations. Cannabis extracts with relatively higher overall terpene content are considered to be of greater quality. Although FIG. 5 appears to show that diethyl ether produces a cannabis extract with % w/w terpene fraction greater than the best acetone solvent system extraction (i.e., acetone bath, at about −78.5° C.), dietheyl ether's preferential THC solubility as indicated in FIG. 1 and THCA:THC ratio of 8.9 (less than the natural profile indicated by the 9:1 chloroform:methanol extraction, ‘dried flowers’ sample) in FIG. 3 indicates that diethyl ether's overall suitability for use as a whole plant cannabis extraction solvent system is less preferred than either acetone, butane or pentane. This figure also shows that all acetone extractions produce cannabis resins with greater terpene content than cannabis resins produced with ethanol. It is also noted that FIG. 5 is consistent with FIG. 4 because it shows that cold ‘acetone bath −78.5° C.’ solvent extraction is a superior solvent system for extracting the greatest possible quantity of total terpenes from starting cannabis flowers.

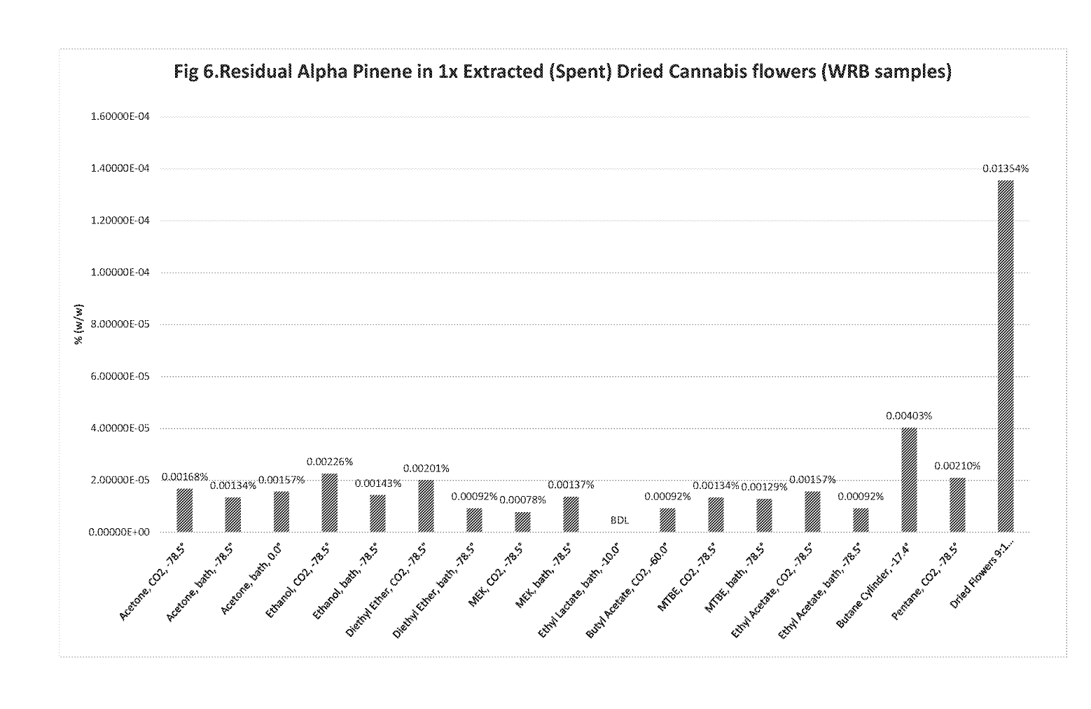

FIGS. 6-12 summarize the analysis results of residual terpene content remaining in the dried cannabis flowers (spent) after one extraction of the input cannabis flowers was carried out. Each dried cannabis sample (including the virgin input cannabis flowers baseline) was extracted via a validated protocol using 9:1 chloroform:methanol solvent system. The far right column (‘Bud from Bag #4’) shows the % w/w of terpenes in the non-extracted input cannabis flowers (i.e. relative 100%). Most preferred results are those which show no residual terpene remaining.

In particular, FIG. 6 shows residual remaining alpha pinene in 1× extracted/spent dried cannabis flowers (i.e., WRB (spent) samples). It will be appreciated that when comparing each individual example embodiment (acetone solvent systems) to the corresponding ethanol solvent system (i.e. ‘Acetone+CO₂at about −78.5° C.’ compared with ‘Ethanol+CO₂at about −78.5° C’; ‘Acetone onlyat about −78.5° C.’ compared with ‘Ethanol onlyat about −78.5° C.’), FIG. 6 shows that a greater amount of residual alpha pinene remains with butane and with pentane relative to all 3 acetone solvent system extractions tested. FIG. 6 also shows that less residual alpha pinene remains in the extracted cannabis flowers when either ‘acetone only at about −78.5° C.’ or ‘acetone+CO₂ at about −78.5° C.’ system is used as the solvent compared to cold ethanol with CO₂ at about −78.5° C.

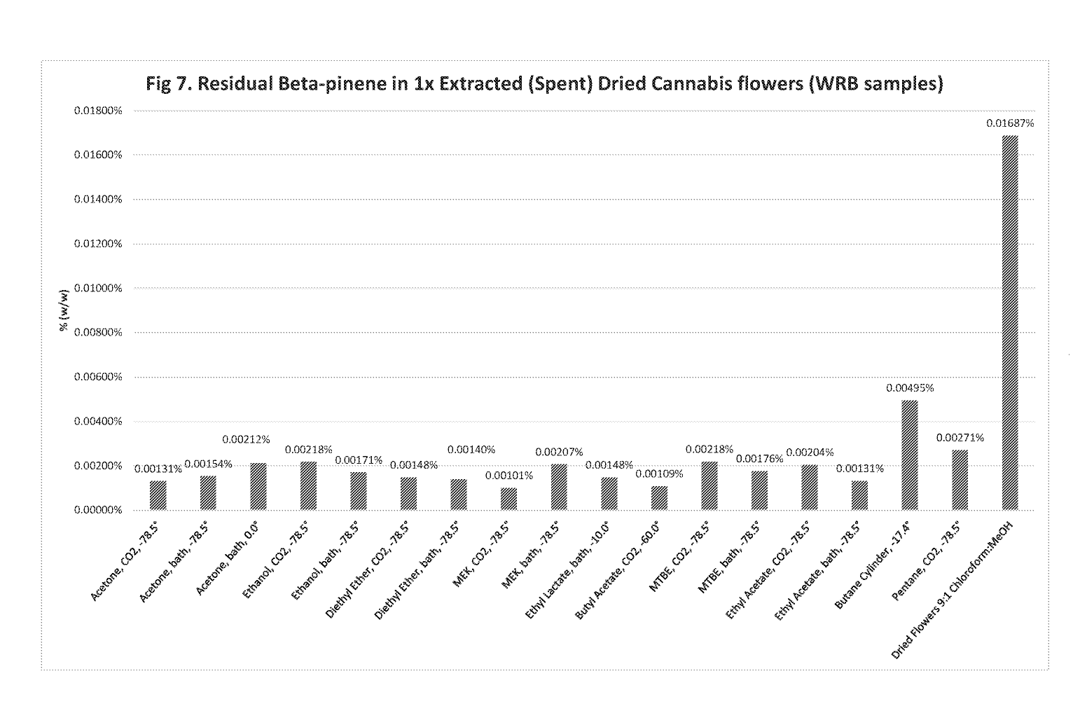

FIG. 7 shows residual beta pinene remaining in 1× extracted (spent) dried cannabis flowers (WRB samples). In particular, FIG. 7 illustrates of all samples of cannabis flowers extracted with: acetone; butane; or ethanol, the ‘acetone+CO₂at about −78.5° C.’ solvent system resulted in the lowest residual beta pinene remaining in the extracted cannabis flower. Butane and pentane appear to be the least efficient at extracting beta-pinene as indicated by the greatest residual beta pinene in extracted cannabis flowers relative to all solvent systems tested.

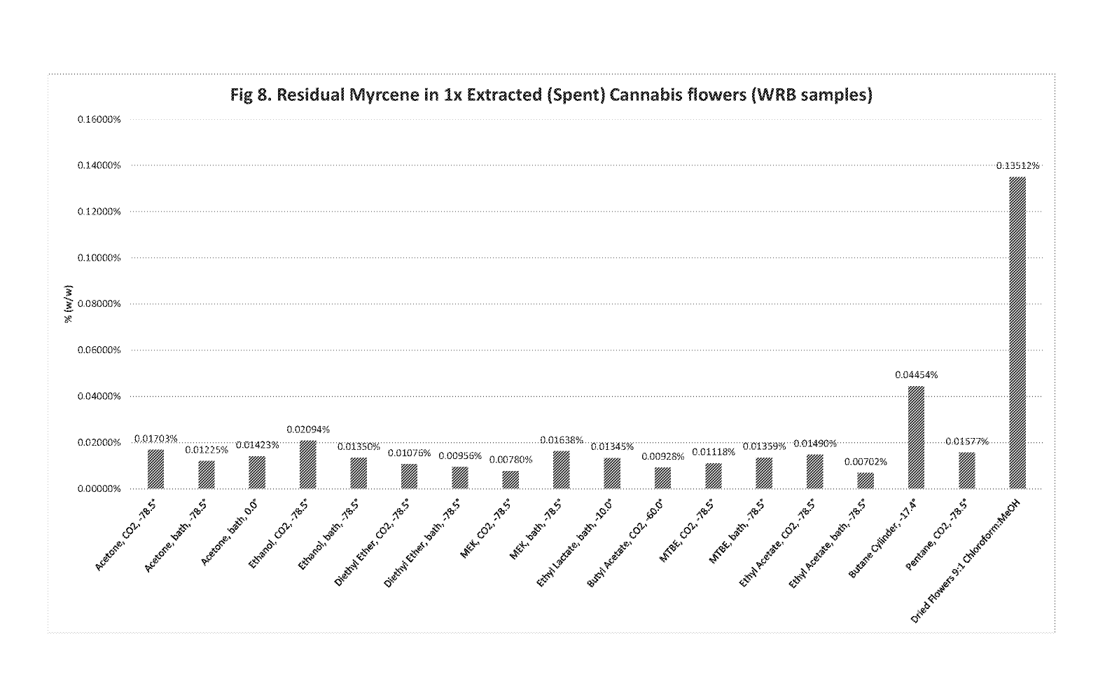

FIG. 8 shows residual myrcene remaining in 1× extracted (spent) dried cannabis flowers (WRB samples). Similar to alpha pinene, a comparison of equivalent acetone and ethanol solvent systems (i.e. ‘Acetone +CO₂at about −78.5° C.’ compared with ‘Ethanol+CO₂at about −78.5° C.’; ‘Acetone onlyat about −78° C.’ compared with ‘Ethanol onlyat about −78.5° C.’), indicating that acetone is more efficient at extracting myrcene relative to ethanol. All acetone systems (example embodiments) are more efficient at extracting myrcene relative to an extraction with butane solvent.

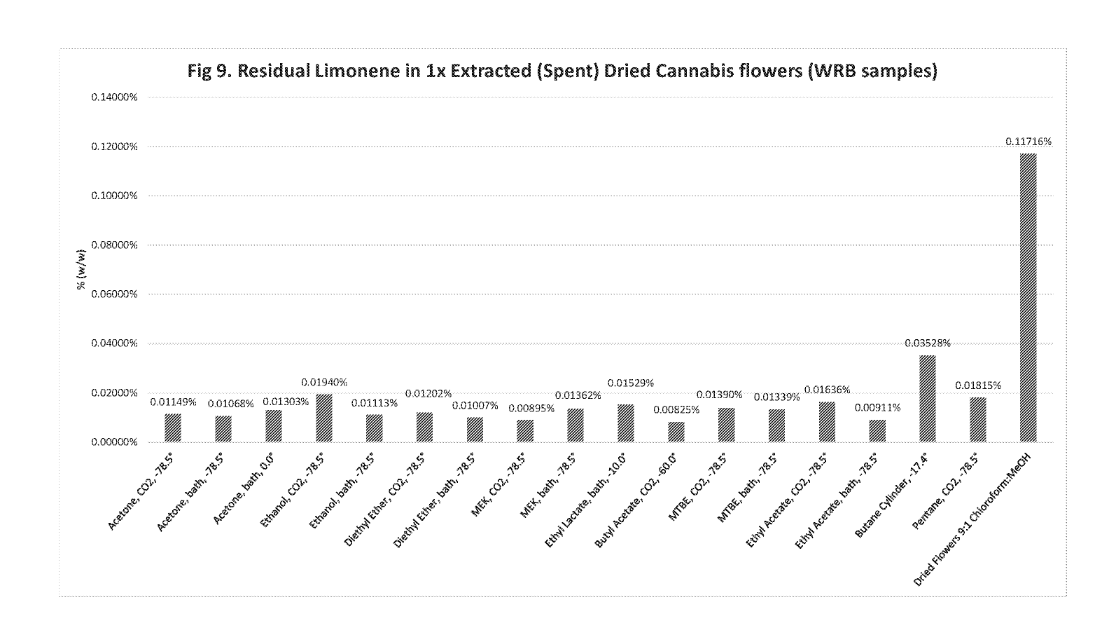

FIG. 9 shows the residual limonene remaiing in 1× Extracted (spent) Dried Cannabis flowers (WRB samples). As can be seen, ‘Ethanol+CO₂at about −78.5° C.’ was less efficient than ‘pentane+CO₂at about −78.5° C.’ with respect to limonene extraction. Butane appears to be the least efficient at extracting limonene relative to all other solvents systems. Equivalent acetone and ethanol solvent systems comparisons (i.e. ‘Acetone+CO₂at about −78.5° C.’ compared with ‘Ethanol+CO₂at about −78.5° C.’; ‘Acetone onlyat about −78° C.’ compared with ‘Ethanol onlyat about −78.5° C.’) show acetone to be more efficient at extracting limonene as indicated by lower relative residual limonene in extracted cannabis flowers.

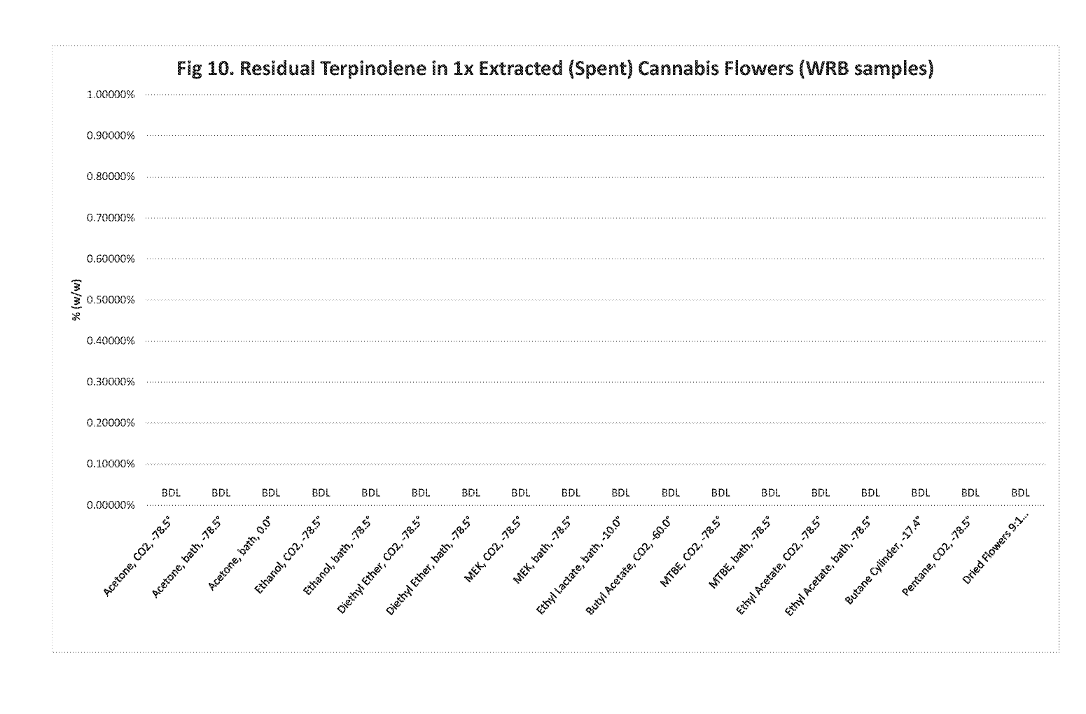

FIG. 10 shows residual terpinolene remaining in 1× extracted dried cannabis flowers (WRB samples). No conclusions can be drawn for Terpinolene extraction efficiency from FIG. 10; no terpinolene was detected in the input cannabis flowers (‘Bud from Bag #4’ i.e. the Relative 100% of any Terpinolene present in unadulterated cannabis flowers) or in the extracted cannabis flowers. It is noted that because of the analytical device's limit of detection, the residual quantity of terpinolene in extracted cannabis flowers after a single extraction cannot be determined. In addition, FIG. 4 shows that all three example embodiment acetone extraction systems and diethyl ether+CO₂ at about −78.5° C. extractions were able to extract and concentrate terpinolene above the lower detection limit of the analytical device, thus indicating all acetone systems and diethyl ether are superior at extracting terpinolene compared with the 9:1 chloroform:methanol extraction system.

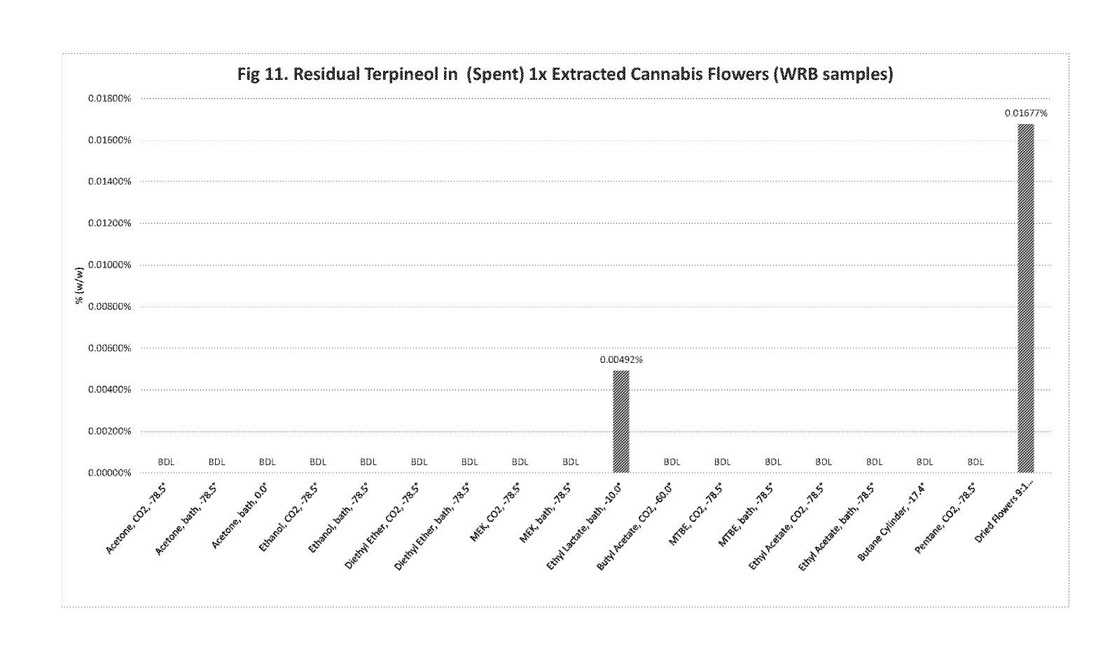

FIG. 11 shows residual terpineol remaining in 1× extracted dried cannabis flowers (WRB samples). The results suggest that all solvents, with the exception of ethyl lactate, are effectively equivalent in efficiency of terpineol extraction.

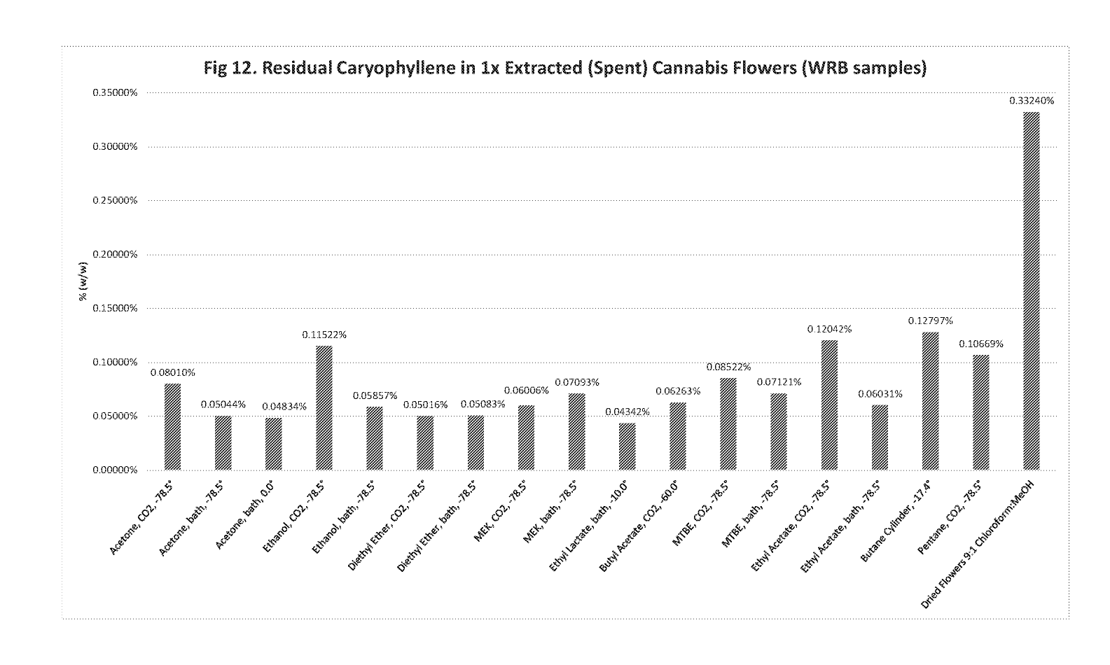

FIG. 12 shows residual caryophyllene remaining in 1× extracted dried cannabis flowers (WRB samples). Equivalent acetone and ethanol extraction solvent system comparisons (i.e. ‘Acetone+CO₂at about −78.5° C.’ compared with ‘Ethanol+CO₂at about −78.5° C.’; ‘Acetone onlyat about −78.5° C.’ compared with ‘Ethanol onlyat about −78.5° C.’), show that acetone is more efficient/effective at extracting caryophyllene as indicated by lower relative residual caryophyllene remaining in extracted cannabis flowers. ‘Acetone onlyat about −78.5° C.’ was more effective than all ethanol, butane and pentane extraction solvent systems. With the exception of terpinolene and terpineol (FIGS. 10 and 11), FIGS. 6-9, and 12 provide evidence that butane is the least efficient at extracting terpenes relative to all other solvent systems tested as indicated by the % w/w of all residual terpenes remaining in extracted cannabis flowers.

### EXAMPLE 3

A comparison of dried virgin (unextracted/unadulterated) cannabis flowers and extracted cannabis flowers was carried out. Dried flowers, extracted (spent) flowers and What Remains Behind (a/k/a “WRB”) samples were prepared by extraction of approximately 100 mg of homogenized and sieved (2 mm screen size) dried cannabis flowers. The ground cannabis flower for each was then mixed with 30 ml of 9:1 chloroform methanol solution at room temperature and sonicated. After incubation, the extraction mixture was centrifuged and the liquid extract decanted into a GC sample vial. The solution was then analyzed by HPLC/FID for cannabinoid contents or GC/MS for terpene content.

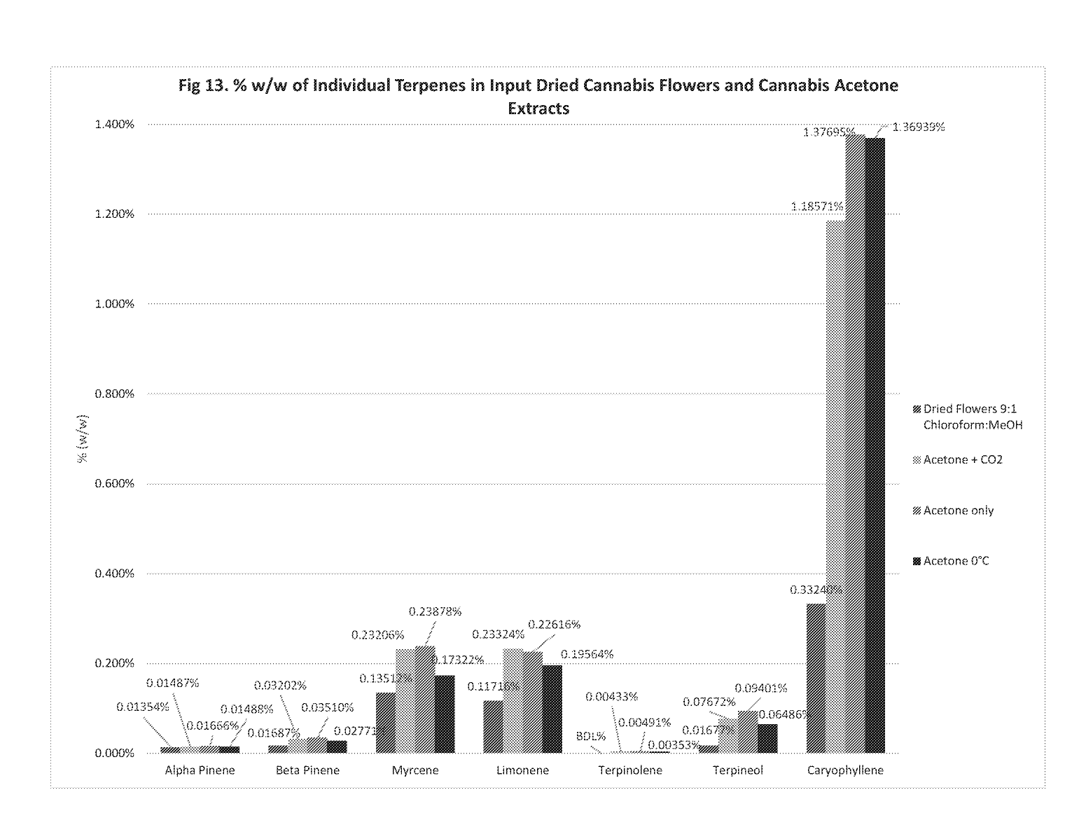

FIG. 13 shows % w/w of individual terpenes content in input dried cannabis flowers and cannabis extracts samples using acetone solvent systems disclosed herein. The data presented in FIG. 13 figure show the % w/w of individual terpenes compared between the dried cannabis flowers extracted with the validated 9:1 chloroform:Methanol method, and the extracted cannabis resins produced using acetone in the three example embodiment solvent systems tested (‘acetone+CO₂at about −78.5° C.’; ‘acetone onlyat about −78.5° C.’; ‘acetone only, 0° C.’). As can be seen, the overall trend observed when comparing all acetone extract sample sets with the results produced by 9:1 chloroform:methanol extraction (‘acetone+CO₂at about −78.5° C.’ vs. ‘acetone only −78.5° C.’ vs. ‘acetone only 0° C.’ vs. dried flower 9:1 chloroform:MeOH) indicates that concentration of all terpenes in acetone extracts relative to the 9:1 chloroform:methanol extract of dried flowers. Additionally, the % w/w of terpenes in each of the acetone extraction samples show minimal variation when compared to one another. FIG. 13 demonstrates that all three of the example embodiment acetone solvent systems tested provide a superior result compared with the 9:1 chloroform:methanol extraction procedure used for validation.

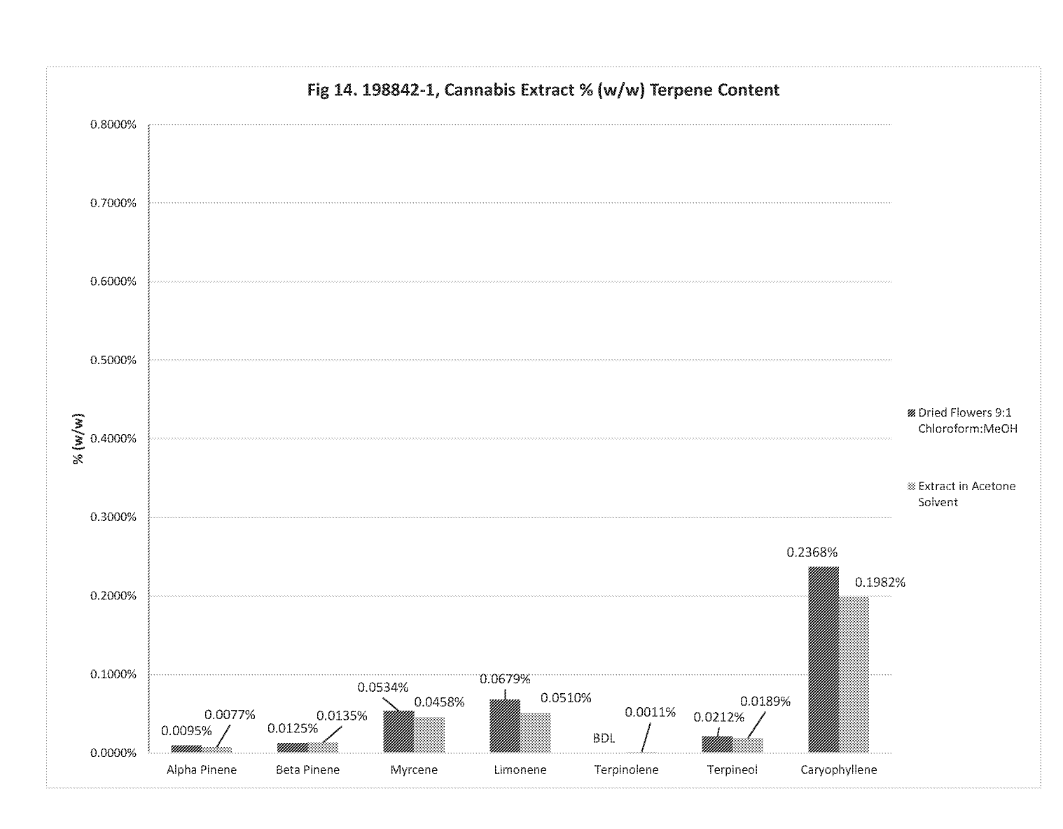

FIG. 14 shows % w/w terpene content in a cannabis extract sample designated as 198842-1. The extract sample in FIG. 14 was prepared with ‘acetone +CO₂ at about −78.5° C.’. The proportionality of terpenes identified in the dried cannabis flowers extract of sample 198842-1 appears to be maintained when extracted with acetone. Although the extract in acetone sample was dissolved in a volume of acetone that was 10-fold the mass of the dried cannabis flowers, the individual terpenes were detected at a concentration that was greater than the expected 10-fold dilution (ie. 0.0125% beta-pinene in dried flowers, would have been expected to yield a 0.00125% beta-pinene in the extract in acetone solvent). Thus, it will be appreciated that the example embodiment ‘acetone+CO2 at about −78.5° C.’ extraction system is superior at extracting terpenes relative to the ‘9:1 chloroform:methanol’ system.

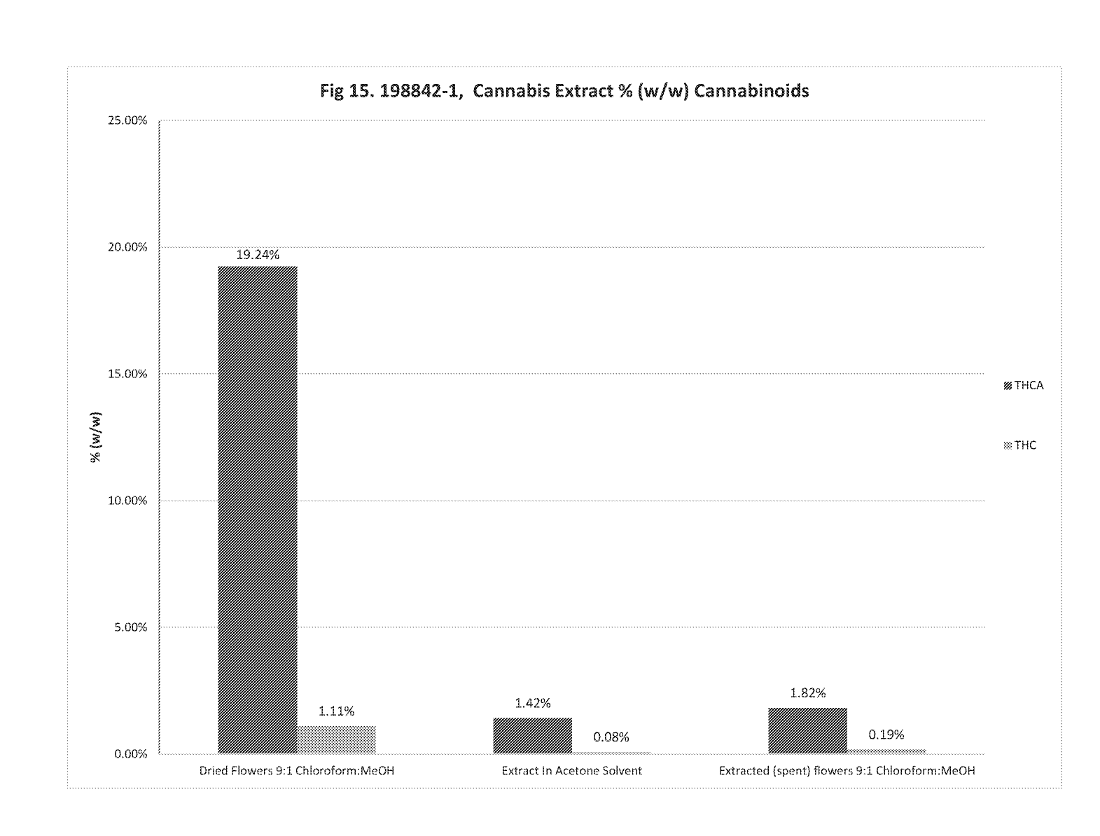

FIG. 15 show % w/w cannabinoids content in a cannabis extract sample designated as 198842-1. Comparing extracted (spent) flowers with the input dried flowers, it can be calculated that 90.54% THCA and 82.88% of THC was extracted from the input dried flowers with an acetone+CO₂at about −78.5° C.’ extraction. THCA and THC are found in approximately 10-fold diluted quantities as expected in the ‘extract in solvent’ sample and the ratio of THCA:THC (17.38) in the dried flowers is maintained in the Extract in solvent (17.75). This data shows a high yield of cannabinoid extraction with acetone, providing evidence that acetone works efficiently as a solvent for the extraction of non-polar cannabinoids while maintaining the natural ratios of the cannabinoids found in the cannabis flower.

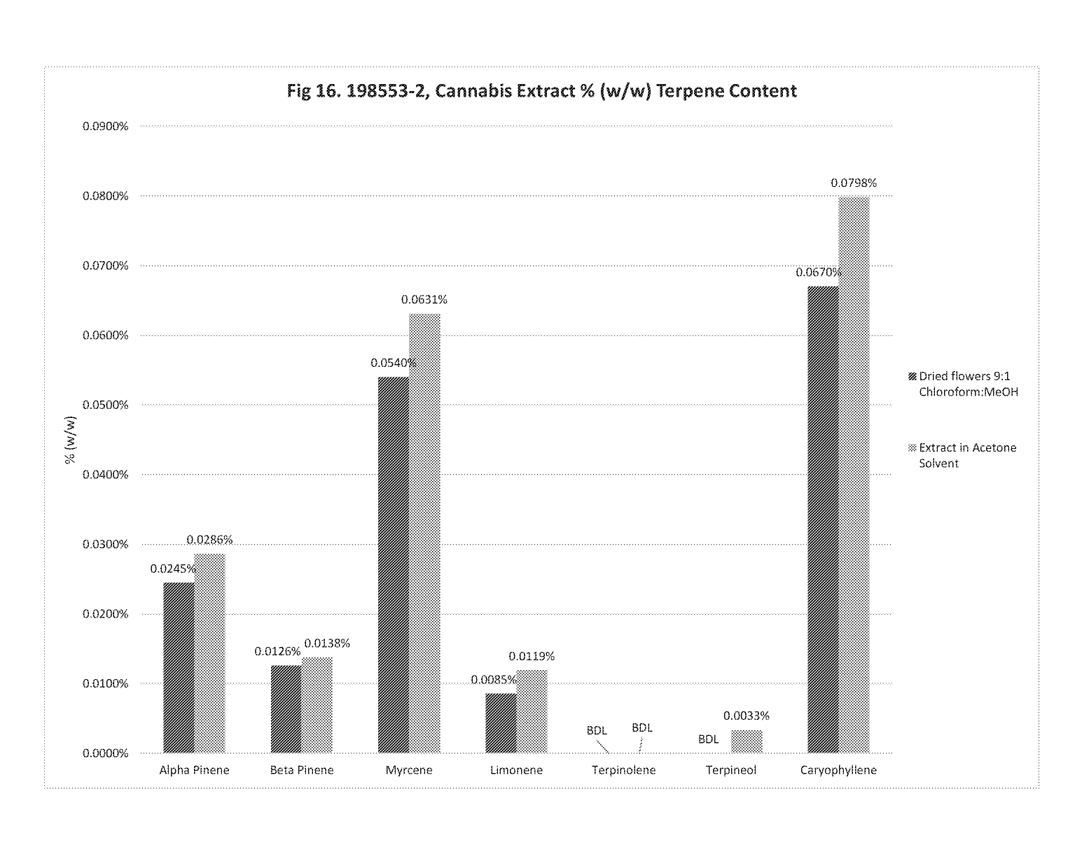

FIG. 16 shows % w/w terpene content in a cannabis extract sample designated as 198553-2. The extract sample in FIG. 16 was prepared with acetone +CO₂ at about −78.5° C.’. The proportionality of terpenes identified in the dried cannabis flowers extract of sample 198553-2 appears to be maintained when extracted with acetone. Although the cannabis extract has been diluted with a mass of acetone 10-fold that of the mass of input cannabis flowers that subjected to extraction, the % w/w of all terpenes (except for terpinolene) in acetone was greater relative to the ‘dried flowers’, indicating a high yield and high efficiency of terpene extraction. The result of 0.0033% terpineol in the extract in solvent further exemplifies the superior ability of the example embodiment extraction method(s) over the 9:1 chloroform:methanol method to efficiently concentrate terpenes such that the amount of terpineol present in the extraction solvent is above the detectable limit of the analytical device.

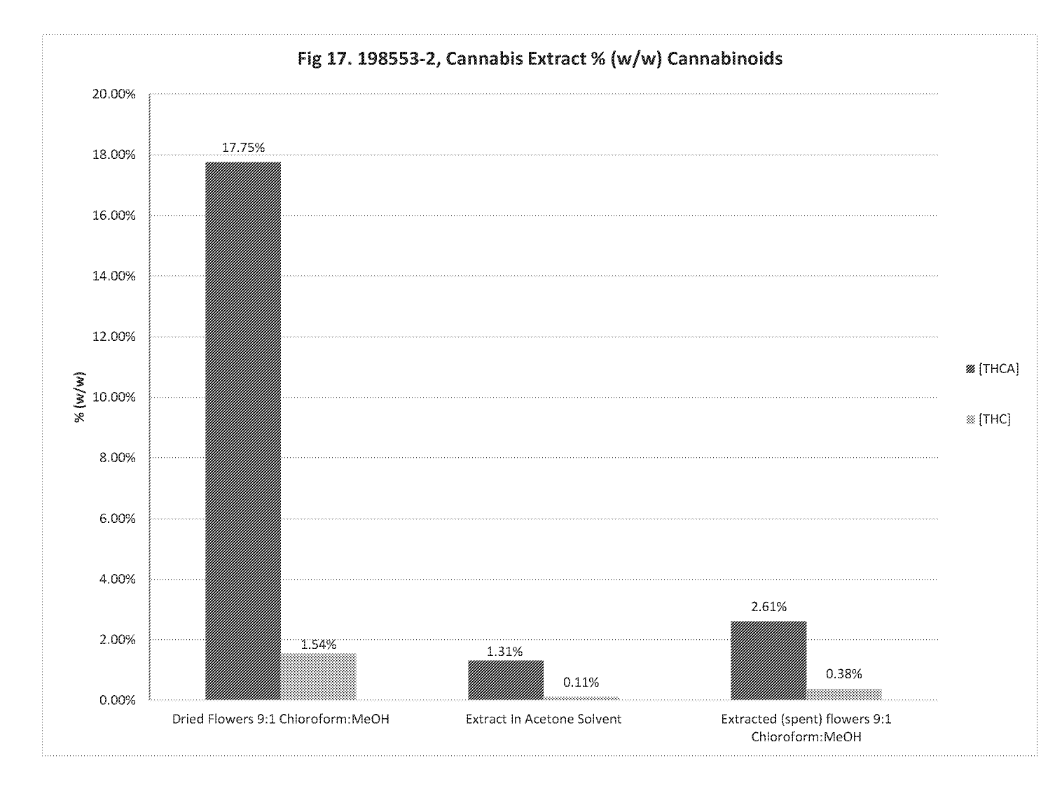

FIG. 17 shows % w/w cannabinoids content in a cannabis extract sample designated as 198553-2. It will be appreciated that 85.30% THCA and 75.32% of the available THC was extracted from the input dried flowers (calculated from a comparison of the cannabinoids in the input dried flowers and the remaining cannabinoids in the extracted flowers). Thus, these data indicate acetone's high capacity to extract non-polar cannabinoids. The THCA:THC ratio in the dried flower sample (11.53) was maintained without any significant change in the extract in acetone solvent sample (11.91).

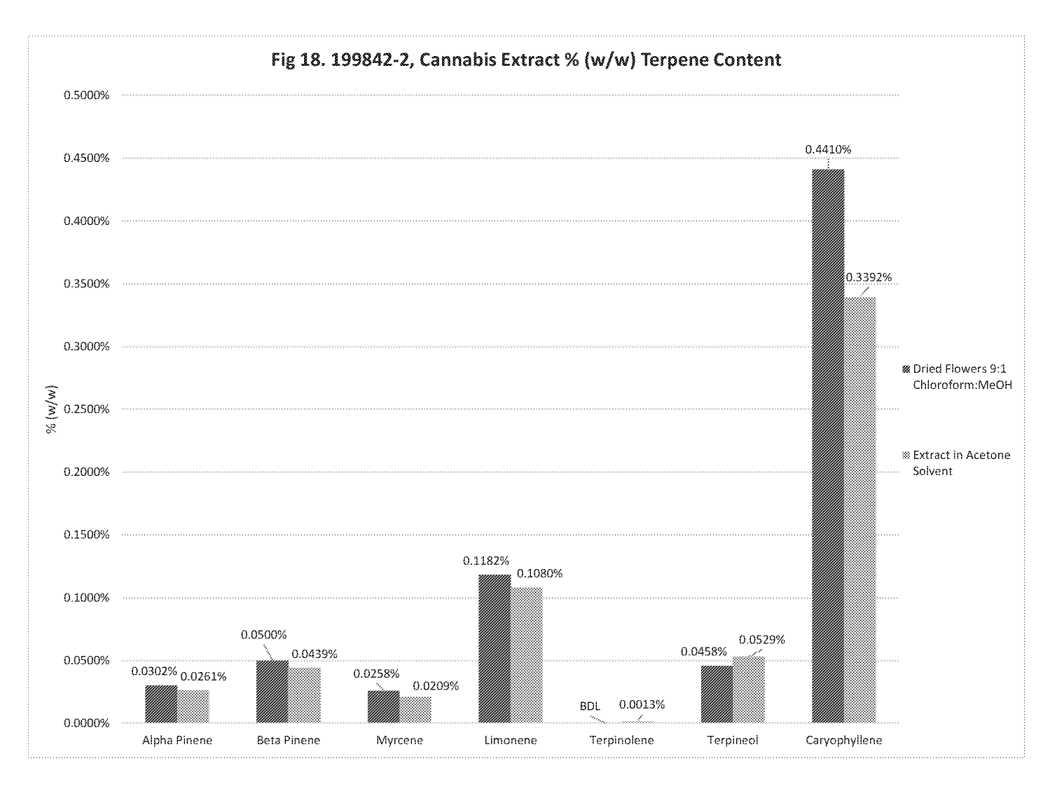

FIG. 18 shows % w/w terpene content in a cannabis extract sample designated as 198842-2. Extract sample in FIG. 18 was prepared using acetone +CO₂ at about −78.5° C.’. The proportionality of terpenes identified in the dried cannabis flowers extract of sample 198842-2 appears to be maintained when extracted with acetone. Although the cannabis extract has been diluted with a mass of acetone 10-fold that of the mass of input cannabis flowers that subjected to extraction, the % w/w of all terpenes in the solvent was greater than the expected amount from a 10-fold dilution, indicating a high yield and high efficiency of terpene extraction with the ‘acetone+CO₂ at about −78.5° C.’ system compared to the ‘9:1 chloroform:methanol extraction system’. Similar to terpineol results from FIG. 16, detectable amounts of terpinolene in the diluted extract in solvent sample exemplify the ability of the acetone extraction to extract and concentrate terpenes more efficiently than the validated extraction protocol using 9:1 chloroform:methanol that was used to prepared all samples identified as ‘dried flowers’.

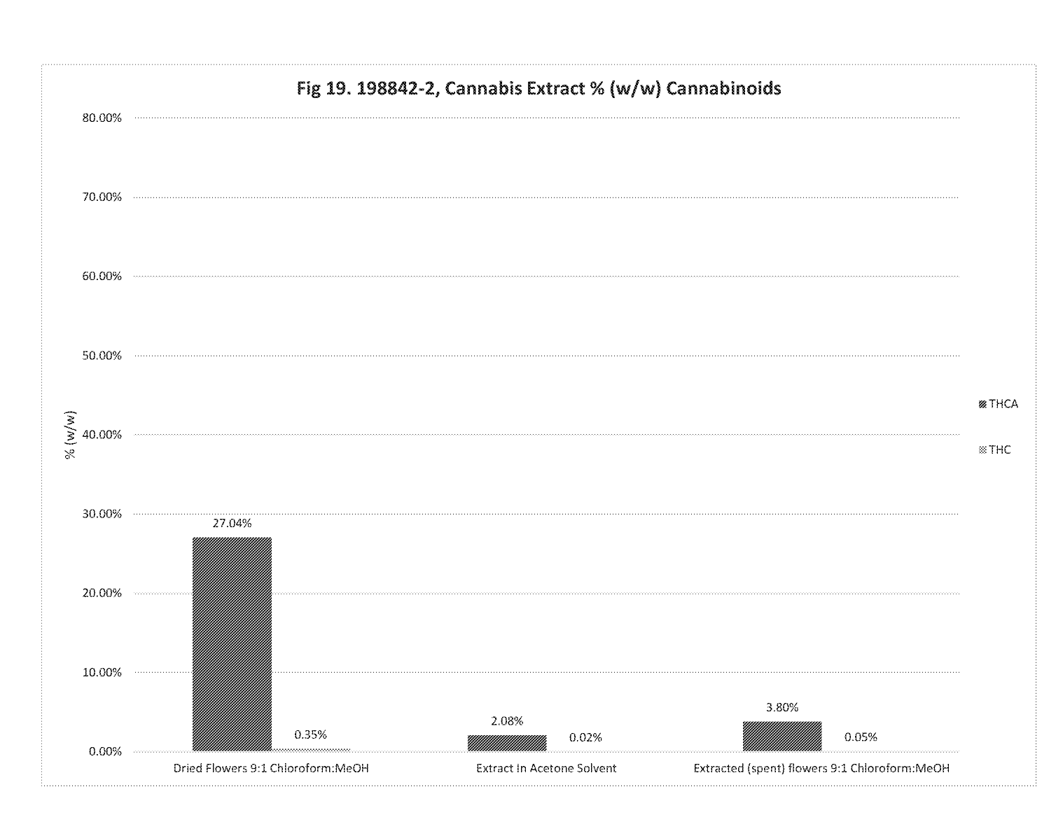

FIG. 19 shows % w/w cannabinoids content in a cannabis extract sample designated as 198842-2. As can be seen, 85.95% THCA and 86.11% THC extracted from dried flowers (comparing input dried flowers vs extracted flowers), indicating acetone's high capacity to extract non-polar cannabinoids.

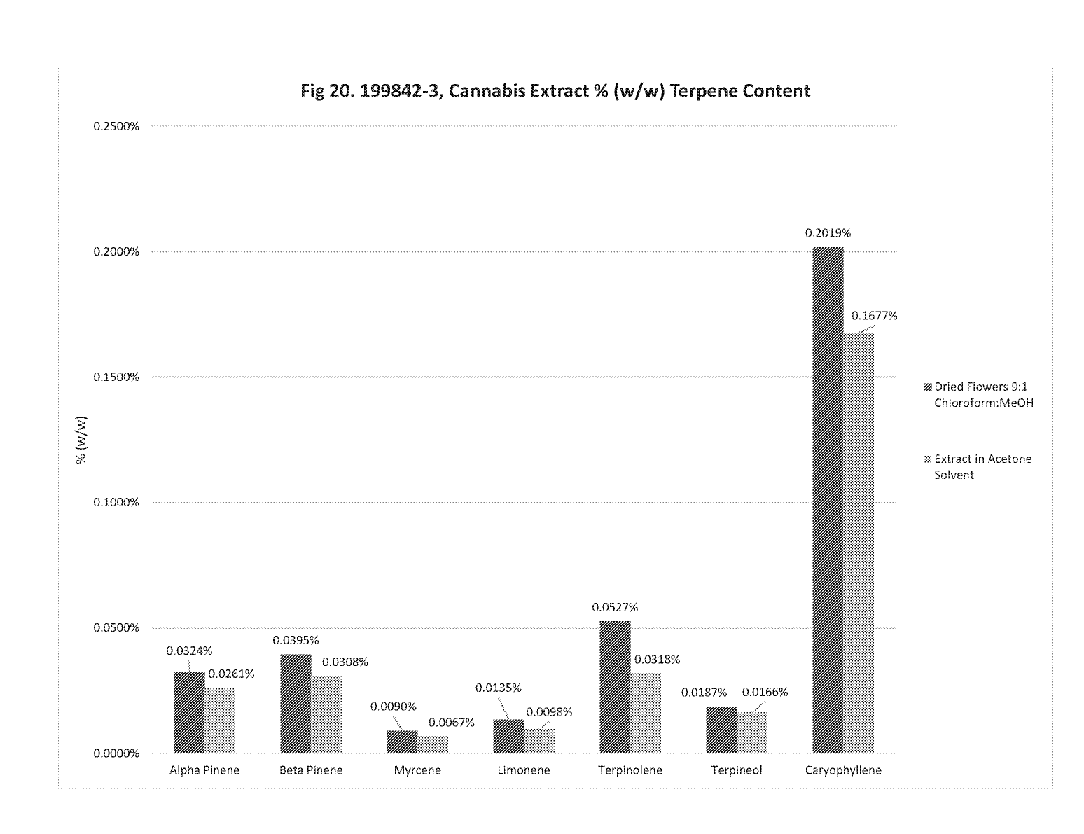

FIG. 20 shows % w/w terpene content in a cannabis extract sample designated as 198842-3, which was prepared using acetone+CO₂ at about −78.5° C.’. The proportionality of terpenes identified in the dried cannabis flowers extract of sample 198842-3 was substantially be maintained when extracted with acetone. Although the cannabis extract has been diluted with a mass of acetone 10-fold that of the mass of input cannabis flowers that subjected to extraction, the % w/w of all terpenes in the solvent was greater than the expected amount from a 10-fold dilution, indicating a high yield and high efficiency of terpene extraction.

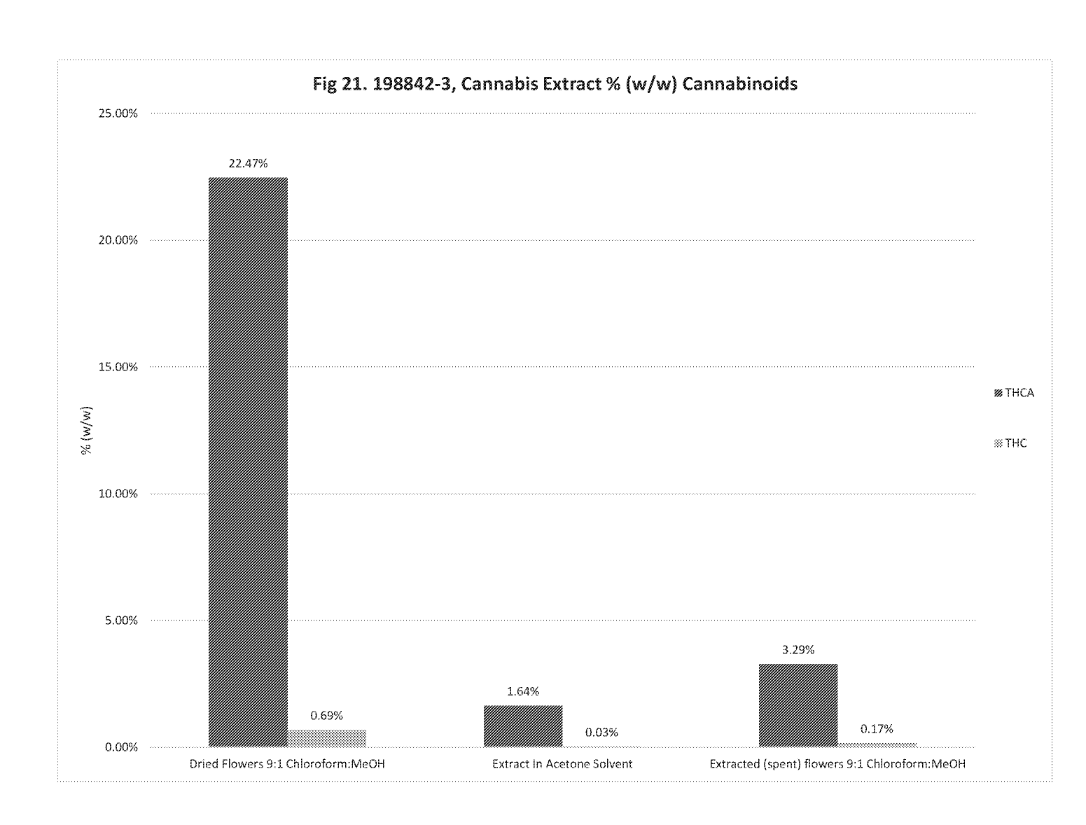

FIG. 21 shows % w/w cannabinoid content in a cannabis extract sample designated as 198842-3. As can be seen, 85.36% THCA and 75.36% THC was extracted from dried flowers (comparing input dried flowers vs extracted flowers), indicating acetone's high capacity to extract non-polar cannabinoids.

To the extent necessary to provide descriptive support, it shall be understood that the subject matter and/or text of any appended claims are incorporated herein by reference in their entirety.

It will be understood by all readers of this written description that the example embodiments described herein may be suitably practiced in the absence of any recited feature, element or step that is, or is not, specifically disclosed herein.

1. A method for producing an extract from a botanical material, wherein the extract contains at least one target compound, the method comprising:
admixing the botanical material with acetone and dry ice to obtain a mixture;
allowing the temperature of the mixture to reach about −78.5° C.;
optionally agitating the mixture;
filtering the mixture to remove to obtain a filtrate; and
removing the solvent from the filtrate to obtain the extract.

2. The method of claim 1, wherein the botanical material is a member of the Angiosperm family selected from the group consisting of: Acanthaceae; Achariaceae; Achatocarpaceae; Acoraceae; Actinidiaceae; Adoxaceae; Aextoxicaceae; Aizoaceae; Akaniaceae; Alismataceae; Alseuosmiaceae; Alstroemeriaceae; Altingiaceae; Amaranthaceae; Amaryllidaceae; Amborellaceae; Anacampserotaceae; Anacardiaceae; Anarthriaceae; Ancistrocladaceae; Anisophylleaceae; Annonaceae; Aphanopetalaceae; Aphloiaceae; Apiaceae; Apocynaceae; Apodanthaceae; Aponogetonaceae; Aquifoliaceae; Araceae; Araliaceae; Arecaceae; Argophyllaceae; Aristolochiaceae; Asparagaceae; Asteliaceae; Asteropeiaceae; Atherospermataceae; Austrobaileyaceae; Balanopaceae; Balanophoraceae; Balsaminaceae; Barbeuiaceae; Barbeyaceae; Basellaceae; Bataceae; Begoniaceae; Berberidaceae; Berberidopsidaceae; Betulaceae; Biebersteiniaceae; Bignoniaceae; Bixaceae; Blandfordiaceae; Bonnetiaceae; Boraginaceae; Boryaceae; Brassicaceae; Bromeliaceae; Brunelliaceae; Bruniaceae; Burmanniaceae; Burseraceae; Butomaceae; Buxaceae; Byblidaceae; Cabombaceae; Cactaceae; Calceolariaceae; Calophyllaceae; Calycanthaceae; Calyceraceae; Campanulaceae; Campynemataceae; Canellaceae; Cannabaceae; Cannaceae; Capparaceae; Caprifoliaceae; Cardiopteridaceae; Caricaceae; Carlemanniaceae; Caryocaraceae; Caryophyllaceae; Casuarinaceae; Celastraceae; Centrolepidaceae; Centroplacaceae; Cephalotaceae; Ceratophyllaceae; Cercidiphyllaceae; Chloranthaceae; Chrysobalanaceae; Circaeasteraceae; Cistaceae; Cleomaceae; Clethraceae; Clusiaceae; Colchicaceae; Columelliaceae; Combretaceae; Commelinaceae; Compositae; Connaraceae; Convolvulaceae; Coriariaceae; Cornaceae; Corsiaceae; Corynocarpaceae; Costaceae; Crassulaceae; Crossosomataceae; Ctenolophonaceae; Cucurbitaceae; Cunoniaceae; Curtisiaceae; Cyclanthaceae; Cymodoceaceae; Cynomoriaceae; Cyperaceae; Cyrillaceae; Cytinaceae; Daphniphyllaceae; Dasypogonaceae; Datiscaceae; Degeneriaceae; Diapensiaceae; Dichapetalaceae; Didiereaceae; Dilleniaceae; Dioncophyllaceae; Dioscoreaceae; Dipentodontaceae; Dipterocarpaceae; Dirachmaceae; Doryanthaceae; Droseraceae; Drosophyllaceae; Ebenaceae; Ecdeiocoleaceae; Elaeagnaceae; Elaeocarpaceae; Elatinaceae; Emblingiaceae; Ericaceae; Eriocaulaceae; Erythroxylaceae; Escalloniaceae; Eucommiaceae; Euphorbiaceae; Euphroniaceae; Eupomatiaceae; Eupteleaceae; Fagaceae; Flacourtiaceae; Flagellariaceae; Fouquieriaceae; Frankeniaceae; Garryaceae; Geissolomataceae; Gelsemiaceae; Gentianaceae; Geraniaceae; Gerrardinaceae; Gesneriaceae; Gisekiaceae; Gomortegaceae; Goodeniaceae; Goupiaceae; Grossulariaceae; Grubbiaceae; Guamatelaceae; Gunneraceae; Gyrostemonaceae; Haemodoraceae; Halophytaceae; Haloragaceae; Hamamelidaceae; Hanguanaceae; Haptanthaceae; Heliconiaceae; Helwingiaceae; Hernandiaceae; Himantandraceae; Huaceae; Humiriaceae; Hydatellaceae; Hydnoraceae; Hydrangeaceae; Hydrocharitaceae; Hydroleaceae; Hydrostachyaceae; Hypericaceae; Hypoxidaceae; Icacinaceae; Iridaceae; Irvingiaceae; Iteaceae; Ixioliriaceae; Ixonanthaceae; Joinvilleaceae; Juglandaceae; Juncaceae; Juncaginaceae; Kirkiaceae; Koeberliniaceae; Krameriaceae; Lacistemataceae; Lactoridaceae; Lamiaceae; Lanariaceae; Lardizabalaceae; Lauraceae; Lecythidaceae; Leguminosae; Lentibulariaceae; Lepidobotryaceae; Liliaceae; Limeaceae; Limnanthaceae; Linaceae; Linderniaceae; Loasaceae; Loganiaceae; Lophiocarpaceae; Lophopyxidaceae; Loranthaceae; Lowiaceae; Lythraceae; Magnoliaceae; Malpighiaceae; Malvaceae; Marantaceae; Marcgraviaceae; Martyniaceae; Mayacaceae; Melanthiaceae; Melastomataceae; Meliaceae; Melianthaceae; Menispermaceae; Menyanthaceae; Metteniusaceae; Misodendraceae; Mitrastemonaceae; Molluginaceae; Monimiaceae; Montiaceae; Montiniaceae; Moraceae; Moringaceae; Muntingiaceae; Musaceae; Myodocarpaceae; Myricaceae; Myristicaceae; Myrothamnaceae; Myrtaceae; Nartheciaceae; Nelumbonaceae; Nepenthaceae; Neuradaceae; Nitrariaceae; Nothofagaceae; Nyctaginaceae; Nymphaeaceae; Ochnaceae; Olacaceae; Oleaceae; Onagraceae; Oncothecaceae; Opiliaceae; Orchidaceae; Orobanchaceae; Oxalidaceae; Paeoniaceae; Pandaceae; Pandanaceae; Papaveraceae; Paracryphiaceae; Passifloraceae; Paulowniaceae; Pedaliaceae; Penaeaceae; Pennantiaceae; Pentadiplandraceae; Pentaphragmataceae; Pentaphylacaceae; Penthoraceae; Peraceae; Peridiscaceae; Petenaeaceae; Petermanniaceae; Petrosaviaceae; Phellinaceae; Philesiaceae; Philydraceae; Phrymaceae; Phyllanthaceae; Phyllonomaceae; Physenaceae; Phytolaccaceae; Picramniaceae; Picrodendraceae; Piperaceae; Pittosporaceae; Plantaginaceae; Platanaceae; Plocospermataceae; Plumbaginaceae; Poaceae; Podostemaceae; Polemoniaceae; Polygalaceae; Polygonaceae; Pontederiaceae; Portulacaceae; Posidoniaceae; Potamogetonaceae; Primulaceae; Proteaceae; Putranjivaceae; Quillajaceae; Rafflesiaceae; Ranunculaceae; Rapateaceae; Resedaceae; Restionaceae; Rhabdodendraceae; Rhamnaceae; Rhipogonaceae; Rhizophoraceae; Roridulaceae; Rosaceae; Rousseaceae; Rubiaceae; Ruppiaceae; Rutaceae; Sabiaceae; Salicaceae; Salvadoraceae; Santalaceae; Sapindaceae; Sapotaceae; Sarcobataceae; Sarcolaenaceae; Sarraceniaceae; Saururaceae; Saxifragaceae; Scheuchzeriaceae; Schisandraceae; Schlegeliaceae; Schoepfiaceae; Scrophulariaceae; Setchellanthaceae; Simaroubaceae; Simmondsiaceae; Siparunaceae; Sladeniaceae; Smilacaceae; Solanaceae; Sphaerosepalaceae; Sphenocleaceae; Stachyuraceae; Staphyleaceae; Stegnospermataceae; Stemonaceae; Stemonuraceae; Stilbaceae; Strasburgeriaceae; Strelitziaceae; Stylidiaceae; Styracaceae; Surianaceae; Symplocaceae; Talinaceae; Tamaricaceae; Tapisciaceae; Tecophilaeaceae; Tetrachondraceae; Tetramelaceae; Tetrameristaceae; Theaceae; Thomandersiaceae; Thurniaceae; Thymelaeaceae; Ticodendraceae; Tofieldiaceae; Torricelliaceae; Tovariaceae; Trigoniaceae; Trimeniaceae; Triuridaceae; Trochodendraceae; Tropaeolaceae; Typhaceae; Ulmaceae; Urticaceae; Vahliaceae; Velloziaceae; Verbenaceae; Violaceae; Vitaceae; Vivianiaceae; Vochysiaceae; Winteraceae; Xanthorrhoeaceae; Xeronemataceae; Xyridaceae; Zingiberaceae; Zosteraceae; and Zygophyllaceae.

3. The method of claim 2, wherein the botanical material is cannabis.

4. The method of claim 2, wherein the botanical material is tobacco.

5. The method of claim 1, wherein the botanical material, acetone and dry ice are admixed in a vessel comprising stainless steel or glass.

6. The method of claim 5, wherein the amount (w/w) of acetone to botanical material that is present in the vessel is about 15:1, 10:1, 5:1, 4:1, 3:1, 2:1, 1:1, 1:2, 1:3, 1:4, 1:5, 1:10, or 1:15.

7. The method of claim 5, wherein the amount (w/w) of dry ice to botanical material present in the vessel is about 15:1, 10:1, 5:1, 4:1, 3:1, 2:1, 1:1, 1:2, 1:3, 1:4, 1:5, 1:10, or 1:15.

8. The method of claim 1, wherein the agitating step is performed for 1 to 360 minutes.

9. The method of claim 1, wherein the agitating step is performed for 10 to 60 minutes.

10. The method of claim 1, wherein the dry ice and acetone are added to the vessel prior to adding the botanical material.

11. The method of claim 1, wherein the dry ice is added to the vessel prior to the botanical material and acetone.

12. The method of claim 1
wherein the temperature of the mixture is allowed to to reach −78.5° C. to 0° C.

13. The method of claim 12, wherein the botanical material is a member of the Angiosperm family selected from the group consisting of: Acanthaceae; Achariaceae; Achatocarpaceae; Acoraceae; Actinidiaceae; Adoxaceae; Aextoxicaceae; Aizoaceae; Akaniaceae; Alismataceae; Alseuosmiaceae; Alstroemeriaceae; Altingiaceae; Amaranthaceae; Amaryllidaceae; Amborellaceae; Anacampserotaceae; Anacardiaceae; Anarthriaceae; Ancistrocladaceae; Anisophylleaceae; Annonaceae; Aphanopetalaceae; Aphloiaceae; Apiaceae; Apocynaceae; Apodanthaceae; Aponogetonaceae; Aquifoliaceae; Araceae; Araliaceae; Arecaceae; Argophyllaceae; Aristolochiaceae; Asparagaceae; Asteliaceae; Asteropeiaceae; Atherospermataceae; Austrobaileyaceae; Balanopaceae; Balanophoraceae; Balsaminaceae; Barbeuiaceae; Barbeyaceae; Basellaceae; Bataceae; Begoniaceae; Berberidaceae; Berberidopsidaceae; Betulaceae; Biebersteiniaceae; Bignoniaceae; Bixaceae; Blandfordiaceae; Bonnetiaceae; Boraginaceae; Boryaceae; Brassicaceae; Bromeliaceae; Brunelliaceae; Bruniaceae; Burmanniaceae; Burseraceae; Butomaceae; Buxaceae; Byblidaceae; Cabombaceae; Cactaceae; Calceolariaceae; Calophyllaceae; Calycanthaceae; Calyceraceae; Campanulaceae; Campynemataceae; Canellaceae; Cannabaceae; Cannaceae; Capparaceae; Caprifoliaceae; Cardiopteridaceae; Caricaceae; Carlemanniaceae; Caryocaraceae; Caryophyllaceae; Casuarinaceae; Celastraceae; Centrolepidaceae; Centroplacaceae; Cephalotaceae; Ceratophyllaceae; Cercidiphyllaceae; Chloranthaceae; Chrysobalanaceae; Circaeasteraceae; Cistaceae; Cleomaceae; Clethraceae; Clusiaceae; Colchicaceae; Columelliaceae; Combretaceae; Commelinaceae; Compositae; Connaraceae; Convolvulaceae; Coriariaceae; Cornaceae; Corsiaceae; Corynocarpaceae; Costaceae; Crassulaceae; Crossosomataceae; Ctenolophonaceae; Cucurbitaceae; Cunoniaceae; Curtisiaceae; Cyclanthaceae; Cymodoceaceae; Cynomoriaceae; Cyperaceae; Cyrillaceae; Cytinaceae; Daphniphyllaceae; Dasypogonaceae; Datiscaceae; Degeneriaceae; Diapensiaceae; Dichapetalaceae; Didiereaceae; Dilleniaceae; Dioncophyllaceae; Dioscoreaceae; Dipentodontaceae; Dipterocarpaceae; Dirachmaceae; Doryanthaceae; Droseraceae; Drosophyllaceae; Ebenaceae; Ecdeiocoleaceae; Elaeagnaceae; Elaeocarpaceae; Elatinaceae; Emblingiaceae; Ericaceae; Eriocaulaceae; Erythroxylaceae; Escalloniaceae; Eucommiaceae; Euphorbiaceae; Euphroniaceae; Eupomatiaceae; Eupteleaceae; Fagaceae; Flacourtiaceae; Flagellariaceae; Fouquieriaceae; Frankeniaceae; Garryaceae; Geissolomataceae; Gelsemiaceae; Gentianaceae; Geraniaceae; Gerrardinaceae; Gesneriaceae; Gisekiaceae; Gomortegaceae; Goodeniaceae; Goupiaceae; Grossulariaceae; Grubbiaceae; Guamatelaceae; Gunneraceae; Gyrostemonaceae; Haemodoraceae; Halophytaceae; Haloragaceae; Hamamelidaceae; Hanguanaceae; Haptanthaceae; Heliconiaceae; Helwingiaceae; Hernandiaceae; Himantandraceae; Huaceae; Humiriaceae; Hydatellaceae; Hydnoraceae; Hydrangeaceae; Hydrocharitaceae; Hydroleaceae; Hydrostachyaceae; Hypericaceae; Hypoxidaceae; Icacinaceae; Iridaceae; Irvingiaceae; Iteaceae; Ixioliriaceae; Ixonanthaceae; Joinvilleaceae; Juglandaceae; Juncaceae; Juncaginaceae; Kirkiaceae; Koeberliniaceae; Krameriaceae; Lacistemataceae; Lactoridaceae; Lamiaceae; Lanariaceae; Lardizabalaceae; Lauraceae; Lecythidaceae; Leguminosae; Lentibulariaceae; Lepidobotryaceae; Liliaceae; Limeaceae; Limnanthaceae; Linaceae; Linderniaceae; Loasaceae; Loganiaceae; Lophiocarpaceae; Lophopyxidaceae; Loranthaceae; Lowiaceae; Lythraceae; Magnoliaceae; Malpighiaceae; Malvaceae; Marantaceae; Marcgraviaceae; Martyniaceae; Mayacaceae; Melanthiaceae; Melastomataceae; Meliaceae; Melianthaceae; Menispermaceae; Menyanthaceae; Metteniusaceae; Misodendraceae; Mitrastemonaceae; Molluginaceae; Monimiaceae; Montiaceae; Montiniaceae; Moraceae; Moringaceae; Muntingiaceae; Musaceae; Myodocarpaceae; Myricaceae; Myristicaceae; Myrothamnaceae; Myrtaceae; Nartheciaceae; Nelumbonaceae; Nepenthaceae; Neuradaceae; Nitrariaceae; Nothofagaceae; Nyctaginaceae; Nymphaeaceae; Ochnaceae; Olacaceae; Oleaceae; Onagraceae; Oncothecaceae; Opiliaceae; Orchidaceae; Orobanchaceae; Oxalidaceae; Paeoniaceae; Pandaceae; Pandanaceae; Papaveraceae; Paracryphiaceae; Passifloraceae; Paulowniaceae; Pedaliaceae; Penaeaceae; Pennantiaceae; Pentadiplandraceae; Pentaphragmataceae; Pentaphylacaceae; Penthoraceae; Peraceae; Peridiscaceae; Petenaeaceae; Petermanniaceae; Petrosaviaceae; Phellinaceae; Philesiaceae; Philydraceae; Phrymaceae; Phyllanthaceae; Phyllonomaceae; Physenaceae; Phytolaccaceae; Picramniaceae; Picrodendraceae; Piperaceae; Pittosporaceae; Plantaginaceae; Platanaceae; Plocospermataceae; Plumbaginaceae; Poaceae; Podostemaceae; Polemoniaceae; Polygalaceae; Polygonaceae; Pontederiaceae; Portulacaceae; Posidoniaceae; Potamogetonaceae; Primulaceae; Proteaceae; Putranjivaceae; Quillajaceae; Rafflesiaceae; Ranunculaceae; Rapateaceae; Resedaceae; Restionaceae; Rhabdodendraceae; Rhamnaceae; Rhipogonaceae; Rhizophoraceae; Roridulaceae; Rosaceae; Rousseaceae; Rubiaceae; Ruppiaceae; Rutaceae; Sabiaceae; Salicaceae; Salvadoraceae; Santalaceae; Sapindaceae; Sapotaceae; Sarcobataceae; Sarcolaenaceae; Sarraceniaceae; Saururaceae; Saxifragaceae; Scheuchzeriaceae; Schisandraceae; Schlegeliaceae; Schoepfiaceae; Scrophulariaceae; Setchellanthaceae; Simaroubaceae; Simmondsiaceae; Siparunaceae; Sladeniaceae; Smilacaceae; Solanaceae; Sphaerosepalaceae; Sphenocleaceae; Stachyuraceae; Staphyleaceae; Stegnospermataceae; Stemonaceae; Stemonuraceae; Stilbaceae; Strasburgeriaceae; Strelitziaceae; Stylidiaceae; Styracaceae; Surianaceae; Symplocaceae; Talinaceae; Tamaricaceae; Tapisciaceae; Tecophilaeaceae; Tetrachondraceae; Tetramelaceae; Tetrameristaceae; Theaceae; Thomandersiaceae; Thurniaceae; Thymelaeaceae; Ticodendraceae; Tofieldiaceae; Torricelliaceae; Tovariaceae; Trigoniaceae; Trimeniaceae; Triuridaceae; Trochodendraceae; Tropaeolaceae; Typhaceae; Ulmaceae; Urticaceae; Vahliaceae; Velloziaceae; Verbenaceae; Violaceae; Vitaceae; Vivianiaceae; Vochysiaceae; Winteraceae; Xanthorrhoeaceae; Xeronemataceae; Xyridaceae; Zingiberaceae; Zosteraceae; and Zygophyllaceae.

14. The method of claim 13, wherein the botanical material is cannabis.

15. The method of claim 13, wherein the botanical material is tobacco.

16. The method of claim 12, wherein the botanical material, acetone and dry ice are admixed in a vessel comprising stainless steel or glass.

17. The method of claim 16, wherein the amount (w/w) of acetone to botanical material that is present in the vessel is about 15:1, 10:1, 5:1, 4:1, 3:1, 2:1, 1:1, 1:2, 1:3, 1:4, 1:5, 1:10, or 1:15.

18. The method of claim 16, wherein the amount (w/w) of dry ice to botanical material present in the vessel is about 15:1, 10:1, 5:1, 4:1, 3:1, 2:1, 1:1, 1:2, 1:3, 1:4, 1:5, 1:10, or 1:15.

19. The method of claim 12, wherein the agitating step is performed for 1 to 360 minutes.

20. The method of claim 12, wherein the agitating step is performed for 10 to 360 minutes.

21. The method of claim 12, wherein the acetone is added to the vessel prior to adding the botanical material.

22. The method of claim 12, wherein the botanical material is added to the vessel prior to the acetone.

23. The method of claim 1, wherein the extract is a liquid or semi-solid.

24. The method of claim 1, wherein the extract comprises at least one compound selected from a member of the group consisting of terpenes, terpenoids, cannabinoids, flavonoids and alkaloids.

25. The method of claim 1, wherein the extract comprises at least one compound selected from a member of the group consisting of CBD, THC and nicotine.

26. The method of claim 1, wherein the extract comprises at least two compounds selected from a member of the group consisting of terpenes, terpenoids, cannabinoids, flavonoids and alkaloids.

27. The method of claim 1, wherein the extract is substantially free of chlorophyll, pigments, waxes, fats and lipids.

28. An extract obtained by the method of claim 1.

29. A container comprising the extract of claim 28.

30. A therapeutic composition comprising an extract of claim 28 and a therapeutically acceptable or inert carrier.
